(19) United States
(12) Patent Application Publication
(10) Pub. No.: US 2021/0346385 A1
(43) Pub. Date: Nov. 11, 2021

(54) TASTE-MASKED DOSAGE FORMS

(71) Applicant: MSB Holdings, Inc, Austin, TX (US)

(72) Inventor: Kenwaljit Singh Bains, Austin, TX (US)

(21) Appl. No.: 17/277,939

(22) PCT Filed: Sep. 23, 2019
(86) PCT No.: PCT/US2019/052504

§ 371 Date: Mar. 19, 2021

Related U.S. Application Data
(60) Provisional application No. 62/882,325, filed on Aug. 2, 2019.
(60) Provisional application No. 62/755,769, filed on Nov. 5, 2018.
(60) Provisional application No. 62/734,810, filed on Sep. 21, 2018.

(51) Int. Cl. A61K 31/519; A61K 31/4985; A61K 38/095; A61K 31/568; A61K 9/00; A61K 47/36; A61K 47/22; A61K 47/44; A61K 47/46; A61K 47/26; A61K 36/185; A61K 31/05; A61K 31/352

(52) U.S. Cl. CPC A61K 31/519; A61K 31/4985; A61K 38/095; A61K 31/568; A61K 9/0056; A61K 47/36; A61K 31/352; A61K 47/44; A61K 47/46; A61K 47/26; A61K 36/185; A61K 31/05; A61K 47/22

(57) ABSTRACT

Sublingual dosage forms comprising a bitter active ingredient (including sildenafil, tadalafil, tetrahydrocannabinol, and cannabidiol) and a bitterness reducing agent are described. Methods of treating sexual dysfunction by administering the sublingual dosage form to an individual in need thereof are also described.

### CROSS REFERENCE TO RELATED APPLICATIONS

The present application claims the benefit of priority to U.S. Provisional Application Ser. No. 62/734,810, filed Sep. 21, 2018; U.S. Provisional Application Ser. No. 62/755,769, filed Nov. 5, 2018; and U.S. Provisional Application Ser. No. 62/882,325, filed Aug. 2, 2019, the contents of which are hereby incorporated by reference in their entirety.

### TECHNICAL FIELD

The present disclosure relates to taste-masked formulations comprising bitter active ingredients, including, in some embodiments, taste-masked formulations of cannabidiol, tetrahydrocannabinol, sildenafil, combinations of sildenafil and tadalafil, and opioids, as well as other bitter active agents disclosed herein.

### BACKGROUND OF THE DISCLOSURE

Sublingually- or buccally-administered dosage forms have many advantages. For example, sublingually- or buccally-administered active ingredients are rapidly absorbed (i.e., rapidly bioavailable), which provides rapid drug action. Rapid drug action is important in a number of indications, such as the treatment of erectile dysfunction where the patient requires a therapeutic effect shortly after administration. Sublingually- or buccally-administered drugs avoid first-pass metabolism, which often means that a sublingually-administered drug will be effective at lower dose than an orally-administered dose of the same drug (such as an oral tablet). For example, the cannabis extract constituents CBD and THC undergo extension first-pass metabolism, which limits their oral bioavailability and requires the administration of high oral doses to achieve therapeutic effects.

Furthermore, sublingually- or buccally-administered dosages do not require swallowing and are thus advantageous for administering to populations who have trouble swallowing pills or tablets (such as pediatric patients, the elderly, and patients with dysphagia). Unfortunately, many active ingredients have an extreme bitter taste when administered sublingually or buccally (bitter active ingredients) such that patients will not tolerate sublingual or buccal administration of the bitter active ingredient. The poor palatability can result in poor patient compliance with sublingual formulations containing bitter active ingredients.

Taste-masking bitter active ingredients is complex and, in addition to masking the bitter taste of the active ingredient, the formulation must be overall palatable (for example, not immediately bitter, no bitter aftertaste, not overly sweet, good mouthfeel) to provide a useful sublingual formulation of a bitter active ingredient. For example, masking bitterness by including a sweetener is often not sufficient to improve overall palatability.

There is a need in the art for formulations that effectively mask the bitter taste of active ingredients to provide palatable sublingual or buccal dosage forms containing bitter active ingredients.

The present disclosure provides, in some embodiments, taste-masked formulations comprising bitter active ingredients (including cannabis extracts, cannabidiol, tetrahydrocannabinol, sildenafil and combinations of sildenafil and tadalafil) and a bitterness reducing agent. The applicants discovered that the specific selection of bitterness reducing agents was much more effective than other, similar bitterness reducing agents.

### SUMMARY OF THE DISCLOSURE

In one aspect, the present disclosure provides compositions that mask the bitterness of a bitter active ingredient. In some embodiments, the composition comprises a bitterness reducing agent and a bitter active ingredient. In some embodiments, the composition is a sublingual dosage form. In some embodiments, the composition is in the form of a troche. In some embodiments, the composition is a buccal dosage form.

In some embodiments, the bitter active ingredient is sildenafil or a pharmaceutically acceptable salt, prodrug, or solvate thereof. In some embodiments, the bitter active ingredient is tadalafil or a pharmaceutically acceptable salt, prodrug, or solvate thereof. In some embodiments, the bitter active ingredient is a mixture of sildenafil and tadalafil.

In some embodiments, the bitter active ingredient is a cannabis extract. In some embodiments, the bitter active ingredient is cannabidiol or a pharmaceutically acceptable salt, ester, prodrug, or solvate thereof (“CBD”). In some embodiments, the bitter active ingredient is tetrahydrocannabinol or a pharmaceutically acceptable salt, ester, prodrug, or solvate thereof (“THC”). In some embodiments, the bitter active ingredient is a mixture of CBD and THC.

In one aspect, the present disclosure provides methods of treating sexual dysfunction in a patient in need thereof comprising administering a therapeutically effective dose of a composition comprising a bitterness reducing agent and sildenafil or a pharmaceutically acceptable salt, prodrug, or solvate thereof. The present disclosure provides methods of treating sexual dysfunction in a patient in need thereof comprising administering a therapeutically effective dose of a composition comprising a bitterness reducing agent and tadalafil or a pharmaceutically acceptable salt, prodrug, or solvate thereof. The present disclosure provides methods of treating sexual dysfunction in a patient in need thereof comprising administering a therapeutically effective dose of a composition comprising a bitterness reducing agent, tadalafil and sildenafil. In some embodiments, the composition is administered sublingually.

### BRIEF DESCRIPTION OF THE DRAWINGS

FIG. 1 shows a UV/visible spectrum of an NF01 bitterness reducing agent used in some embodiments of the present disclosure (solid line) with a maximum absorbance at 288 nm.

FIG. 2 shows a UV/visible spectrum of an acacia sweetener or flavoring agent used in some embodiments of the present disclosure (broken line) with a maximum absorbance at 277 nm.

FIG. 3 shows a UV/visible spectrum of a steviol sweetener or flavoring agent used in some embodiments of the present disclosure (broken line) with a maximum absorbance at 252 nm.

FIG. 4 shows a UV/visible spectrum of a spearmint oil sweetener or flavoring agent used in some embodiments of the present disclosure (broken line) with a maximum absorbance at 252 nm.

FIG. 5 shows a UV/visible spectrum of an acesulfame potassium sweetener or flavoring agent used in some embodiments of the present disclosure (broken line) with a maximum absorbance at 252 nm.

FIG. 6 shows a UV/visible spectrum of a menthol sweetener or flavoring agent in some embodiments of the present disclosure (broken line) with a maximum absorbance at 283 nm.

FIG. 7 shows a UV/visible spectrum of a silica sweetener or flavoring agent used in some embodiments of the present disclosure (broken line) with a maximum absorbance at 321 nm.

FIG. 8 shows a UV/visible spectrum of a marshmallow sweetener or flavoring agent used in some embodiments of the present disclosure (broken line) with a maximum absorbance at 277 nm.

FIG. 9 shows a UV/visible spectrum of a vanilla sweetener or flavoring agent used in some embodiments of the present disclosure (broken line) with absorption bands at 277 nm and 308 nm.

FIG. 10 shows a UV/visible spectrum of a chocolate sweetener or flavoring agent used in some embodiments of the present disclosure (broken line) with a maximum absorbance at 277 nm.

FIG. 11 is a front view of dosage form packaging according to some embodiments.

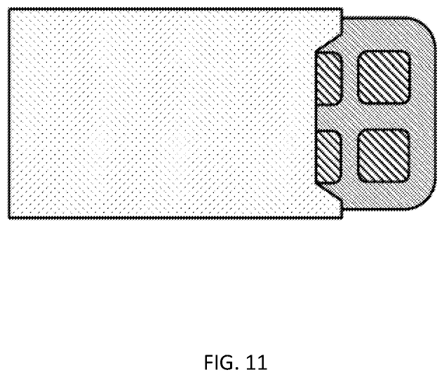

### DEFINITIONS

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “wt. %”, unless specified otherwise, refers to the weight of a component relative to total weight of the composition.

The terms “administer,” “administering” or “administration” as used herein refer to either directly administering a compound or pharmaceutically acceptable salt, solvate, prodrug, or ester of the compound or a composition comprising the compound or pharmaceutically acceptable salt, solvate, prodrug, or ester of the compound to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” as used herein means, and is used interchangeably with, the terms disease, condition, or illness, unless otherwise indicated.

The terms “effective amount” and “therapeutically effective amount” are used interchangeably in this disclosure and refer to an amount of a compound, or a salt, solvate, prodrug, or ester thereof, that, when administered to a patient, is capable of performing the intended result. For example, in some embodiments, an effective amount of sildenafil is that amount that is required to reduce at least one symptom of erectile dysfunction in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “pharmaceutically acceptable salts” includes those obtained by reacting the active compound functioning as a base, with an inorganic or organic acid to form a salt, for example, salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. Those skilled in the art will further recognize that acid addition salts may be prepared by reaction of the compounds with the appropriate inorganic or organic acid via any of a number of known methods.

The term “substantially similar” as used herein means an analytical spectrum, such as UV/visible spectrum, etc., which resembles the reference spectrum to a great degree in both the peak locations and their intensity.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, in some embodiments, the method for treating erectile dysfunction provides a therapeutic effect when the method reduces at least one symptom of erectile dysfunction in a patient.

As used herein, the term “sexual dysfunction” refers to a problem that prevents an individual or a couple from experiencing satisfaction from sexual activity.

As used herein, the term “sublingual” or “sublingual administration” refers to the pharmacological route of administration by which substances diffuse into the blood through tissues under the tongue.

As used herein, the terms “drug,” and “therapeutic agent,” refer to a compound, or a pharmaceutical composition thereof, which is administered to a subject for treating, preventing, or ameliorating one or more symptoms of a disorder.

As used herein, the terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

As used herein, the term “prodrug” refers to a compound functional derivative of the compound as disclosed herein and is readily convertible into the parent compound in vivo. The term “prodrug” denotes a compound which, upon administration to a subject, undergoes chemical conversion by metabolic or chemical processes to yield a compound of the formula, and/or a salt and/or solvate thereof. For example, compounds containing a carboxy group can form physiologically hydrolyzable esters which serve as prodrugs by being hydrolyzed in the body to yield formula compounds per se. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### DETAILED DESCRIPTION

The present disclosure provides taste-masked formulations comprising bitter active ingredients (including cannabidiol, tetrahydrocannabinol, sildenafil and combinations of sildenafil and tadalafil among other active ingredients disclosed herein) and a bitterness reducing agent.

Sublingually- or buccally-administered dosage forms have many advantages over orally administered dosage form, including rapid drug action. Nonetheless, up until the present disclosure, many FDA-approved drugs that would benefit from rapid onset after administration have not been formulated in sublingual- or buccal-dosage forms. For example, active ingredients that treat erectile dysfunction, such as sildenafil, are excellent candidates for sublingual administration because sexual activity is a frequently spontaneous rather than planned. However, the label instructions for the FDA-approved oral tablets of sildenafil instruct a patient to administer the dose approximately 1 hour before sexual activity. Sildenafil was approved by FDA in 1998, however, no FDA-approved sublingual or buccal dosage forms have been approved despite the apparent advantages of sublingual administration of the drug. It is widely understood that the bitterness associated with sildenafil is the reason there is no approved sublingual or buccal dosage form. Thus, there has been a long-felt need for sublingually- or buccally-administered dosage forms of sildenafil as well as other bitter active ingredients.

Sublingually- or buccally-administered dosage forms are also advantageous for patients who may have difficulty swallowing conventional oral dosage forms (e.g., patients with dysphagia, elderly patients, or young patients). However, as a practical matter, such dosage forms may not be acceptable for the delivery of active agents (such as those disclosed herein) that are bitter and unpalatable, as sublingual or buccal dosage forms necessarily release the active agent in the oral cavity.

Taste-masking bitter active ingredients is complex and not predictable. In addition to masking the bitter taste of the active ingredient, the formulation must be overall palatable (for example, not immediately bitter, no bitter aftertaste, not overly sweet, good mouthfeel) to provide a useful sublingual or buccal formulation of a bitter active ingredient. For example, masking bitterness by including a sweetener is often not sufficient to improve overall palatability.

The applicants have discovered that the specific selection of bitterness reducing agents and sweetener or flavoring agents was much more effective than other, similar bitterness reducing agents and sweetener or flavoring agents.

### Formulations:

The present disclosure provides taste-masked formulations comprising one or more bitter active ingredients (including sildenafil, tadalafil, tetrahydrocannabinol and cannabidiol) and a bitterness reducing agent.

The formulations of the present disclosure comprise one or more of bitter active ingredients disclosed herein (or a pharmaceutically acceptable salt, prodrug, or solvate thereof) together with one or more pharmaceutically acceptable carriers and optionally one or more other active ingredients.

The pharmaceutical compositions disclosed herein may be manufactured using methods that are known to those skilled in the art, e.g., by means of conventional mixing, dissolving, granulating, dragee making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. The dosage forms of the present disclosure may be made according to conventional methods and techniques known to those skilled in the art.

In some embodiments, the compositions of the present disclosure are formulated for buccal or sublingual administration. Exemplary buccal or sublingual include tablets, lozenges, pastilles, troches, or gels.

In some embodiments, the compositions for sublingual administration are selected from:
Sublingual tablets—tablets that easily melt in the mouth, dissolve rapidly and with little or no residue. Nitroglycerine tablets are an example, the anti-emetic ondansetron is another.
Sublingual strips—similar to tablets in that they easily melt in the mouth and dissolve rapidly. Suboxone is an example of medication that comes in a sublingual strip.
Multi-purpose tablets—Soluble tablets for either oral or sublingual (or buccal) administration, often also suitable for preparation of injections, Hydrostat (hydromorphone) and a number of brands of morphine tablets and cubes.
Sublingual drops—a concentrated solution to be dropped under the tongue, as with some nicocodeine cough preparations,
Sublingual spray—spray for the tongue; certain human and veterinary pharmaceuticals are dispensed as such.
Lozenge—effects a metered and patient-controlled-rate combination of sublingual, buccal, and oral administration, as with the Actiq fentanyl lozenge-on-a-stick (lollipop).
Effervescent buccal or sublingual tablets—this method drives the pharmaceutical through the mucous membranes much faster (this is the case in the stomach with carbonated or effervescent liquids as well) and is used in the Fentora fentanyl buccal tablet.

In some embodiments, the sublingual compositions of the present disclosure are troches. A troche comprises an active ingredient worked into a paste with sugar and mucilage or the like, and dried. In some embodiments, the troches of the present disclosure are small lozenges that dissolve under the tongue over a period of about 6 minutes to 10 minutes. As the Troche dissolves, the medication is gradually absorbed into the blood stream so that it can be absorbed more efficiently and more rapidly than if it was taken orally.

In some embodiments, the sublingual compositions of the present disclosure are selected from troches, lozenges and lollipops. In some embodiments, the troches, lozenges, and lollipops of the present disclosure comprise a bitter active ingredient and a base that when mixed with the active ingredient provides a stable dosage form that dissolves in the mouth. The base may be selected by persons skilled in the art to provide a hard, semi-hard, or paste-like dosage form.

In some embodiments, the base is a sugar and other carbohydrates (generally provides a hard dosage form). In some embodiments, the base is polyethylene glycols and other ingredients (generally provides a soft dosage form). In some embodiments, the base is a glycerin-gelatin combination (generally provides a chewable dosage form).

In some embodiments, the sublingual compositions of the present disclosure are tablet triturates. A tablet triturate is a small tablet that is made in a mold and intended for sublingual administration. It usually weighs about 30-250 mg. A tablet triturate rapidly dissolves under the tongue, is rapidly absorbed, avoids first pass through liver, and provides a rapid therapeutic response. In some embodiments, the tablet triturates of the present disclosure comprise a bitter active ingredient and a base selected lactose, sucrose, dextrose, mannitol, and the like.

In some embodiments, the tablet triturate base comprises four parts lactose and one part sucrose. In some embodiments, the tablet triturates of the present disclosure are prepared by a process comprising, triturating the powder ingredient, mixing by geometric dilution, and moistening with a solution containing four parts alcohol and one part purified water to provide a powder mixture that is adhesive and pressing the adhesive mixture into a mold.

Almost any form of a substance may be amenable to sublingual administration if it dissolves easily in saliva. Powders and aerosols may all take advantage of this method. However, a number of factors, such as pH, molecular weight, and lipid solubility, may determine whether the route is practical. Based on these properties, a suitably soluble pharmaceutical may diffuse too slowly through the mucosa to be effective. However, many pharmaceuticals are much more potent taken sublingually, and it is generally a safer alternative than administration via the nasal mucosa.

Systemic pharmaceutical delivery through the sublingual route has emerged from the desire to provide immediate onset of pharmacological effect, bypassing first pass metabolism thru the liver. In some embodiments, the sublingual delivery of the compositions of the present disclosure avoids any gastric emptying time due to the type of meal eaten and variations in GI motility from person to person. In some embodiments, the sublingual delivery of the compositions of the present disclosure provide a rapid onset of the bitter active ingredient (for example, sildenafil and/or sildenafil citrate) into the blood stream.

Dysphagia (difficulty in swallowing) is a common problem of all age groups. Sublingual administration of the pharmaceutical means placement of the pharmaceutical under the tongue, such that the pharmaceutical reaches directly in to the blood stream through the ventral surface of the tongue and floor of the mouth, and is then absorbed into the reticulated vein which lies underneath the oral mucosa, is transported through the facial veins internal jugular vein, and brachiocephalic vein and then drained in to systemic circulation. In some embodiments, the main mechanism for the absorption of the pharmaceutical in to oral mucosa is via passive diffusion into the lipoidal membrane. The absorption of the pharmaceutical through the sublingual route is 3 to 10 times greater than oral route. Absorption means transfer of pharmaceutical from its site of administration to the systemic circulation, so it is obvious that absorption is directly proportional to the membrane layer thickness.

Sublingual>Buccal>Gingival>Palatal having mucosa thickness of 100-200, 200, 250, 500-600 micrometer, respectively

In terms of permeability, the sublingual area of the oral cavity is more permeable than the buccal (cheek) area, which in turn is more permeable than the palatal (roof of the mouth) area. A small volume of saliva is usually sufficient to result in tablet disintegration in the oral cavity.

Due to high permeability and the rich blood supply, the sublingual route is capable of producing a rapid onset of action, which makes it an appropriate route for pharmaceuticals with a short delivery period and infrequent dosing regimen.

Factors affecting the sublingual absorption include, but are not limited to, lipophilicity of the pharmaceutical, solubility in salivary secretion, pH and pKa of the saliva, binding to oral mucosa, thickness of oral epithelium, and oil to water coefficient. For a pharmaceutical to be absorbed completely through the sublingual route, the pharmaceutical must have a slightly higher lipid solubility than that required for GI absorption, which is necessary for passive permeation. In addition to high lipid solubility, the pharmaceutical should be aqueous in buccal fluids. As the mean pH of the saliva is 6.0, this pH favors absorption of the pharmaceuticals, which remain unionized. Absorption of the pharmaceuticals thru the oral mucosa occurs if the pKa is greater than 2 for an acid and less than 10 for a base. Systemic availability of pharmaceuticals that bind to oral mucosa is poor. As the thickness of the sublingual epithelium is 100-200 μm, which is less as compared to buccal thickness, the absorption of the pharmaceuticals is faster due to thinner epithelium and also the immersion of pharmaceuticals in smaller volume of saliva. Compounds with favorable oil-to-water partition coefficients are readily absorbed through the oral mucosa. An oil-to-water partition coefficient range of 40-2000 is considered optimal for the pharmaceuticals to be absorbed sublingually.

Using art-recognized techniques, the troches of the present disclosure may be customized to meet the individual needs of patients.

In some embodiments, the compositions of the present disclosure comprise one or more bitter active ingredients (including sildenafil, tadalafil, tetrahydrocannabinol, and cannabidiol), and a bitterness reducing agent. In other embodiments, the compositions of the present disclosure comprise one or more of any of the bitter active ingredients disclosed herein, and a bitterness reducing agent.

In some embodiments, the compositions of the present disclosure are troches. In such embodiments, the compositions comprise a troche base. In some embodiments, the troche base is polyethylene glycol (PEG), gelatin, gelatin containing a mixture of sorbitol and glycerol, or sorbitol. In some embodiments, the PEG has a molecular weight ranging from 500 to 5000 g/mol, 1000 to 2000 g/mol, or 1300 to 1650 g/mol, inclusive of all values and ranges that fall between these values. In some embodiments, the troche base is PEG 1450, PEG 3350, PEG 850, polyPE. In some embodiments, the troche composition comprises from about 80% by weight to about 95% by weight of a troche base. In some embodiments, the troche base is present in an amount that causes the sublingual dosage form to dissolve within about 6 minute (+/−1 minute), e.g., about 5 minutes, about 5.1 minute, about 5.2 minutes, about 5.3 minutes, about 5.4 minutes, about 5.5 minutes, about 5.6 minutes, about 5.7 minutes, about 5.8 minutes, about 5.9 minutes, about 6 minutes, about 6.1 minute, about 6.2 minutes, about 6.3 minutes, about 6.4 minutes, about 6.5 minutes, about 6.6 minutes, about 6.7 minutes, about 6.8 minutes, about 6.9 minutes, or about 7.0 minutes, inclusive of all values and ranges therebetween. In some embodiments, the troche base is present in an amount that causes the sublingual dosage form to dissolve within about 6 minute (+/−1 minute) after oral administration. In some embodiments, the troche base is present in an amount that causes the sublingual dosage form to dissolve within about 6 minute (+/−1 minute) when tested in the USP <701> Disintegration Test.

In some embodiments, the bitterness reducing agent is selected from the group consisting of grapefruit extract, NF01 (available from Ferror Health Group), PCCA Bitterness Reducing Agent, Bitter Stop PCCA, BitterStop Medisca, AlphaBitterness Reducing Agent and Bitterness Suppressor Flavor, and mixtures thereof. In some embodiments, the compositions of the present disclosure comprise from about 1.0% by weight (wt. %) to about 5.0% by weight of a bitterness reducing agent, including about 1.5 wt. %, about 2.0 wt. %, about 2.5 wt. %, about 3.0 wt. %, about 3.5 wt. %, about 4.0 wt. %, about 4.5 wt. % and about 5.0 wt. %, including all ranges there between). In some embodiments, the compositions of the present disclosure comprise about 1.0 wt. %, about 1.5 wt. %, about 2.0 wt. %, about 2.5 wt. %, about 3.0 wt. %, about 3.5 wt. %, about 4.0 wt. %, about 4.5 wt. % or about 5.0 wt. %, of a bitterness reducing agent. In some embodiments, the compositions of the present disclosure comprise about 3.0 wt. % of a bitterness reducing agent.

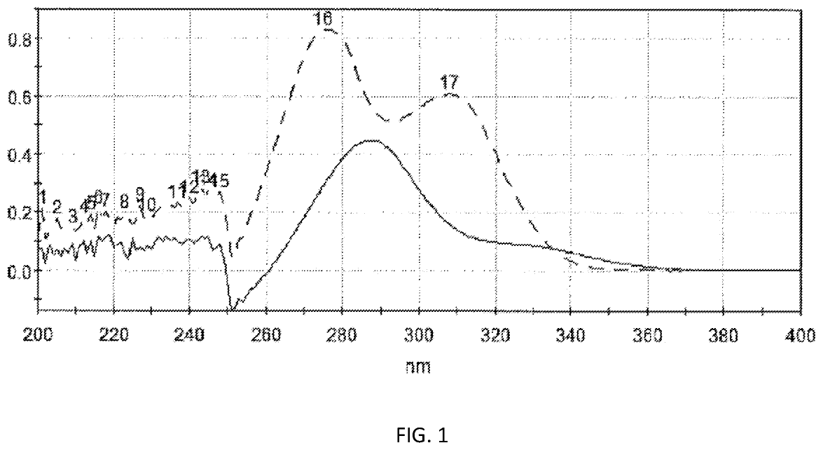

In some embodiments, the bitterness reducing agent comprises NF01 (Ferror Health Group or Medisca). In some embodiments, the NF01 exhibits a UV/visible spectrum comprising an absorption band at about 288 nm. In some embodiments, the NF01 exhibits a UV/visible spectrum comprising a maximum absorption band at about 288 nm. In some embodiments, the NF01 exhibits a UV/visible spectrum that is substantially similar to FIG. 1 (solid line). In some embodiments, the bitterness reducing agent is NF01, sold by Medisca.

In some embodiments, the compositions of the present disclosure comprise the bitterness reducing agent in the range of about 60 wt. % to about 80 wt. % (e.g., about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, about 70 wt. %, about 71 wt. %, about 72 wt. %, about 73 wt. %, about 74 wt. %, about 75 wt. %, about 76 wt. %, about 77 wt. %, about 78 wt. %, about 79 wt. %, and about 80 wt. %) with respect to the weight of the bitter active ingredient (or ingredients), and all subranges therein. In some embodiments, the compositions of the present disclosure comprise the bitterness reducing agent in about 68 wt. %, about 69 wt. %, about 70 wt. %, about 71 wt. %, or about 72 wt. % with respect to the weight of the bitter active ingredient. In some embodiments, the compositions of the present disclosure comprise the bitterness reducing agent in about 70 wt. % with respect to the weight of the bitter active ingredient when the composition comprises about 40 mg of the bitter active ingredient. In one embodiment, the bitter active ingredient is sildenafil or a pharmaceutically salt thereof. In one embodiment, when the bitter active ingredient is a pharmaceutically acceptable salt, the wt. % of the ingredient is calculated with respect to weight which is equivalent to the non-salt form of the bitter active ingredient (i.e., calculation based on 40 mg of sildenafil if 56 mg of sildenafil citrate is used in the formulation).

In some embodiments, the compositions of the present disclosure comprise the bitterness reducing agent in the range of about 40 wt. % to about 60 wt. % (e.g., about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, or about 60 wt. %) with respect to the weight of the bitter active ingredient, and all values and subranges therein. In some embodiments, the compositions of the present disclosure comprise the bitterness reducing agent in about 46 wt. %, about 47 wt. %, about 48 wt. %, about 49 wt. %, or about 50 wt. % with respect to the weight of the bitter active ingredient. In some embodiments, the compositions of the present disclosure comprise the bitterness reducing agent in about 48 wt. % with respect to the weight of the bitter active ingredient when the composition comprises about 65 mg of the bitter active ingredient.

In some embodiments, the compositions of the present disclosure comprise the bitterness reducing agent in the range of about 30 wt. % to about 50 wt. % (e.g., about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, or about 50 wt. %) with respect to the weight of the bitter active ingredient, and all subranges therein. In some embodiments, the compositions of the present disclosure comprise the bitterness reducing agent in about 38 wt. %, about 39 wt. %, about 40 wt. %, about 41 wt. %, or about 42 wt. % with respect to the weight of the bitter active ingredient. In some embodiments, the compositions of the present disclosure comprise the bitterness reducing agent in about 40 wt. % with respect to the weight of the bitter active ingredient when the composition comprises about 85 mg of the bitter active ingredient. In some embodiments, the compositions of the present disclosure comprise the bitterness reducing agent in about 39 wt. % with respect to the weight of the bitter active ingredient when the composition comprises about 110 mg of the bitter active ingredient.

In some embodiments, the compositions of the present disclosure comprise the bitterness reducing agent in the range of about 20 wt. % to about 40 wt. % (e.g., about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, or about 40 wt. %) with respect to the weight of the bitter active ingredient, and all subranges therein. In some embodiments, the compositions of the present disclosure comprise the bitterness reducing agent in about 27 wt. %, about 28 wt. %, about 29 wt. %, about 30 wt. %, or about 31 wt. % with respect to the weight of the bitter active ingredient. In some embodiments, the compositions of the present disclosure comprise the bitterness reducing agent in about 29 wt. % with respect to the weight of the bitter active ingredient when the composition comprises about 150 mg of the bitter active ingredient.

In some embodiments, the compositions of the present disclosure comprise a bitterness reducing agent ranges from about 25 mg to about 35 mg (e.g., about 25, about 26, about 27, about 28, about 29, about 30, about 31, about 32, about 33, about 34, or about 35, inclusive of all values and ranges between these values), when the composition comprises 40 mg or less of the bitter active ingredient. In some embodiments, the compositions of the present disclosure comprise about 28 mg of the bitterness reducing agent, when the composition comprises about 40 mg of the bitter active ingredient. In some embodiments, the compositions of the present disclosure comprise a bitterness reducing agent in an amount ranging from 37 mg to about 47 (e.g., about 37, about 38, about 39, about 40, about 41, about 42, about 43, about 44, about 45, about 46, or about 47, inclusive of all values and ranges between these values), when the composition comprises the bitter active ingredient in the amount ranging from 40 mg to about 80 mg. In some embodiments, the compositions of the present disclosure comprise a bitterness reducing agent in an amount ranging from 53 mg to about 63 mg (e.g., about 53, about 54, about 55, about 56, about 57, about 58, about 59, about 60, about 61, about 62, or about 63, inclusive of all values and ranges between these values), when the composition comprises the bitter active ingredient in the amount ranging from 80 mg to about 120 mg. In some embodiments, the compositions of the present disclosure comprise the bitterness reducing agent in an amount ranging from about 67 mg to about 77 mg (e.g., about 67, about 68, about 69, about 60, about 61, about 62, about 63, about 64, about 65, about 66, or about 67, inclusive of all values and ranges between these values), when the composition comprises the bitter active ingredient in the amount ranging from 120 mg to about 160 mg. In some embodiments, the compositions of the present disclosure comprise the bitterness reducing agent is present in an amount ranging from 37 mg to about 47 (e.g., about 37, about 38, about 39, about 40, about 41, about 42, about 43, about 44, about 45, about 46, or about 47, inclusive of all values and ranges between these values), when the composition comprises the bitter active ingredient in the amount ranging from 40 mg to about 150 mg. In some embodiments, the compositions of the present disclosure comprise about 31 mg to about 34 mg of the bitterness reducing agent, when the composition comprises the bitter active ingredient in the amount ranging from 65 mg to about 85 mg. In some embodiments, the compositions of the present disclosure comprise about 42 mg of the bitterness reducing agent, when the composition comprises the bitter active ingredient in the amount ranging from 110 mg to about 150 mg. In one embodiment, the bitter active ingredient is sildenafil, tadalafil, or a pharmaceutically salt thereof.

In some embodiments, the compositions further comprise a sweetener or flavoring agent. In some embodiments, the sweetener or flavoring agent selected from the group consisting of sucrose, acesulfame, cherry, Splenda®, steviol, silica, menthol (sold by PCCA), chocolate (sold by Medisca), spearmint (sold by PCCA), vanilla (sold by Flavor RX), tutti frutti, winterfresh, watermelon, butterscotch, buttercream, caramel, and marshmallow (sold by Medisca), and mixtures thereof. In some embodiments, the compositions comprise acesulfame and steviol in a ratio of about 1:1 by weight. In some embodiments, the compositions of the present disclosure comprise from about 1.0% by weight (wt. %) to about 15.0% by weight of a sweetener or flavoring agent, including about 1.0 wt. %, about 2.0 wt. %, about 3.0 wt. %, about 4.0 wt. %, about 5.0 wt. %, 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, and about 15 wt. %, including all ranges there between. In some embodiments, the compositions of the present disclosure comprise about 1.0 wt. %, about 2.0 wt. %, about 3.0 wt. %, about 4.0 wt. %, about 5.0 wt. %, 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, or about 15 wt. % of a sweetener or flavoring agent. In some embodiments, the compositions of the present disclosure comprise about 9.0 wt. % of a sweetener or flavoring agent.

In some embodiments, the compositions of the present disclosure comprise the sweetener or flavoring agent in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %) with respect to the weight of the bitter active ingredient, and all values and subranges therein. In some embodiments, the compositions of the present disclosure comprise the sweetener or flavoring agent in about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, or about 14.5 wt. % with respect to the weight of the bitter active ingredient. In some embodiments, the compositions of the present disclosure comprise he sweetener or flavoring agent in about 12.5 wt. % with respect to the weight of the bitter active ingredient, wherein the composition comprises the bitter active agent in the range of about 20 mg to about 200 mg, and all subranges therein. In some embodiments, the compositions of the present disclosure comprise in about 12.5 wt. % with respect to the weight of the bitter active ingredient, wherein the composition comprises the bitter active agent in the amount of about 40 mg, about 65 mg, about 85 mg, about 110 mg, or about 150 mg. In one embodiment, the bitter active ingredient is sildenafil or a pharmaceutically salt thereof.

In some embodiments, the compositions of the present disclosure comprise acesulfame in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %)with respect to the weight of the bitter active ingredient, and all subranges therein. In some embodiments, the compositions of the present disclosure comprise acesulfame in about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, or about 14.5 wt. % with respect to the weight of the bitter active ingredient. In some embodiments, the compositions of the present disclosure comprise acesulfame in about 12.5 wt. % with respect to the weight of the bitter active ingredient, wherein the composition comprises the bitter active agent in the range of about 20 mg to about 200 mg, and all subranges therein. In some embodiments, the compositions of the present disclosure comprise acesulfame in about 12.5 wt. % with respect to the weight of the bitter active ingredient, wherein the composition comprises the bitter active agent in the amount of about 40 mg, about 65 mg, about 85 mg, about 110 mg, or about 150 mg. In one embodiment, the bitter active ingredient is sildenafil or a pharmaceutically salt thereof. In one embodiment, acesulfame is acesulfame potassium powder.

In some embodiments, the compositions of the present disclosure comprise steviol in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %) with respect to the weight of the bitter active ingredient, and all subranges therein. In some embodiments, the compositions of the present disclosure comprise steviol in about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, or about 14.5 wt. % with respect to the weight of the bitter active ingredient. In some embodiments, the compositions of the present disclosure comprise steviol in about 12.5 wt. % with respect to the weight of the bitter active ingredient, wherein the composition comprises the bitter active agent in the range of about 20 mg to about 200 mg, and all subranges therein. In some embodiments, the compositions of the present disclosure comprise steviol in about 12.5 wt. % with respect to the weight of the bitter active ingredient, wherein the composition comprises the bitter active agent in the amount of about 40 mg, about 65 mg, about 85 mg, about 110 mg, or about 150 mg. In one embodiment, the bitter active ingredient is sildenafil or a pharmaceutically salt thereof. In one embodiment, steviol is steviol glycosides. In one embodiment, steviol is steviol glycosides 95% (powder).

In some embodiments, the compositions of the present disclosure comprise acacia in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %) with respect to the weight of the bitter active ingredient, and all subranges therein. In some embodiments, the compositions of the present disclosure comprise acacia in about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, or about 14.5 wt. % with respect to the weight of the bitter active ingredient. In some embodiments, the compositions of the present disclosure comprise acacia in about 12.5 wt. % with respect to the weight of the bitter active ingredient, wherein the composition comprises the bitter active agent in the range of about 20 mg to about 200 mg, and all subranges therein. In some embodiments, the compositions of the present disclosure comprise acacia in about 12.5 wt. % with respect to the weight of the bitter active ingredient, wherein the composition comprises the bitter active agent in the amount of about 40 mg, about 65 mg, about 85 mg, about 110 mg, or about 150 mg. In one embodiment, the bitter active ingredient is sildenafil or a pharmaceutically salt thereof. In one embodiment, acacia is acacia NF. In one embodiment, acacia is acacia NF which is a spray dried gum Arabic.

In some embodiments, the compositions of the present disclosure comprise silica in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %) with respect to the weight of the bitter active ingredient, and all subranges therein. In some embodiments, the compositions of the present disclosure comprise silica in about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, or about 14.5 wt. % with respect to the weight of the bitter active ingredient. In some embodiments, the compositions of the present disclosure comprise silica in about 12.5 wt. % with respect to the weight of the bitter active ingredient, wherein the composition comprises the bitter active agent in the range of about 20 mg to about 200 mg, and all subranges therein. In some embodiments, the compositions of the present disclosure comprise silica in about 12.5 wt. % with respect to the weight of the bitter active ingredient, wherein the composition comprises the bitter active agent in the amount of about 40 mg, about 65 mg, about 85 mg, about 110 mg, or about 150 mg. In one embodiment, the bitter active ingredient is sildenafil or a pharmaceutically salt thereof. In one embodiment, silica is micornized powder silica gel.

In some embodiments, the compositions of the present disclosure comprise the sweetener or flavoring agent in the range of about 5 wt. % to about 35 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, or about 35 wt. %) with respect to the weight of the bitter active ingredient, and all subranges therein. In some embodiments, the compositions of the present disclosure comprise the bitter active agent in the amount of about 40 mg, about 65 mg, about 85 mg, about 110 mg, or about 150 mg. In one embodiment, the bitter active ingredient is sildenafil or a pharmaceutically salt thereof.

In some embodiments, the compositions of the present disclosure comprise spearmint oil in the range of about 15 wt. % to about 35 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, or about 35 wt. %) with respect to the weight of the bitter active ingredient, and all subranges therein. In some embodiments, the compositions of the present disclosure comprises spearmint oil in about 21 wt. %, about 22 wt. %, about 23 wt. %, about 24 wt. %, or about 25 wt. % with respect to the weight of the bitter active ingredient. In some embodiments, the compositions of the present disclosure comprise spearmint oil in about 23 wt. % with respect to the weight of the bitter active ingredient when the composition comprises about 40 mg of the bitter active ingredient. In one embodiment, the bitter active ingredient is sildenafil or a pharmaceutically salt thereof.

In some embodiments, the compositions of the present disclosure comprise spearmint oil in the range of about 5 wt. % to about 20 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, or about 35 wt. %) with respect to the weight of the bitter active ingredient, and all subranges therein. In some embodiments, the compositions of the present disclosure comprises spearmint oil in about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, or about 20 wt. %, with respect to the weight of the bitter active ingredient. In some embodiments, the compositions of the present disclosure comprise spearmint oil in about 14 wt. % with respect to the weight of the bitter active ingredient when the composition comprises about 65 mg of the bitter active ingredient. In some embodiments, the compositions of the present disclosure comprise spearmint oil in about 13 wt. % with respect to the weight of the bitter active ingredient when the composition comprises about 85 mg of the bitter active ingredient. In some embodiments, the compositions of the present disclosure comprise spearmint oil in about 11 wt. % with respect to the weight of the bitter active ingredient when the composition comprises about 110 mg of the bitter active ingredient. In some embodiments, the compositions of the present disclosure comprise spearmint oil in about 8 wt. % with respect to the weight of the bitter active ingredient when the composition comprises about 150 mg of the bitter active ingredient. In one embodiment, the bitter active ingredient is sildenafil or a pharmaceutically salt thereof.

In some embodiments, the compositions of the present disclosure comprise the sweetener or flavoring agent in the range of about 25 wt. % to about 75 wt. % with respect to the weight of the bitter active ingredient, and all subranges therein. In some embodiments, the compositions of the present disclosure comprise the bitter active agent in the amount of about 40 mg, about 65 mg, about 85 mg, about 110 mg, or about 150 mg. In one embodiment, the bitter active ingredient is sildenafil or a pharmaceutically salt thereof.

In some embodiments, the compositions of the present disclosure comprise marshmallow liquid in the range of about 55 wt. % to about 75 wt. % (e.g., about 55 wt. %, about 56 wt. %, about 57 wt. %, about 58 wt. %, about 59 wt. %, about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, about 70 wt. %, about 71 wt. %, about 72 wt. %, about 73 wt. %, about 74 wt. %, or about 75 wt. %) with respect to the weight of the bitter active ingredient, and all subranges therein. In some embodiments, the compositions of the present disclosure comprises marshmallow liquid in about 66 wt. %, about 67 wt. %, about 68 wt. %, about 69 wt. %, or about 70 wt. % with respect to the weight of the bitter active ingredient. In some embodiments, the compositions of the present disclosure comprise marshmallow liquid in about 68 wt. % with respect to the weight of the bitter active ingredient when the composition comprises about 40 mg of the bitter active ingredient. In one embodiment, the bitter active ingredient is sildenafil or a pharmaceutically salt thereof.

In some embodiments, the compositions of the present disclosure comprise marshmallow liquid in the range of about 45 wt. % to about 65 wt. % (e.g., about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, about 60 wt. %, about 61 wt. %, about 62 wt. %, about 63 wt. %, about 64 wt. %, or about 65 wt. %) with respect to the weight of the bitter active ingredient, and all subranges therein. In some embodiments, the compositions of the present disclosure comprises marshmallow liquid in about 54 wt. %, about 55 wt. %, about 56 wt. %, about 57 wt. %, or about 58 wt. % with respect to the weight of the bitter active ingredient. In some embodiments, the compositions of the present disclosure comprise marshmallow liquid in about 56 wt. % with respect to the weight of the bitter active ingredient when the composition comprises about 65 mg of the bitter active ingredient. In one embodiment, the bitter active ingredient is sildenafil or a pharmaceutically salt thereof.

In some embodiments, the compositions of the present disclosure comprise marshmallow liquid in the range of about 30 wt. % to about 50 wt. % (e.g., about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, or about 41 wt. %, about 42 wt. %, about 43 wt. %, about 44 wt. %, about 45 wt. %, about 46 wt. %, about 47 wt. %, about 48 wt. %, about 49 wt. %, or about 50 wt. %) with respect to the weight of the bitter active ingredient, and all subranges therein. In some embodiments, the compositions of the present disclosure comprises marshmallow liquid in about 39 wt. %, about 40 wt. %, about 41 wt. %, about 42 wt. %, about 43 wt. %, about 44 wt. %, or about 45 wt. % with respect to the weight of the bitter active ingredient. In some embodiments, the compositions of the present disclosure comprise marshmallow liquid in about 43 wt. % with respect to the weight of the bitter active ingredient when the composition comprises about 85 mg of the bitter active ingredient. In some embodiments, the compositions of the present disclosure comprise marshmallow liquid in about 41 wt. % with respect to the weight of the bitter active ingredient when the composition comprises about 110 mg of the bitter active ingredient. In one embodiment, the bitter active ingredient is sildenafil or a pharmaceutically salt thereof.

In some embodiments, the compositions of the present disclosure comprise marshmallow liquid in the range of about 20 wt. % to about 40 wt. % (e.g., about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, or about 40 wt. %) with respect to the weight of the bitter active ingredient, and all subranges therein. In some embodiments, the compositions of the present disclosure comprises marshmallow liquid in about 28 wt. %, about 29 wt. %, about 30 wt. %, about 31 wt. %, or about 32 wt. % with respect to the weight of the bitter active ingredient. In some embodiments, the compositions of the present disclosure comprise marshmallow liquid in about 30 wt. % with respect to the weight of the bitter active ingredient when the composition comprises about 150 mg of the bitter active ingredient. In one embodiment, the bitter active ingredient is sildenafil or a pharmaceutically salt thereof.

In some embodiments, the compositions of the present disclosure comprise 0.029 mL ±10% (0.031±10%) of the marshmallow liquid, when the composition comprises 40 mg or less of the bitter active ingredient. In some embodiments, the compositions of the present disclosure comprise about 0.026 mL ±10% (0.028±10%) of the marshmallow liquid, when the composition comprises about 40 mg of the bitter active ingredient. In some embodiments, the compositions of the present disclosure comprise 0.043 mL ±10% (0.045±10%) of the marshmallow liquid, when the composition comprises the bitter active ingredient in the amount ranging from 40 mg to about 90 mg. In some embodiments, the compositions of the present disclosure comprise about 0.034 mL ±10% (0.036±10%) of the marshmallow liquid, when the composition comprises about 65 mg to about 85 mg of the bitter active ingredient. In some embodiments, the compositions of the present disclosure comprise 0.057 mL ±10% (0.06±10%) of the marshmallow liquid, when the composition comprises the bitter active ingredient in the amount ranging from 90 mg to about 140 mg. In some embodiments, the compositions of the present disclosure comprise 0.71 mL ±10% (0.75±10%) of the marshmallow liquid, when the composition comprises the bitter active ingredient in the amount ranging from 120 mg to about 160 mg. In some embodiments, the compositions of the present disclosure comprise about 0.043 mL ±10% (0.045±10%) of the marshmallow liquid, when the composition comprises about 110 mg to about 150 mg of the bitter active ingredient. In one embodiment, the bitter active ingredient is sildenafil, tadalafil, or a pharmaceutically salt thereof.

In some embodiments, the compositions of the present disclosure comprise vanilla liquid in the range of about 5 wt. % to about 25 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, or about 25 wt.) with respect to the weight of the bitter active ingredient, and all subranges therein. In some embodiments, the compositions of the present disclosure comprises vanilla liquid in about 13 wt. %, about 14 wt. %, about 15 wt. %, about 16 wt. %, or about 17 wt. % with respect to the weight of the bitter active ingredient. In some embodiments, the compositions of the present disclosure comprise vanilla liquid in about 15 wt. % with respect to the weight of the bitter active ingredient when the composition comprises about 40 mg of the bitter active ingredient. In one embodiment, the bitter active ingredient is sildenafil or a pharmaceutically salt thereof.

In some embodiments, the compositions of the present disclosure comprise vanilla liquid in the range of about 5 wt. % to about 15 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, or about 15 wt. %) with respect to the weight of the bitter active ingredient, and all subranges therein. In some embodiments, the compositions of the present disclosure comprises vanilla liquid in about 5 wt. %, about 6 wt. %, about 7 wt. %, about 8 wt. %, about 9 wt. %, about 10 wt. %, about 11 wt. %, about 12 wt. %, or about 13 wt. % with respect to the weight of the bitter active ingredient. In some embodiments, the compositions of the present disclosure comprise vanilla liquid in about 9 wt. % with respect to the weight of the bitter active ingredient when the composition comprises about 65 mg of the bitter active ingredient. In some embodiments, the compositions of the present disclosure comprise vanilla liquid in about 7 wt. % with respect to the weight of the bitter active ingredient when the composition comprises about 85 mg of the bitter active ingredient. In some embodiments, the compositions of the present disclosure comprise vanilla liquid in about 11 wt. % with respect to the weight of the bitter active ingredient when the composition comprises about 110 mg of the bitter active ingredient. In some embodiments, the compositions of the present disclosure comprise vanilla liquid in about 8 wt. % with respect to the weight of the bitter active ingredient when the composition comprises about 150 mg of the bitter active ingredient. In one embodiment, the bitter active ingredient is sildenafil or a pharmaceutically salt thereof.

In some embodiments, the compositions of the present disclosure comprise chocolate liquid in the range of about 55 wt. % to about 75 wt. % (e.g., about 55 wt. %, about 56 wt. %, about 57 wt. %, about 58 wt. %, about 59 wt. %, about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, about 70 wt. %, about 71 wt. %, about 72 wt. %, about 73 wt. %, about 74 wt. %, or about 75 wt. %) with respect to the weight of the bitter active ingredient, and all subranges therein. In some embodiments, the compositions of the present disclosure comprises chocolate liquid in about 65 wt. %, about 66 wt. %, about 67 wt. %, about 68 wt. %, about 69 wt. %, or about 70 wt. % with respect to the weight of the bitter active ingredient. In some embodiments, the compositions of the present disclosure comprise chocolate liquid in about 67 wt. % with respect to the weight of the bitter active ingredient when the composition comprises about 40 mg of the bitter active ingredient. In one embodiment, the bitter active ingredient is sildenafil or a pharmaceutically salt thereof.

In some embodiments, the compositions of the present disclosure comprise chocolate liquid in the range of about 40 wt. % to about 60 wt. % (e.g., about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, and about 60 wt. %) with respect to the weight of the bitter active ingredient, and all subranges therein. In some embodiments, the compositions of the present disclosure comprises chocolate liquid in about 48 wt. %, about 49 wt. %, about 50 wt. %, about 51 wt. %, or about 52 wt. % with respect to the weight of the bitter active ingredient. In some embodiments, the compositions of the present disclosure comprise chocolate liquid in about 50 wt. % with respect to the weight of the bitter active ingredient when the composition comprises about 65 mg of the bitter active ingredient. In one embodiment, the bitter active ingredient is sildenafil or a pharmaceutically salt thereof.

In some embodiments, the compositions of the present disclosure comprise chocolate liquid in the range of about 30 wt. % to about 50 wt. % (e.g., about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, or about 50 wt. %) with respect to the weight of the bitter active ingredient, and all subranges therein. In some embodiments, the compositions of the present disclosure comprises chocolate liquid in about 36 wt. %, about 37 wt. %, about 38 wt. %, about 39 wt. %, about 40 wt. %, about 41 wt. %, about 42 wt. %, about 43 wt. %, about 44 wt. %, or about 45 wt. % with respect to the weight of the bitter active ingredient. In some embodiments, the compositions of the present disclosure comprise chocolate liquid in about 38 wt. % with respect to the weight of the bitter active ingredient when the composition comprises about 85 mg of the bitter active ingredient. In some embodiments, the compositions of the present disclosure comprise chocolate liquid in about 41 wt. % with respect to the weight of the bitter active ingredient when the composition comprises about 110 mg of the bitter active ingredient. In one embodiment, the bitter active ingredient is sildenafil or a pharmaceutically salt thereof.

In some embodiments, the compositions of the present disclosure comprise chocolate liquid in the range of about 20 wt. % to about 40 wt. % (e.g., about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, or about 40 wt. %) with respect to the weight of the bitter active ingredient, and all subranges therein. In some embodiments, the compositions of the present disclosure comprises chocolate liquid in about 28 wt. %, about 29 wt. %, about 30 wt. %, about 31 wt. %, or about 32 wt. % with respect to the weight of the bitter active ingredient. In some embodiments, the compositions of the present disclosure comprise chocolate liquid in about 30 wt. % with respect to the weight of the bitter active ingredient when the composition comprises about 150 mg of the bitter active ingredient. In one embodiment, the bitter active ingredient is sildenafil or a pharmaceutically salt thereof.

In some embodiments, the compositions of the present disclosure comprise the chocolate liquid at about 0.029 mL ±10% (or about 0.03 mg ±10%), when the composition comprises 40 mg or less of the bitter active ingredient. In some embodiments, the compositions of the present disclosure comprise about 0.026 mL ±10% (0.027 mg ±10%) of the chocolate liquid, when the composition comprises about 40 mg or less of the bitter active ingredient. In some embodiments, the compositions of the present disclosure comprise 0.043 mL ±10% (0.045 mg ±10%) the chocolate liquid, when the composition comprises the bitter active ingredient in the amount ranging from 40 mg to about 90 mg. In some embodiments, the compositions of the present disclosure comprise about 0.031 mL ±10% (0.032 mg ±10%) of the chocolate liquid, when the composition comprises about 65 mg to about 85 mg of the bitter active ingredient. In some embodiments, the compositions of the present disclosure comprise 0.057 mL ±10% (0.059 mg ±10%) of the chocolate liquid, when the composition comprises the bitter active ingredient in the amount ranging from 90 mg to about 140 mg. In some embodiments, the compositions of the present disclosure comprise 0.71 mL ±10% (0.74 mg ±10%) of the chocolate liquid, when the composition comprises the bitter active ingredient in the amount ranging from 120 mg to about 160 mg. In some embodiments, the compositions of the present disclosure comprise about 0.043 mL ±10% (0.045 mg ±10%) of the chocolate liquid, when the composition comprises about 110 mg to about 150 mg of the bitter active ingredient. In one embodiment, the bitter active ingredient is sildenafil, tadalafil, or a pharmaceutically salt thereof.

In some embodiments, the compositions of the present disclosure comprise the chocolate liquid and the marshmallow liquid in about a 1:1 ratio, about1.1:1, about 1.2:1, about 1.3:1, about 1.4:1, about 1.5:1, about 1:1.1: about 1:1.2, about 1:1.3, about 1:1.4, about 1:1.5, and any values and ranges between these ratios. In some embodiments, the compositions of the present disclosure comprise the chocolate liquid and the marshmallow liquid in about a 1:1 ratio.

In some embodiments, the compositions of the present disclosure comprise the sweetener or flavoring agent in the range of about 2 wt. % to about 10 wt. % with respect to the weight of the bitter active ingredient, and all subranges therein. In some embodiments, the compositions of the present disclosure comprise the bitter active agent in the amount of about 40 mg, about 65 mg, about 85 mg, about 110 mg, or about 150 mg. In one embodiment, the bitter active ingredient is sildenafil or a pharmaceutically salt thereof.

In some embodiments, the compositions of the present disclosure comprise menthol in the range of about 2 wt. % to about 10 wt. % (e.g., about 2 wt. %, about 3 wt %, about 4 wt. %, about 5 wt. %, about 6 wt. %, about 7 wt. %, about 8 wt. %, about 9 wt. %, or about 10 wt. %) with respect to the weight of the bitter active ingredient, and all subranges therein. In some embodiments, the compositions of the present disclosure comprises menthol in about 2 wt. %, about 3 wt. %, about 4 wt. %, about 5 wt. %, about 6 wt. %, about 7 wt. %, about 8 wt. %, about 9 wt. %, or about 10 wt. % with respect to the weight of the bitter active ingredient. In some embodiments, the compositions of the present disclosure comprise menthol in about 7 wt. % with respect to the weight of the bitter active ingredient when the composition comprises about 40 mg of the bitter active ingredient. In some embodiments, the compositions of the present disclosure comprise menthol in about 5 wt. % with respect to the weight of the bitter active ingredient when the composition comprises the bitter active ingredient in the range of about 60 mg to about 120 mg. In some embodiments, the compositions of the present disclosure comprise menthol in about 5 wt. % with respect to the weight of the bitter active ingredient when the composition comprises about 65 mg, about 85 mg, or about 110 mg of the bitter active ingredient. In some embodiments, the compositions of the present disclosure comprise menthol in about 4 wt. % with respect to the weight of the bitter active ingredient when the composition comprises about 150 mg of the bitter active ingredient. In one embodiment, the bitter active ingredient is sildenafil or a pharmaceutically salt thereof.

In some embodiments, the compositions of the present disclosure comprise 2.9 mg ±10% of the menthol, when the composition comprises 60 mg or less of the bitter active ingredient. In some embodiments, the compositions of the present disclosure comprise about 2.9 mg ±10% of the menthol, when the composition comprises about 40 mg of the bitter active ingredient. In some embodiments, the compositions of the present disclosure comprise 5.7 mg ±10% of the menthol, when the composition comprises the bitter active ingredient in the amount ranging from 60 mg to about 120 mg. In some embodiments, the compositions of the present disclosure comprise about 2.9 mg ±10% of the menthol, when the composition comprises about 65 mg of the bitter active ingredient. In some embodiments, the compositions of the present disclosure comprise about 4.6 mg ±10% of the menthol, when the composition comprises about 85 mg of the bitter active ingredient. In some embodiments, the compositions of the present disclosure comprise about 5.7 mg ±10% of the menthol, when the composition comprises about 110 mg of the bitter active ingredient. In some embodiments, the compositions of the present disclosure comprise 8.6 mg ±10% of the menthol, when the composition comprises the bitter active ingredient in the amount ranging from 120 mg to about 150 mg. In some embodiments, the compositions of the present disclosure comprise about 5.7 mg ±10% of the menthol, when the composition comprises about 150 mg of the bitter active ingredient. In one embodiment, the bitter active ingredient is sildenafil, tadalafil, or a pharmaceutically salt thereof.

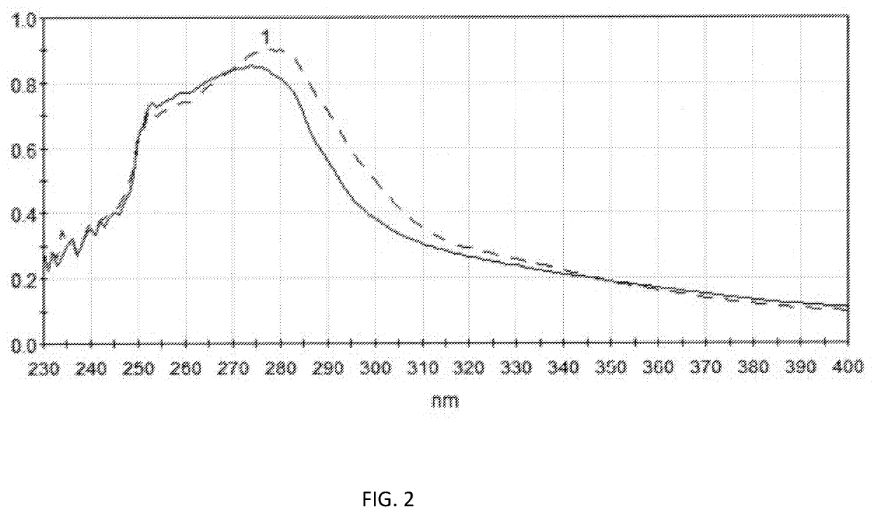

In some embodiments, the sweetener or flavoring agent comprises acacia. In some embodiments, the acacia exhibits a UV/visible spectrum comprising an absorption band at about 277 nm. In some embodiments, the acacia exhibits a UV/visible spectrum comprising a maximum absorption band at about 277 nm. In some embodiments, the acacia exhibits a UV/visible spectrum that is substantially similar to FIG. 2 (broken line).

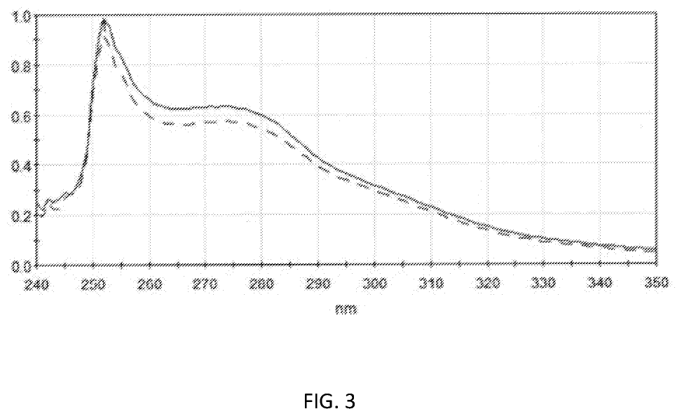

In some embodiments, the sweetener or flavoring agent comprises steviol. In some embodiments, the steviol exhibits a UV/visible spectrum comprising an absorption band at about 252 nm. In some embodiments, the steviol exhibits a UV/visible spectrum comprising a maximum absorption band at about 252 nm. In some embodiments, the steviol exhibits a UV/visible spectrum that is substantially similar to FIG. 3 (broken line).

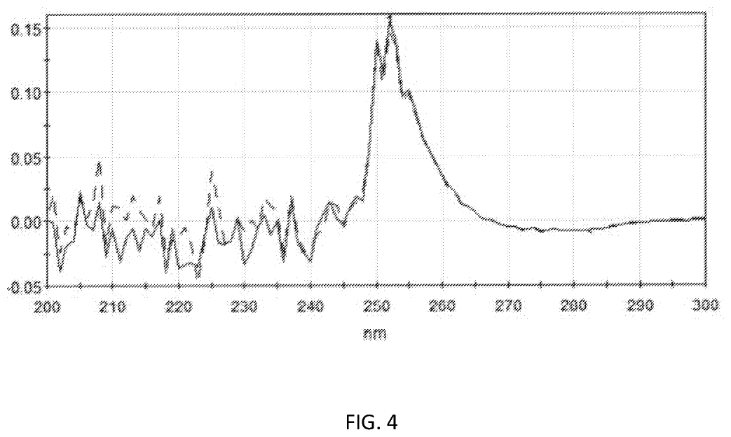

In some embodiments, the sweetener or flavoring agent comprises spearmint oil. In some embodiments, the spearmint oil exhibits a UV/visible spectrum comprising an absorption band at about 252 nm. In some embodiments, the spearmint oil exhibits a UV/visible spectrum comprising a maximum absorption band at about 252 nm. In some embodiments, the spearmint oil exhibits a UV/visible spectrum that is substantially similar to FIG. 4 (broken line).

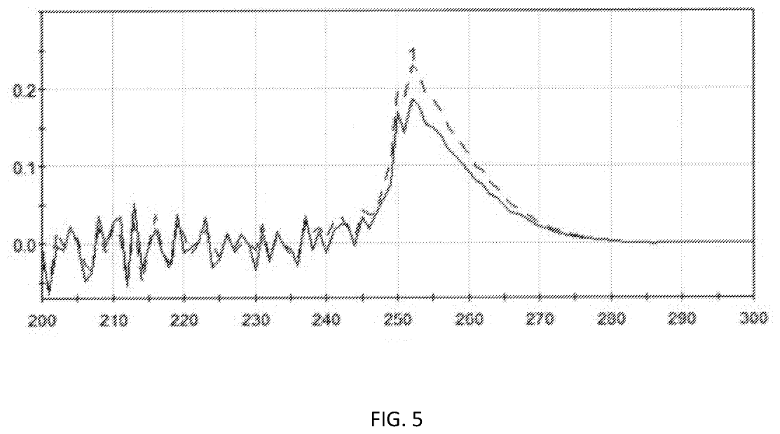

In some embodiments, the sweetener or flavoring agent comprises acesulfame. In some embodiments, the sweetener or flavoring agent comprises acesulfame potassium. In some embodiments, the acesulfame potassium exhibits a UV/visible spectrum comprising an absorption band at about 252 nm. In some embodiments, the acesulfame potassium exhibits a UV/visible spectrum comprising a maximum absorption band at about 252 nm. In some embodiments, the acesulfame potassium exhibits a UV/visible spectrum that is substantially similar to FIG. 5 (broken line).

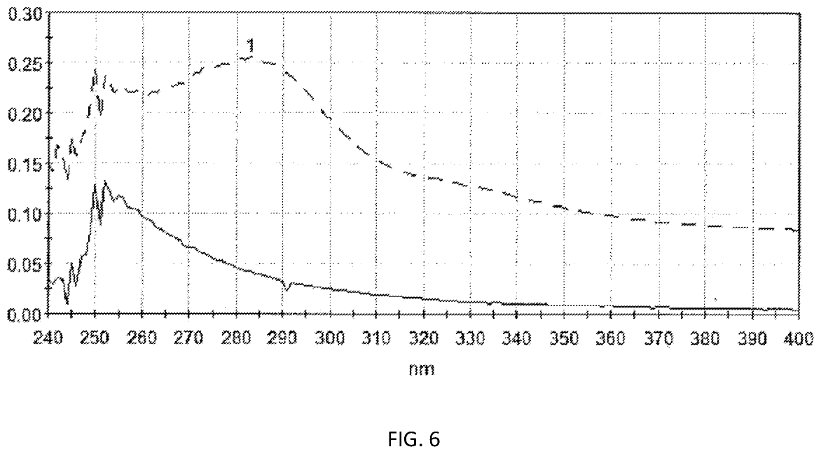

In some embodiments, the sweetener or flavoring agent comprises menthol. In some embodiments, the menthol exhibits a UV/visible spectrum comprising an absorption band at about 283 nm. In some embodiments, the menthol exhibits a UV/visible spectrum comprising a maximum absorption band at about 283 nm. In some embodiments, the menthol exhibits a UV/visible spectrum that is substantially similar to FIG. 6 (broken line).

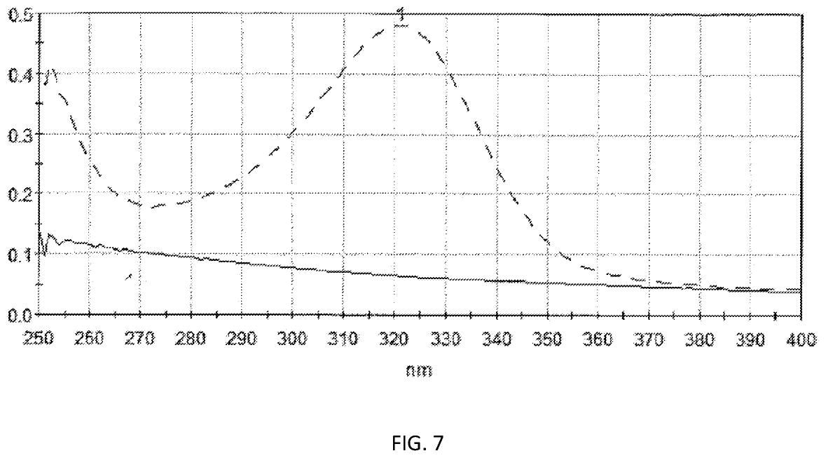

In some embodiments, the sweetener or flavoring agent comprises silica. In some embodiments, the silica exhibits a UV/visible spectrum comprising an absorption band at about 321 nm. In some embodiments, the silica exhibits a UV/visible spectrum comprising a maximum absorption band at about 321 nm. In some embodiments, the silica exhibits a UV/visible spectrum that is substantially similar to FIG. 7 (broken line).

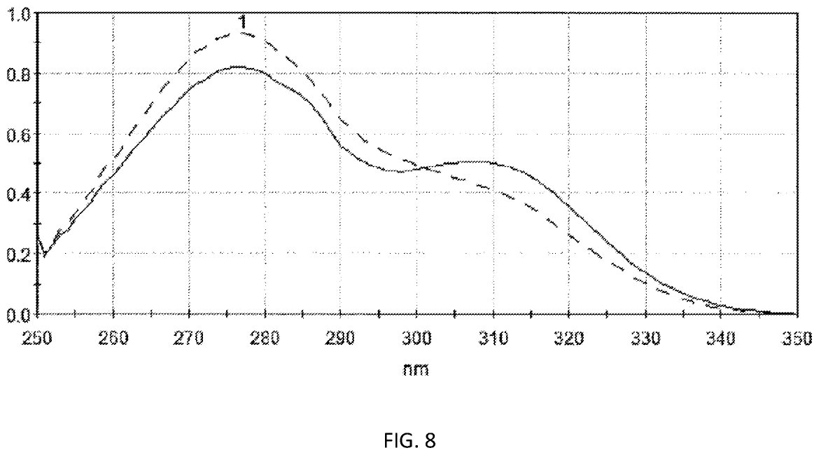

In some embodiments, the sweetener or flavoring agent comprises marshmallow. In some embodiments, the marshmallow exhibits a UV/visible spectrum comprising an absorption band at about 277 nm. In some embodiments, the marshmallow exhibits a UV/visible spectrum comprising a maximum absorption band at about 277 nm. In some embodiments, the marshmallow exhibits a UV/visible spectrum that is substantially similar to FIG. 8 (broken line).

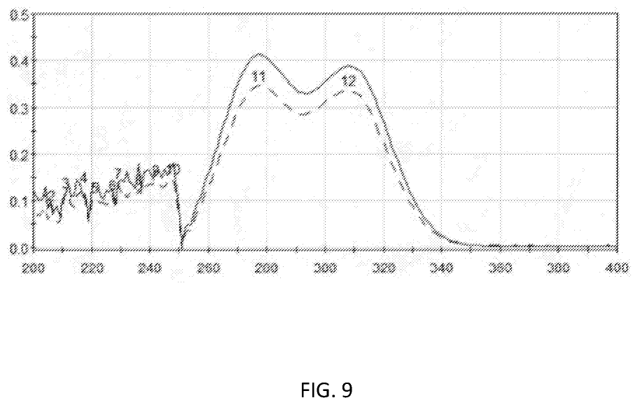

In some embodiments, the sweetener or flavoring agent comprises vanilla. In some embodiments, the vanilla exhibits a UV/visible spectrum comprising absorption bands at about 277 nm and about 308 nm. In some embodiments, the vanilla exhibits a UV/visible spectrum comprising a maximum absorption band at about 277 nm. In some embodiments, the vanilla exhibits a UV/visible spectrum that is substantially similar to FIG. 9 (broken line).

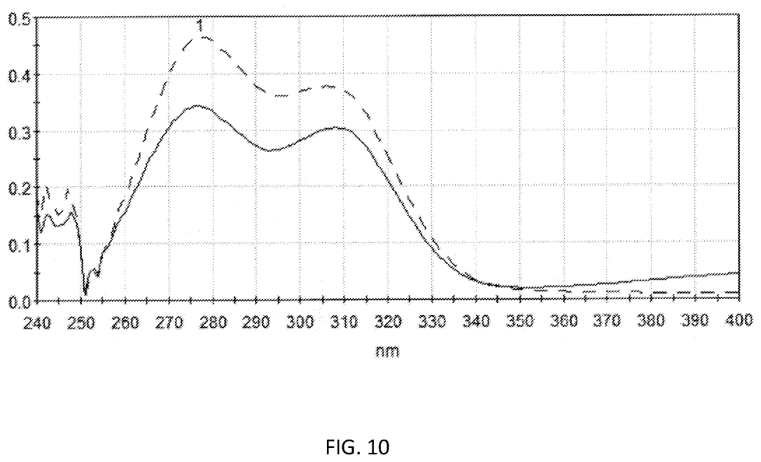

In some embodiments, the sweetener or flavoring agent comprises chocolate. In some embodiments, the chocolate exhibits a UV/visible spectrum comprising an absorption band at about 277 nm. In some embodiments, the chocolate comprises vanillin and exhibits a UV/visible spectrum comprising an absorption band at about 277 nm. In some embodiments, the chocolate comprises vanillin, butyraldehyde and phenol and exhibits a UV/visible spectrum comprising an absorption band at about 277 nm. In some embodiments, the chocolate comprises vanillin and exhibits a UV/visible spectrum that is substantially similar to FIG. 10 (broken line). In some embodiments, the chocolate comprises vanillin, butyraldehyde and phenol and exhibits a UV/visible spectrum that is substantially similar to FIG. 10 (broken line).

In some embodiments, the bitterness reducing agent comprises: NF01, wherein the NF01 exhibits a UV/visible spectrum that is substantially similar to FIG. 1 (solid line); and the sweetener or flavoring agent comprises:
acacia, wherein the acacia exhibits a UV/visible spectrum that is substantially similar to FIG. 2 (broken line);
steviol, wherein the steviol exhibits a UV/visible spectrum that is substantially similar to FIG. 3 (broken line);
spearmint oil, wherein the spearmint oil exhibits a UV/visible spectrum that is substantially similar to FIG. 4 (broken line);
acesulfame potassium, wherein the acesulfame potassium exhibits a UV/visible spectrum that is substantially similar to FIG. 5 (broken line);
menthol, wherein the menthol exhibits a UV/visible spectrum that is substantially similar to FIG. 6 (broken line);
silica, wherein the silica exhibits a UV/visible spectrum that is substantially similar to FIG. 7 (broken line);
marshmallow, wherein the marshmallow exhibits a UV/visible spectrum that is substantially similar to FIG. 8 (broken line);
vanilla, wherein the vanilla exhibits a UV/visible spectrum that is substantially similar to FIG. 9 (broken line); and
chocolate, wherein the chocolate comprises vanillin and exhibits a UV/visible spectrum that is substantially similar to FIG. 10 (broken line).

In some embodiments, the bitterness reducing agent comprises: NF01, wherein the NF01 exhibits a UV/visible spectrum comprising an absorption band at about 288 nm; and the sweetener or flavoring agent comprises:
acacia, wherein the acacia exhibits a UV/visible spectrum comprising an absorption band at about 277 nm;
steviol, wherein the steviol exhibits a UV/visible spectrum comprising an absorption band at about 252 nm;
spearmint oil, wherein the spearmint oil exhibits a UV/visible spectrum comprising an absorption band at about 252 nm;
acesulfame potassium, wherein the acesulfame potassium exhibits a UV/visible spectrum comprising an absorption band at about 252 nm;
menthol, wherein the menthol exhibits a UV/visible spectrum comprising an absorption band at about 283 nm;
silica, wherein the silica exhibits a UV/visible spectrum comprising an absorption band at about 321 nm;
marshmallow, wherein the marshmallow exhibits a UV/visible spectrum comprising an absorption band at about 277 nm;
vanilla, wherein the vanilla exhibits a UV/visible spectrum comprising absorption bands at about 277 nm and about 308; and
chocolate, wherein the chocolate exhibits a UV/visible spectrum comprising an absorption band at about 277 nm.

In some embodiments, the composition comprises:
about 2 wt. % to about 6 wt. % of a bitter active ingredient described herein,
about 1 wt. % to about 5 wt. % of a bitterness reducing agent; and
about 6 wt. % to about 10 wt. % of a sweetener or flavoring agent.

In some embodiments, the composition comprises:
about 3 wt. % to about 5 wt. % of a bitter active ingredient described herein,
about 2 wt. % to about 4 wt. % of a bitterness reducing agent; and
about 7 wt. % to about 9 wt. % of a sweetener or flavoring agent.

In some embodiments, the composition comprises:
about 4 wt. % of a bitter active ingredient described herein,
about 3 wt. % of a bitterness reducing agent; and
about 8 wt. % of a sweetener or flavoring agent.

In some embodiments, the composition comprises:
about 3 wt. % to about 5 wt. % of a bitter active ingredient described herein,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of silica,
about 0.25 wt % to about 0.75 wt. % of acacia,
about 1 wt % to about 4 wt. % of chocolate,
about 1 wt % to about 4 wt. % of marshmallow,
about 0.4 wt % to about 0.8 wt. % of vanilla and
about 0.5 wt % to about 1.5 wt. % of spearmint.

In some embodiments, the composition comprises:
about 4 wt. % of a bitter active ingredient described herein,
about 3 wt. % NF01,
about 0.5 wt. % acesulfame,
about 0.5 wt. % steviol,
about 0.5 wt. % silica,
about 0.5 wt. % acacia,
about 3 wt. % chocolate,
about 3 wt. % marshmallow,
about 0.6 wt. % vanilla and
about 1 wt. % spearmint.

In some embodiments, the bitter active ingredient is sildenafil or a pharmaceutically acceptable salt thereof. In some embodiment, the compositions comprise sildenafil citrate. The sublingual and buccal sildenafil-containing compositions of the present disclosure provide a faster as well as a more predictable onset of action compared to orally administered sildenafil. Furthermore, the rapid onset of action is not related to meal status (i.e., there is no food effect).

In some embodiments, the compositions comprise from about 5 mg to about 150 mg of sildenafil or a pharmaceutically acceptable salt thereof, including about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg and about 150 mg including all ranges there between.

In some embodiments, the compositions comprise about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg and about 150 mg of sildenafil or a pharmaceutically acceptable salt thereof. In some embodiments, the compositions of the present disclosure comprise about 20 mg of sildenafil or a pharmaceutically acceptable salt thereof. In some embodiments, the compositions of the present disclosure comprise about 40 mg of sildenafil or a pharmaceutically acceptable salt thereof. In some embodiments, the compositions of the present disclosure comprise about 65 mg of sildenafil or a pharmaceutically acceptable salt thereof. In some embodiments, the compositions of the present disclosure comprise about 80 mg of sildenafil or a pharmaceutically acceptable salt thereof. In some embodiments, the compositions of the present disclosure comprise about 110 mg of sildenafil or a pharmaceutically acceptable salt thereof. In some embodiments, the compositions of the present disclosure comprise about 150 mg of sildenafil or a pharmaceutically acceptable salt thereof.

In some embodiments, the composition comprises:
about 2 wt. % to about 6 wt. % of sildenafil or a pharmaceutically acceptable salt thereof,
about 1 wt. % to about 5 wt. % of a bitterness reducing agent; and
about 6 wt. % to about 10 wt. % of a sweetener or flavoring agent.

In some embodiments, the composition comprises:
about 3 wt. % to about 5 wt. % of sildenafil or a pharmaceutically acceptable salt thereof,
about 2 wt. % to about 4 wt. % of a bitterness reducing agent; and
about 7 wt. % to about 9 wt. % of a sweetener or flavoring agent.

In some embodiments, the composition comprises:
about 4 wt. % of sildenafil or a pharmaceutically acceptable salt thereof,
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, silica, acacia, chocolate, marshmallow, vanilla and spearmint.

In some embodiments, the composition comprises:
about 3 wt. % to about 5 wt. % of sildenafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of silica,
about 0.25 wt % to about 0.75 wt. % of acacia,
about 1 wt % to about 4 wt. % of chocolate,
about 1 wt % to about 4 wt. % of marshmallow,
about 0.4 wt % to about 0.8 wt. % of vanilla and
about 0.5 wt % to about 1.5 wt. % of spearmint.

In some embodiments, the composition comprises:
about 4 wt. % of sildenafil or a pharmaceutically acceptable salt thereof,
about 3 wt. % NF01,
about 0.5 wt. % acesulfame,
about 0.5 wt. % steviol,
about 0.5 wt. % silica,
about 0.5 wt. % acacia,
about 3 wt. % chocolate,
about 3 wt. % marshmallow,
about 0.6 wt. % vanilla and
about 1 wt. % spearmint.

In some embodiments, the bitter active ingredient is tadalafil or a pharmaceutically acceptable salt thereof.

In some embodiments, the compositions comprise from about 5 mg to about 50 mg of tadalafil or a pharmaceutically acceptable salt thereof, including about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, and about 50 mg, including all ranges there between.

In some embodiments, the compositions comprise about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, and about 50 mg of tadalafil or a pharmaceutically acceptable salt thereof. In some embodiments, the compositions of the present disclosure comprise about 14 mg of tadalafil or a pharmaceutically acceptable salt thereof. In some embodiments, the compositions of the present disclosure comprise about 20 mg of tadalafil or a pharmaceutically acceptable salt thereof. In some embodiments, the compositions of the present disclosure comprise about 22 mg of tadalafil or a pharmaceutically acceptable salt thereof. In some embodiments, the compositions of the present disclosure comprise about 30 mg of tadalafil or a pharmaceutically acceptable salt thereof.

In some embodiments, the composition comprises:
about 2 wt. % to about 6 wt. % of tadalafil or a pharmaceutically acceptable salt thereof,
about 1 wt. % to about 5 wt. % of a bitterness reducing agent; and
about 6 wt. % to about 10 wt. % of a sweetener or flavoring agent.

In some embodiments, the composition comprises:
about 3 wt. % to about 5 wt. % of tadalafil or a pharmaceutically acceptable salt thereof,
about 2 wt. % to about 4 wt. % of a bitterness reducing agent; and
about 7 wt. % to about 9 wt. % of a sweetener or flavoring agent.

In some embodiments, the composition comprises:
about 4 wt. % of tadalafil or a pharmaceutically acceptable salt thereof,
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, silica, acacia, chocolate, marshmallow, vanilla and spearmint.

In some embodiments, the composition comprises:
about 3 wt. % to about 5 wt. % of tadalafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of silica,
about 0.25 wt % to about 0.75 wt. % of acacia,
about 1 wt % to about 4 wt. % of chocolate,
about 1 wt % to about 4 wt. % of marshmallow,
about 0.4 wt % to about 0.8 wt. % of vanilla and
about 0.5 wt % to about 1.5 wt. % of spearmint.

In some embodiments, the composition comprises:
about 4 wt. % of tadalafil or a pharmaceutically acceptable salt thereof,
about 3 wt. % NF01,
about 0.5 wt. % acesulfame,
about 0.5 wt. % steviol,
about 0.5 wt. % silica,
about 0.5 wt. % acacia,
about 3 wt. % chocolate,
about 3 wt. % marshmallow,
about 0.6 wt. % vanilla and
about 1 wt. % spearmint.

In some embodiments, the bitter active ingredient is a mixture of sildenafil or a pharmaceutically acceptable salt thereof and tadalafil or a pharmaceutically acceptable salt thereof.

In some embodiments, the compositions comprise about 5 mg to about 150 mg of sildenafil or a pharmaceutically acceptable salt thereof, including about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg and about 150 mg including all ranges there between and about 5 mg to about 50 mg of tadalafil or a pharmaceutically acceptable salt thereof, including about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, and about 50 mg, including all ranges there between.

In some embodiments, the compositions comprise about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg and about 150 mg of sildenafil or a pharmaceutically acceptable salt thereof and about 5 mg, about 6 mg, about 7 mg, about 8 mg, about 9 mg, about 10 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, and about 50 mg of tadalafil or a pharmaceutically acceptable salt thereof.

In some embodiments, the compositions comprise about 100 mg of sildenafil or a pharmaceutically acceptable salt thereof and about 20 mg of tadalafil or a pharmaceutically acceptable salt thereof. In some embodiments, the compositions comprise about 50 mg of sildenafil or a pharmaceutically acceptable salt thereof and about 5 mg of tadalafil or a pharmaceutically acceptable salt thereof. In some embodiments, the compositions comprise about 100 mg of sildenafil or a pharmaceutically acceptable salt thereof and about 20 mg of tadalafil or a pharmaceutically acceptable salt thereof. In some embodiments, the compositions comprise about 40 mg of sildenafil or a pharmaceutically acceptable salt thereof and about 14 mg of tadalafil or a pharmaceutically acceptable salt thereof. In some embodiments, the compositions comprise about 65 mg of sildenafil or a pharmaceutically acceptable salt thereof and about 22 mg of tadalafil or a pharmaceutically acceptable salt thereof. In some embodiments, the compositions comprise about 80 mg of sildenafil or a pharmaceutically acceptable salt thereof and 30 mg of tadalafil or a pharmaceutically acceptable salt thereof. In some embodiments, the compositions comprise about 110 mg of sildenafil or a pharmaceutically acceptable salt thereof and about 30 mg of tadalafil or a pharmaceutically acceptable salt thereof. In some embodiments, the compositions comprise about 150 mg of sildenafil or a pharmaceutically acceptable salt thereof and about 30 mg of tadalafil or a pharmaceutically acceptable salt thereof.

In some embodiments, the composition comprises:
about 0.2 wt. % to about 1.8 wt. % of sildenafil or a pharmaceutically acceptable salt thereof;
about 0.05 wt. % to about 0.35 wt. % of tadalafil or a pharmaceutically acceptable salt thereof;
about 1 wt. % to about 5 wt. % of a bitterness reducing agent; and
about 4 wt. % to about 8 wt. % of a sweetener or flavoring agent.

In some embodiments, the composition comprises:
about 0.5 wt. % to about 1.5 wt. % of sildenafil or a pharmaceutically acceptable salt thereof;
about 0.1 wt. % to about 0.3 wt. % of tadalafil or a pharmaceutically acceptable salt thereof;
about 2 wt. % to about 4 wt. % of a bitterness reducing agent; and
about 5 wt. % to about 7 wt. % of a sweetener or flavoring agent.

In some embodiments, the composition comprises:
about 1 wt. % of sildenafil or a pharmaceutically acceptable salt thereof,
about 0.2 wt. % of tadalafil or a pharmaceutically acceptable salt thereof
about 3 wt. % of a bitterness reducing agent; and
about 6 wt. % of a sweetener or flavoring agent.

In some embodiments, the bitterness reducing agent comprises NF01 and the sweetener or flavoring agent comprises acesulfame, steviol, spearmint, marshmallow, vanilla, chocolate, and acacia.

In some embodiments, the composition comprises:
about 0.5 wt. % to about 1.5 wt. % of sildenafil or a pharmaceutically acceptable salt thereof,
about 0.1 wt. % to about 0.3 wt. % of tadalafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.5 wt % to about 1.5 wt. % of spearmint,
about 1 wt % to about 3 wt. % of marshmallow,
about 0.1 wt % to about 0.5 wt. % of vanilla,
about 0.1 wt % to about 0.5 wt. % of chocolate, and
about 0.25 wt % to about 0.75 wt. % of acacia.

In some embodiments, the composition comprises:
about 1 wt. % of sildenafil or a pharmaceutically acceptable salt thereof,
about 0.2 wt. % of tadalafil or a pharmaceutically acceptable salt thereof,
about 3 wt. % of NF01,
about 0.5% of acesulfame,
about 0.5% of steviol,
about 1 wt. % of spearmint,
about 2 wt. % of marshmallow,
about 0.3 wt % of vanilla,
about 0.3 wt % of chocolate, and
about 0.5% of acacia.

In some embodiments, the bitter active ingredient comprises testosterone. In some embodiments, the compositions comprise about 0.5 mg to about 30 mg. In some embodiments, the compositions comprise about 2 mg of testosterone. In some embodiments, the compositions comprise about 5 mg of testosterone. In some embodiments, the compositions comprise about 10 mg of testosterone. In some embodiments, the compositions comprise about 12.5 mg of testosterone. In some embodiments, the compositions comprise about 20 mg of testosterone. In some embodiments, the compositions comprise about 30 mg of testosterone.

In some embodiments, the bitter active ingredient comprises Sildenafil, Tadalafil, and Testosterone. In some embodiments, the compositions comprise from about 40 mg to about 150 mg of Sildenafil, from about to 14 mg about 30 mg of Tadalafil, and from about 5 mg to about 30 mg of Testosterone. In some embodiments, the compositions comprise about 40 mg of Sildenafil, about 14 mg of Tadalafil, and about 5 mg of Testosterone. In some embodiments, the compositions comprise about 40 mg of Sildenafil, about 14 mg of Tadalafil, and about 10 mg of Testosterone. In some embodiments, the compositions comprise about 40 mg of Sildenafil, about 14 mg of Tadalafil, and about 12.5 mg of Testosterone. In some embodiments, the compositions comprise about 40 mg of Sildenafil, about 14 mg of Tadalafil, and about 20 mg of Testosterone. In some embodiments, the compositions comprise about 40 mg of Sildenafil, about 14 mg of Tadalafil, and about 30 mg of Testosterone. In some embodiments, the compositions comprise about 65 mg of Sildenafil, about 22 mg of Tadalafil, and about 5 mg of Testosterone. In some embodiments, the compositions comprise about 65 mg of Sildenafil, about 22 mg of Tadalafil, and about 10 mg of Testosterone. In some embodiments, the compositions comprise about 65 mg of Sildenafil, about 22 mg of Tadalafil, and about 12.5 mg of Testosterone. In some embodiments, the compositions comprise about 65 mg of Sildenafil, about 22 mg of Tadalafil, and about 20 mg of Testosterone. In some embodiments, the compositions comprise about 65 mg of Sildenafil, about 22 mg of Tadalafil, and about 30 mg of Testosterone. In some embodiments, the compositions comprise about 80 mg of Sildenafil, about 30 mg of Tadalafil, and about 30 mg of Testosterone. In some embodiments, the compositions comprise about 110 mg of Sildenafil, about 30 mg of Tadalafil, and about 30 mg of Testosterone. In some embodiments, the compositions comprise about 150 mg of Sildenafil, about 30 mg of Tadalafil, and about 30 mg of Testosterone.

In some embodiments, the bitter active ingredient comprises Sildenafil and Testosterone. In some embodiments, the compositions comprise from about 35 mg to about 45 mg of Sildenafil and from about 10 mg to about 30 mg of Testosterone. In some embodiments, the compositions comprise about 40 mg of Sildenafil and about 5 mg Testosterone. In some embodiments, the compositions comprise about 40 mg of Sildenafil and about 10 mg Testosterone. In some embodiments, the compositions comprise about 40 mg of Sildenafil and about 12.5 mg Testosterone. In some embodiments, the compositions comprise about 40 mg of Sildenafil and about 20 mg Testosterone. In some embodiments, the compositions comprise about 40 mg of Sildenafil and about 30 mg Testosterone.

In some embodiments, the bitter active ingredient comprises Tadalafil and Testosterone. In some embodiments, the compositions comprise from about 14 mg to about 30 mg of Tadalafil and from about 10 mg to about 30 mg of Testosterone. In some embodiments, the compositions comprise about 14 mg of Tadalafil and about 10 mg of Testosterone. In some embodiments, the compositions comprise about 14 mg of Tadalafil and about 12.5 mg of Testosterone. In some embodiments, the compositions comprise about 14 mg of Tadalafil and about 20 mg Testosterone. In some embodiments, the compositions comprise about 22 mg of Tadalafil and about 30 mg of Testosterone. In some embodiments, the compositions comprise about 30 mg of Tadalafil and about 30 mg of Testosterone.

In some embodiments, the bitter active ingredient comprises Sildenafil, oxytocin and Testosterone. In some embodiments, the compositions comprise from about 4 mg to about 10 mg of Sildenafil, from about 2 mg to about 8 mg of Testosterone and from about 20 IU to about 40 IU of Oxytocin. In some embodiments, the compositions comprise about 4 mg of Sildenafil, about 2 mg of Testosterone and about 20 IU of Oxytocin. In some embodiments, the compositions comprise about 8 mg of Sildenafil, about 4 mg of Testosterone and about 20 IU of Oxytocin. In some embodiments, the compositions comprise about 10 mg of Sildenafil, about 8 mg of Testosterone, and about 40 IU of Oxytocin.

In some embodiments, the present disclosure provides troches comprising one or more of sildenafil or a pharmaceutically acceptable salt, prodrug, or solvate thereof, tadalafil, papaverine, testosterone, or oxytocin as active ingredients. In some embodiments, the pharmaceutically acceptable salt of sildenafil comprises sildenafil citrate.

In some embodiments, the present disclosure provides a sublingual dosage form comprising one or more of sildenafil or a pharmaceutically acceptable salt, prodrug, or solvate thereof, sildenafil citrate, oxytocin, tadalafil, papaverine, testosterone bitterness reducing agent powder, acesulfame potassium powder, steviol glycosides 95% powder, spearmint oil, marshmallow liquid, vanilla flavor liquid, chocolate flavor liquid, acacia NF (spray dried gum Arabic), silica gel micronized powder, polyethylene glycol 1450 crystal, blue coloring (liquid), lemon flavoring, orange flavoring, flavorings, or colorants.

In some embodiments, the composition comprise

In some embodiments, troches for women are customized to work with the changes in the natural female hormones during sexual activity to enhance sensitivity and feelings of pleasure.

In some embodiments, the administration of the compositions of the present disclosure provide one or more of increased blood flow at a lower therapeutic dosage, greater sensitivity, longer lasting and harder erections, fast (e.g. 6 minutes) onset, fresh breath, spearmint flavor, combination of different APIs, effects lasting up to about 4 to about 6 hours, cost effectiveness, or discrete packaging.

In some embodiments, the present disclosure provides sublingual dosage forms comprising one or more of (Male) Sildenafil 10 mg to 110 mg Sublingual Troches, (Female) Sildenafil 5 mg to 40 mg Sublingual Troches with Oxytocin, (Male) Sildenafil/Tadalafil Combo 12.5 mg/2.5 mg Sublingual Troches, (Male) Sildenafil/Tadalafil Combo 40 mg/5 mg Sublingual Troches, (Male) Sildenafil/Tadalafil Combo 60 mg/10 mg Sublingual Troches, (Male) Sildenafil/Tadalafil Combo 80 mg/20 mg Sublingual Troches, (Male) Tadalafil Oral Suspension Orange/Lemon flavored Shot 15 ml, or Sildenafil/Papaverine/Testosterone/Oxytocin Combo Sublingual Troches.

In some embodiments, the bitter active ingredient is selected from the group consisting of Quinidine, Quinine, humulon, Quinidine, benzaldehyde, Magnesium Sulfate, Lupulon, Lupulone, fennel oil, Humulon, Humulone, Benzoin, Quinine, Quinine, Quinine bimuriate, Quinine dihydrochloride, Quinine, Quinine hydrochloride, Absinthin, Arborescin, Arglabin, Aristolochic acid, Artemorin, Caffeine, Cascarillin, Coumarin, Cucurbitacin B, Falcarindiol, Noscapine, Papaverine, Parthenolide, Quassin, Azathioprine, Benzamide, Carisoprodol, Chlorhexidine, Chlorpheniramine, Diphenhydramine, diphenidol, diphenylthiourea, sulfocarbanilide, sym-Diphenylthiourea, thiocarbanilide, Divinyl sulfoxide, Flufenamic Acid, Haloperidol, Sodium Benzoate, Thiamine, chloramphenicol, yohimbine, amarogentin, Adhumulone, Cohumulone, Colupulone, Isoxanthohumol, Xanthohumol, dextromethorphan, sodium thiocyanate, trans-isohumulone, trans-isocohumulone, Adlupulone, cis-isocohumulone, cis-isohumulone, cis-isoadhumulone, trans-isoadhumulone, sodium cyclamate, chloroquine, 8-prenylnaringenin, Picrotoxinin, (−)-alpha Thujone, brucine, colchicine, dapsone, denatonium benzoate, 1,10-phenanthroline, cromolyn, cucurbitacin E, Cucurbitacins, cycloheximid, Cycloheximide, erythromycin, strychnine, famotidine, Amygdalin, D, Arbutin, Helicin, sinigrin, acetylthiourea, allyl isothiocyanate, Caprolactam, dimethylthioformamide, Ethylpyrazine, N-ethylthiourea, Ethylene Thiourea,N,N-ethylene thiourea, limonin, methimazole, N-methylthiourea, phenethyl isothiocyanate, Phenylthiocarbamide (PTC), Propylthiouracil, Acetaminophen, acesulfame K, aloin, Grosheimin, grossheimin, saccharin, andrographolide, cnicin, crispolide, Hydrocortisone, orphenadrine, tatridin B, methadone, Cinnamedrine, Citric Acid Ethyl ester, Triethyl citrate, Clobutinol, Clonixin, Cocaine Hydrochloride, Alverine, Codamine, Colubrines, Columbin, Conessine, Coriamyrtin, Coumarilic Acid, Cyclexedrine Hydrochloride, Cyclobarbital, Cyclobarbital, Cyclopentamine Hydrochloride, Deferiprone, 2-Amino-5-nitrothiazole, Dehydrocholic Acid, Dextroamphetamine Sulfate, Dextromoramide Bitartrate, Dibekacin Sulfate, Dicumarol, Dicyclomine Hydrochloride, 3,5-Diiodosalicylic Acid, Diltiazem Hydrochloride, Dimemorfan Phosphate, Dimethocaine Hydrochloride, Dimethyl Sulfoxide, 3,4-Dinitrobenzoic Acid, Dipyridamole, Aminophylline, &gamma;,&gamma;′ -Dipyridyl Dihydrate,gamma,gamma′-Dipyridyl, Domiphen Bromide, Drotebanol, Dyphylline, Edrophonium Bromide, Epirizole, Eprazinone Dihydrochloride, Ethyl Biscoumacetate, Etilefrin Hydrochloride, Fencamine Hydrochloride, Fenproporex Hydrochloride, Fentanyl Citrate, Fluorotoluene, Fraxin, Furfuryl Alcohol, Ammonium Picrate, Galegine, Gamabufotalin, Gentiobiose, Ginkgolide C, Ginkgolide A, Ginkgolide B, &#946;-Glucogallin, Glucovanillin, Glutaronitrile, Amobarbital, Amobarbital Sodium salt, Guaifenesin, Harman, Helenalin, Heptabarbital, Hexobarbital Sodium salt, Homidium Bromide, Amodiaquin Dihydrochloride dihydrate, Hydrogen Peroxide, Hydroxychloroquine sulfate, Hydroxyzine Dihydrochloride, Imipramine <i>N-</i>Oxide Hydrochloride, Improsulfan Tosylate, Acecarbromal, Amphetamine Sulfate, Amphetamine Phosphate, Inaperisone Hydrochloride, Iodinated Glycerol, Ipodate Sodium salt, L-Isoleucine, Isometheptene Mucate, Isopropyl Alcohol, Isoxsuprine, Khellin, Lafutidine, Lappaconitine, Linamarin, Lincomycin Hydrochloride monohydrate, Lithium Bromide, Amylocaine Hydrochloride, Lycoctonine, Lycopodine, Magnesium Bromide, Magnesium Chlorate Hexahydrate, Magnesium Lactate Trihydrate, Magnesium Sulfate Heptahydrate, Meconin, <i>1-</i>Menthone, Meperidine Hydrochloride, Mephenesin, Meprobamate, Mersalyl, Methacycline Hydrochloride, Methadone Hydrochloride, Methamphetamine Hydrochloride, Methantheline Bromide, Methoxsalen, xanthotoxin, Methoxyphenamine Hydrochloride, Methylbenzethonium Chloride Monohydrate, 5,5′ Methylenedisalicylic Acid, Methylergonovine Maleate, 6-Methyl-2-thiouracil,Methylthiouracil, Methyprylon, Anethole Trithione, Metralindole Hydrochloride, Mibefradil Dihydrochloride, Morphine Hydrochloride, Moveltipril, calcium salt, Naphazoline Hydrochloride, Neoquassin, Neostigmine Bromide, Nialamide, Nikethamide, Nitrofurazone, Nomilin, Norleucine D(−)-Form, Oleuropein, Antazoline Phosphate, Antazoline Hydrochloride, Oxeladin, Oxyacanthine, 4,4′-Oxydi-2-butanol, Pancuronium Bromide, Pantothenic Acid Calcium salt, Antipyrine, Pentamidine Isethionate, 1,5-Pentanediol, Pentobarbital Sodium salt, Pentoxifylline, Pentylenetetrazole, Periplocymarin, Phenacetin, Phenazopyridine Hydrochloride, <i>p</i>-Acetanisidine, ANTU, Phenobarbital, Phenprobamate, Phentolamine, Phenyl Biguanide, Phenylephrine, Phenytoin, Phloridzin, Pholcodine, Diethyl phthalate, Phthalic Acid Ethyl ester, Phthalylsulfathiazole, Picoperine Hydrochloride, Picric Acid, Picrocrocin, saffron-bitter, Picromycin, pikromycin, albomycetin, amaromycin, Picrotoxin, Pifarnine, Pifazin, Pipemidic Acid, Piperidione, Pipradrol Hydrochloride, Plaunotol, Potassium Guaiacolsulfonate, Potassium Sulfate, Procaine Dihydrate, Propafenone Hydrochloride, Propallylonal, Aprobarbital Sodium salt, Aprobarbital, Propamidine Isethionate, Propoxyphene, Propoxyphene, Propyphenazone, Pseudoephedrine, Psychotrine Tetrahydrate, Pyrazole, Pyronaridine Tetraphosphate, Quebrachamine, Quercetin, Quinacrine Dihydrochloride dihydrate, Quinazoline, Quinovic Acid, Arecoline Hydrobromide, Retamine, Rhododendrin, D-salicin, Salicin, Artemisin, Salicylamide, &#945;-Santonin (−)-Form, Scillaren, Secobarbital Sodium, Senecionine, Sodium Bromide, Sodium Phenolsulfonate Dihydrate, Streptomycin, Strontium Bromide, Succinylcholine Chloride, Sucrose Octaacetate, Sulfamerazine Monosodium salt, Sulfameter, Sulfamethoxazole, Sulfamethoxypyridazine, Sulfisoxazole, Sulfobromophthalein Sodium, Sulfonethylmethane, Suxibuzone, Swertiamarin, Tacrine Hydrochloride, Talbutal, Terpin cis-Form hydrate, Terpin hydrate, terpinol, Tetracaine Hydrochloride, Aconine, Atranorin, Tetraethylammonium Hydroxide, Theophylline, Theophylline Sodium acetate, Thiacetazone, Thiamine Hydrochloride, Vitamin b1, Thiosinamine, 2-Thiouracil, Tiaramide Hydrochloride, Tiliacorine, Tinoridine Hydrochloride, Tolazoline Hydrochloride, Topiramate, Tramadol Hydrochloride, Trapidil, Triacetin, Glycerol tributyrate, Tributyrin, Tridihexethyl Iodide, Trimethadione, Trimethaphan Camsylate, Azintamide, Trimethoprim, Ursodiol, Valpromide, Veatchine, Verbenalin, Viquidil Hydrochloride, Warfarin Sodium salt, Carnosol, Ganoderic Acid A, Amprotropine Phosphate, Cetraric Acid, Chonemorphine, Cycrimine Hydrochloride, Diperodon Hydrochloride, Dodecarbonium Chloride, Ethylbenzhydramine Hydrochloride, Ethylhydrocupreine, Glaucarubin, Melilotoside, Azacyclonol Hydrochloride, Butallylonal, Butallylonal Sodium salt, Camphotamide, Chlorbetamide, Chlorhexadol, Chrysamminic Acid, Bakankosin, Cyclopentobarbital, Diethylbromoacetamide, Dioxypyramidon, Garryine, Hexethal Sodium, Homocamfin, <i>p-</i>Lactophenetide, Narcobarbital, Nealbarbital, Pentamethonium Bromide, Phenallymal, Phenylmethylbarbituric Acid, Vinbarbital Sodium, Chlormetacrine Dihydrochloride, Barbital, Barbital Sodium salt, Barium Chloride Dihydrate, Benzethonium Chloride, Benzilic Acid, Bretylium Tosylate, Bufotoxin, Bupropion Hydrochloride, Butabarbital Sodium salt, Butalbital, Butethal, Butethamine meta-Isomer hydrochloride, Allobarbital, Calcium Hydroxide, Calcium Phenolsulfonate, Carbimazole, Carbinoxamine Maleate, Cefazolin Sodium salt, cefpodoxime 1-(Isopropoxycarbonyloxy)ethyl ester, cefpodoxime proxetil, Cephaeline, Chenodiol, Chloral Hydrate, Chlorbenzoxamine Dihydrochloride, Chlormerodrin, Chloroprocaine, chloroquine phosphate, Cinchophen, Naringenin, Phenyl beta-D-glucopyranoside, Promethazine, nicotine, ZINC01532585, Emetine, Naringin, Cynaropicrin, Gallic acid, Laevuflex, Meranzin, Acetosulfam, hydroxylupanine, Prunin, EINECS 227-420-9, piperidine, Gentianine, gatifloxacin, Glyceryl caprate, Umbelliferones, alpha-Linolenic Acid, ofloxacin, procainamid, pirenzapin, Ciprofloxacin, Enoxacin, Lomefloxacin, Omeprazole, Dexamethasone, Prednisone, 1-nitronaphthalene, Ethyl Benzoate, Picoline, Diisobutylamine, Cyclooctanone, Clindamycin, Acetylpyrazine, Labetalol-HCl, Clarithromycin, Cinchonine, Lupinine, Doxepin, Thioacetanilide, Goitrin, Enalapril, 1,1-Dimethylbiguanide, Spartein Sulfate Pentahydrate, 2,6-Dimethylpiperidine, 1-methy-2-quinolinone, Atropine Sulfate, ,Alerlisin, Virlix, Zyrtec, L-histidine, Isobutyl salicylate, Isoamyl salicylate, Eugenyl methyl ether, Ethyl benzoylacetate, Acetophenone, Acetanisole, A,a-dimethylphenethyl alcohol, Ethyl phenylacetate, Isoamyl propionate, 3,7-dimethyl-1-octanol, 1,3-butylene glycol, Ethyl formate, Isobutyl acetate, Glyceryl tripropanoate, Benzyl acetate, Citronellyl propionate, D-citronellol, Hexyl acetate, P-dimethoxybenzene, Isobutyl propionate, 2-heptanol, 3-heptanol, Isopropyl propionate, D-fenchone, Fenchyl alcohol, Butyl levulinate, Ethyl maltol, Isoamyl acetate, Glucose pentaacetate, D-camphor, Isoeugenyl methyl ether, Citral (neral), Geranyl acetate, Geranyl formate, Cinnamyl alcohol, Cinnamyl formate, Geranyl propionate, Phenylacetaldehyde, Taurine, Propyl gallate, Theobromine, Phenethyl alcohol, L-leucine, L-phenylalanine, Methyl acetate, 6-methylcoumarin, 2-methoxy-4-methylphenol, P-mentha-1,4-diene, 1-isopropyl-4-methyl, Phenethyl isobutyrate, 3-phenylpropyl isobutyrate, Phenethyl formate, Propyl propionate, Propyl acetate, Propyl formate, 2-octanone, Piperonal, Methyl anthranilate, O-methoxybenzaldehyde, Phenethyl isovalerate, Rhodinyl acetate, Nonyl alcohol, Nonyl acetate, Piperonyl acetate, 6-methyl-5-hepten-2-one, Propyl isovalerate, 1-penten-3-ol, Terpinyl formate, Menthyl isovalerate, 3-phenylpropyl acetate, P-methoxybenzaldehyde, Phenylacetaldehyde dimethyl acetal, Linalyl formate, Rhodinyl formate, A-terpinyl anthranilate, Terpinyl propionate, A-methylbenzyl acetate, Polysorbate 20, Terpinyl isovalerate, Nerol, Polysorbate 80, 2-tridecenal, Neryl isovalerate, Propyl 2-furanacrylate, 3-methyl-2-phenylbutyraldehyde, Rhodinyl isovalerate, Polysorbate 60, Santalyl acetate, a- and b-, Genistein, Daidzein, (+/−) Equol, Prunetin?□, Potassium chloride, Alpha-tetralone, Berberine, Prednisolone, Epigallocatechin gallate, egcg, Catechin, Tangeretin, Nobiletin, Sinensetin, (−)-Epicatechin, EC, Epicatechin, (−)-epigallocatechin (EGC), epigallocatechin, Gallocatechol?□, (−)-Epicatechin gallate, ECg, Epicatechin gallate, Genistin, Daidzin, Sinigrin?□, Progoitrin, Glucobrassicin, Hydrogen cyanide, Sulfur dioxide, Alitame-11 isomer, Coumestrol, Vancomycin, L-trp, L-tryptophan, Leu-trp, Phe-trp,Phe-trp phenylalanyltryptophan, Trp-leu, Trp-phe, Trp-trp, Leu-leu-leu, Leucyl-leucyl-leucine, Gly-leu,Glycyl-1-leucine,Glycylleucine, gly-phe, glycyl-1-phenylalanine, Glycylphenylalanine, Phe-leu, Phenylalanylleucine, Pro-arg, Prolylarginine, Phe-phe-phe, Phenylalanyl-phenylalanyl-phenylalanine, 6-nitrosaccharin, 7-nitrosaccharin, 5 nitrosaccharin, apigenin, chrysin, chrysoeriol, 5,2′-dihydroxyflavone, 5,4′-dihydroxyflavone, 6,4′-dihydroxyflavone, 7,4-′dihydroxyflavone, 5,7′-dimethoxyflavonee, 6,7′-dimethoxyflavone, flavone, genkwanin, 5-hydroxyflavone, 4′-hydroxyflavone, 4′-hydroxy-6-methoxyflavone, 4′-hydroxy-7-methoxyflavone, luteolin, 6-methoxyluteolinb, scutellarein, tricetin, 5,7,2′-trihydroxyflavone, 7,3′,4′-trihydroxyflavone, 5,7′,4′-trimethoxyflavone, datiscetinb, fisetin, gossypetind, herbacetin, isorhamnetin, kaempferol, Kaempherol, morin, myricetind, quercetagetin, 3,6,3,4-tetrahydroxyflavone, 3,7,4-trihydroxyflavone, eriodictyolb, flavanone, Hesperetin, hesperitin, homoeriodictyolc, liquiritigenin, pinocembrin, fustin, silibinind, (+)-taxifolin, taxifoline, butein, chalcone, 3,2-dihydroxychalcone, eriodictyolchalconec, 4-hydroxychalcone, isoliquiritigenin, 2,2,4-trihydroxychalcone, 4,2,5-trihydroxychalconee, phloretinb, cyanidin chloridec, pelargoninidin chloride, acetylgenistin, biochanin Ag, 7,4-dimethoxyisoflavone, formomonetin, glyciteing, glyciting, 7-hydroxyisoflavoneg, isoflavone, 6,7,4-trihydroxyisoflavone, 7,8,4-trihydroxyisoflavone, 7,3,4-trihydroxyisoflavone, resveratrol, sulfuretinc, xanthonee, 4-hydroxyanisol, Hg-12, Aglycon H.g.-12, 1-Naphthoic acid, 1,8-naphthaladehyde acid, Picrotin, Piperonylic acid, Herbolide D, Pantoprazole, Glimepiride, Malathion, Mefenamic acid, Pemirolast, Miconazole, Salsalate, Niflumic acid, Diclofenac, Artecalin, 1&alpha,6&alpha,8&alpha-trihydroxy-5&alpha,7&betaH-guaia-3,9,11(13)-trien-12-oic acid, costunolide, denatonium saccharide, Docosahexaenoic Acid, homoserine lactone, N-hexanoyl-L-, homoserine lactone, N-(3-oxooctanoyl)-L-, homoserine lactone, N-(3-oxohexanoyl)-L-, homoserine lactone, N-butyryl-L-, Progesterone, Salicylic acid, sparteine, Sucralose, 2-Heptyl-3-hydroxy-quinolone, 5-propyl-2-thiouracil, beta carotene, Androsterone, atropine, Berberine chloride, cis-11-Methyl-2-dodecenoic acid, creatinin, creatinine, Cucurbitacin D, Cucurbitacin I, lidocaine, ouabain, pantothenic acid, pyrocatechin, phenylbutazone, phenylethyl isothiocyanate, raffinose undecaacetate, tatridin A, TAUROCHOLIC ACID, avenanthramide 2c, avenanthramide 2p, avenanthramide 2f, avenanthramide 1p, avenanthramide 1c, avenanthramide 1f, avenanthramide 1s, avenanthramide 2s, avenacoside-A, avenacoside-B, avocadyne, avocadene, 1-acetoxy-2,4-dihydroxyheptadeca-16-ene, 1-acetoxy-2,4-dihydroxyheptadeca-16-yne, 1-acetoxy-2-hydroxy-4-oxoheptadeca-16-ene, 1-acetoxy-2-hydroxy-4-oxoheptadecane, 1-acetoxy-2-hydroxy-4-oxo-octadeca-12-ene, 1-acetoxy-2-hydroxy-4-oxoheneicosa-5,12,15-triene, 1-acetoxy-2,4-dihydroxyheneicosa-12,15 -diene, persin, Glyceryl monolinoleate, Glyceryl Monostearate, 1-O-palmitoyl-glycerol, 1-O-oleoyl-glycerol, 2-O-oleoyl-glycerol, 6-methoxymellein, eugenin, gazarin, falcarinol, falcarindiol 3-acetate, 4′-hydroxytunicatachalcone, isoxantholupon, 1′,2′-dihydroxanthohumol C, 1′,2′-dihydroisoxanthohumol C, 1′,2′-dihydroxanthohumol K, xanthohumol P, isoxanthohumol P, 5′-prenylxanthohumol, 1′,2′-dihydroxanthohumol F, xanthohumol D, xanthohumol B, xanthohumol C, xanthohumol H, isoxanthohumol H, xanthohumol N, 2′-hydroxy-xanthohumol M, xanthohumol I, xanthohumol O, xanthohumol L, xanthohumol M, isoxanthohumol M, 2′,3′-dehydrocyclohumulohydrochinon, 1-methoxy-4-prenylphloroglucinol, cis-p-coumaric acid methyl ester, trans-p-coumaric acid methyl ester, cis-p-coumaric acid ethyl ester, p-coumaric acid ethyl ester, trans-p-coumaric acid ethyl ester, quercetin-3-O-?-D-glucopyranoside, kaempferol-3 -O-?-D-glucopyranoside, kaempferol-3-O-?-D-(6?-malonyl)glucopyranoside, 1-O-beta-D-(2-methylpropanoyl)phloroglucinol glucopyranoside, 1-O-beta-D-(2-methylbutyryl)phloroglucinol glucopyranoside, phloroisovalerophenon-3,5-di-C-beta-D-glucopyranoside, trans-N-feruloyltyramine, nortricycloadlupone, 2?,3?-epoxyxanthohumol, xanthohumol G, cohumulinone, humulinone, adhumulinone, cis-cohumulinic acid, cis-humulinic acid, cis-adhumulinic acid, trans-cohumulinic acid, trans-humulinic acid, trans-adhumulinic acid, cis-alloisocohumulone, cis-alloisohumulone, cis-alloisoadhumulone, trans-alloisocohumulone, trans-alloisohumulone, trans-alloisoadhumulone, hydroxy-cis-alloisocohumulone, hydroxy-cis-alloisohumulone, hydroxy-cisalloisoadhumulone, hydroxy-trans-alloisocohumulone, hydroxy-trans-alloisohumulone, hydroxy-trans-alloisoadhumulone, hydroperoxy-cis-alloisocohumulone, hydroperoxy-cis-alloisohumulone, hydroperoxy-cis-alloisoadhumulone, hydroperoxy-trans-alloisocohumulone, hydroperoxy-trans-alloisohumulone, hydroperoxy-trans-alloisoadhumulone, tricyclocohumol, tricyclohumol, tricycloadhumol, tricyclocohumene, tricyclohumene, tricycloadhumene, tricyclocohumulactol, tricyclohumulactol, tricycloadhumulactol, scorpiocohumol, scorpiohumol, cohulupone, hulupone, adhulupone, hulupinic acid, tetracyclocohumol, tetracyclohumol, tetracycloadhumol, hydroxytricyclocolupone, hydroxytricyclolupone, hydroxytricycloadlupone, hydroperoxytricyclocolupone, hydroperoxytricyclolupone, hydroperoxytricycloadlupone, tricyclocolupone, tricyclolupone, tricycloadlupone, dehydrotricyclocolupone, dehydrotricyclolupone, dehydrotricycloadlupone, nortricyclocolupone, nortricyclolupone, furostanol saponin (TFI), Officinalisnin I, Officinalisnin II, quinizolate, (2S,6S)-octahydrodipyrrolo[1,2-a;1?,2?-d]pyrazine-5,10-dione, cis-cyclo(L-Pro-L-Pro), pyrrolidinohexose reductone, 7-methyl-2,3,6,7-tetrahydrocyclopenta[b]azepin-8(1H)-one, 2,3 -Di(pyrrolidine-1-yl)-5-hydroxy-5-methyl-2-cyclopentene-1-one,bis(pyrrolidino)hexose reductone, 3-O-[?-L-Rhap-(1?2)-{?-L-rhap-(1?4)}-?-D-glcp]-26-O-[?-D-glcp]-(25R)-22-hydroxyfurost-5-ene-3?,26-diole, 3-O-[?-L-Rhap-(1?2)-{?-L-rhap-(1?4)}-?-D-glcp]-26-O-[?-D-glcp]-(25S)-22-hydroxyfurost-5-ene-3?,26-diole, (25R)-Furost-5-ene-3?,22,26-triol-3-O-[?-L-rhamnopyranosyl-(1?4)-?-D-gluco-pyranosid]-26-O-?-D-glucopyranoside, (25S)-Furost-5-ene-3?,22,26-triol-3-O-[?-L-rhamnopyranosyl-(1?4)-?-D-gluco-pyranosid]-26-O-?-D-glucopyranoside, (25R)-Furostane-3?,22,26-triol-3-O-[?-L-rhamnopyranosyl-(1?4)-?-D-glucopyranoside]-26-O-?-D-glucopyranoside, (25S)-Furostane-3?,22,26-triol-3-O-[?-L-rhamnopyranosyl-(1?4)-?-D-glucopyranoside]-26-O-?-D-glucopyranoside, 3-O-[?-L-Rhamnosypyranosyl-(1?2)-?-L-rhamnosypyranosyl-(1?4)-?-D-glucopyranosyl]-(25S)-spirost-5-en-3?-ole, 3-O-[?-D-Glucopyranosyl-(1?2)-?-D-xylopyranosyl-(1?4)-?-Dglucopyranosyl]-(25S)-5?-spirostan-3?-ole, cis-cyclo(Gly-L-Pro), cis-cyclo(L-Phe-L-Pro), cis-cyclo(L-Leu-L-Pro), cis-cyclo(Gly-L-Phe), cis-cyclo(L-Asp-L-Phe), cis-cyclo(L-Leu-Gly), cis-cyclo(L-Ala-L-Leu), cis-cyclo(L-Ala-L-Phe), cis-cyclo(L-Ala-L-Pro), cis-cyclo(L-Ala-L-Val), cis-cyclo(L-Asn-L-Phe), cis-cyclo(L-Val-L-Phe), cis-cyclo(L-Pro-L-Thr), cis-cyclo(L-Pro-L-Tyr), cis-cyclo(L-Leu-L-Phe), cis-cyclo(L-Val-L-Pro), cis-cyclo(L-Phe-L-Ser), cis-cyclo(L-Val-L-Val), cis-cyclo(L-Ala-L-IIe), cis-cyclo(L-Ala-L-Tyr), cis-cyclo(L-Val-L-Leu), cis-cyclo(L-Val-L-Tyr), cis-cyclo(L-IIe-L-Phe), cis-cyclo(L-IIe-L-Pro), trans-cyclo(L-Val-D-Phe), cis-cyclo(L-Ala-Gly), trans-cyclo(D-Ala-L-Pro), trans-cyclo(D-Ala-L-Val), creatine, L-TYROSINE, L-lysine, L-valine, L-arginine, Xanthine, hypoxanthine, inosine, adenosine, homoquinizolate, procyanidin C1, gallic acid ethyl ester, vanillic acid ethyl ester, syringic acid ethyl ester, Ethyl trans-caffeate, caffeic acid ethyl ester, ethyl 4-hydroxy-3-methoxycinnamate, ferulic acid ethyl ester, ferulic acid ethyl ester, Gamma-Glu-Tyr, alpha-Glu-Tyr, alpha-Glu-Trp, sodium sulfate, neohesperidine, propanolol, L-menthol, capsaicin, urea, aspirin, CaCl2,calcium chloride, magnesium chloride, phenylpropanolamine HCl, diosbulbin D, (+)-Quercoresinoside A, (−)-Quercoresinoside B, (−)-3-Methoxy-4-hydroxyphenol 1-O-?-D-(6?-O-galloyl)-glucopyranoside, per-oxy artemorin, epizaluzannin C, germacradien-6,11-dihydroxy-8,12-olide, herbolide A, herbolide D acetate, nobilin, parthenin, santamarine, sintenin, speciformin acetate, tatridn A acetate, dihydro taurin, umbellifolide, vulgarolide, zaluzannin D, marrubiin, teuflavin, teuflavoside, teumarin, strychnine-N-oxide, Convallatoxin, 3-o-(p-D-glucopyranosyl)-oleanolicacid, 3-O-[(? -D-xylopyranosylx 1?3)]-[?-D-glucuronopyranosyl]-oleanolicacid, 3-O-[(?-D-xylopyranosylx 1?3)]-[?-D-glucuronopyranosyl]-oleanolic acid-[?-D-glucopyranosyl]ester, 2-{4-[(E)-(Hydroxyimino)methyl]-3-cyclohexen-1-yl}-1-methoxy-2-propanol, 2-{4-[(E)-(Hydroxyimino)methyl]-3-cyclohexen-1-yl}-2-methoxy-1-propanol, 2-{4-[(E)-(Hydroxyimino)methyl]-3-cyclohexen-1-yl}-1,2-propanediol, (E)-1-(3,6-Dihydro-2H-pyran-4-yl)-N-hydroxymethanimine, (E)-1-(5,6-Dihydro-2H-pyran-3-yl)-N-hydroxymethanimine, (E)-1-(3,6-Dihydro-2H-thiopyran-4-yl)-N-hydroxymethanimine, (E)-1-[4-(1,2-Dimethoxy-2-propanyl)-1-cyclohexen-1-yl]-N-hydroxymethanimine, (E)-N-Hydroxy-1-[5-(1-methoxyethyl)-1-cyclohexen-1-yl]methanimine, (E)-N-Hydroxy-1-[5-(methoxymethyl)-1-cyclohexen-1-yl]methanimine, (E)-N-Hydroxy-1-[5-(1-methoxyethyl)-1,4-cyclohexadien-1-yl]methanimine, (E)-1-(Bicyclo[2.2.2]octa-2,5-dien-2-yl)-N-hydroxymethanimine, bergapten, isopimpinellin, skimmianine, 2-(1H-indol-3-yl)ethanol, QLFNPSTNPWHSP, QLFGPNVNPWHNP, QLFNPSTNPW, QLFNPSTNPW-COOC2H5, QLFGPNVNPW-COOC2H5, stevioside, Rebaudioside A, Rebaudioside B, Rebaudioside C, Rebaudioside D, steviolbioside, dulcoside A, rubusoside, Lidocaine HCl, Donepezil, DIAZEPAM, S praziquantel, PGG, procyanidin B3, procyanidin C2, malvidin-3-glucoside, cyanidin-3-glucoside, Pyridostigmine Bromide, Trp-Pro, Trp-Trp-Trp, trans-pseudoisoeugenol 2-methyl butyrate, Calcium lactate, procyanidin B1, procyanidin B2, protocatechuic acid ethyl ester, N1,N4,N8-Tris(dihydrocaffeoyl)spermidine, N1,N8-Bis(dihydrocaffeoyl)spermidine, 1-O-Sinapoyl ?-D-glucoside, 5-O-Caffeoylquinic acid, rabdosianone I, rabdosianone II, lactucin, lactucopicrin, lactucin-15-oxalate, lactucopicrin-15-oxalate, 8-deoxylactucin-15-sulphate, Acortatarin A, Pollenopyrroside A, epi-Acortatarin A, Xylapyrroside A, 5-Hydroxymethyl-1-[(5-hydroxymethyl-2-furanyl)methyl]-1Hpyrrole-2-carbaldehyde, (2R)-3-(Allylthio)-2-[(4R)-4-(allylthiomethyl)-6-formyl-3-oxo-3,4-dihydropyrrolo-[1,2-a]pyrazin-2(1H)-yl] Propanoic Acid, NULL, ranitidine hydrochloride, geshoidin, alpha Solanine, solanine, Matrine, esculine Aesculin, 2-Naphthyl beta-D-glucopyranoside, Methyl beta-D-glucoside, Phenyl beta-D-galactopyranoside, phenyl alpha-D-glucopyranoside, 2-nitro phenyl beta-D-glucopyranoside, Obacunone, momordicine, lactucopicrin, 11?,13 dihydrolactucin, Dehydrocyanaropicrin, Gentiopicrin, imidazole, Pyridazine, 1,3,7,9-Tetramethyluric acid, Denatonium Chloride, choline chloride, sinapic acid, Poncirin, Scabraside, 5-Methyl-2,3-dipyrrolidin-1-ylcyclopent-2-en-1-one, Pinellic acid; 9,12,13-TriHOME, Artabsin, digitoxin, alpha chaconine, tomatine, solanidine, isolupinine, and caffeic acid.

In some embodiments, the bitter active ingredient is selected from the group consisting of 1-Phenylcyclohexylamine, 1-Piperidinocyclohexanecarbonitrile, 4-Anilino-N-phenethyl-4-piperidine (ANPP), Alfentanil CI, Amobarbital, Amphetamine, Anileridine, Bezitramide, Carfentanil, Coca Leaves, Cocaine, Codeine, Dextropropoxyphene, Dihydrocodeine, Dihydroetorphine, Diphenoxylate, Dronabinol CII, Ecgonine, Ethylmorphine, Etorphine HCl, Fentanyl, Glutethimide, Hydrocodone, Hydromorphone, Isomethadone, Levo-alphacetylmethadol, Levomethorphan, Levorphanol, Lisdexamfetamine, Meperidine, Meperidine intermediate-A, Meperidine intermediate-B, Meperidine intermediate-C, Metazocine, Methadone, Methadone intermediate, Methamphetamine, Methylphenidate, Metopon, Moramide-intermediate, Morphine, Nabilone, Noroxymorphone, Opium extracts, Opium fluid extract, Opium poppy, Opium tincture, Opium (granulated), Opium (powdered), Opium (raw), Oripavine, Oxycodone, Oxymorphone, Pentobarbital, Phenazocine, Phencyclidine, Phenmetrazine, Phenylacetone, Piminodine, Poppy Straw, Poppy Straw Concentrate, Racemethorphan, Racemorphan, Remifentanil, Secobarbital, Sufentanil, Tapentadol, Thebaine, Thiafentanil, [3,2-c]-furazan-5α-androstan-17β-ol, [3,2-c]pyrazole-androst-4-en-17β-ol, 13Beta-ethyl-17beta-hydroxygon-4-en-3-one, 17Alpha-methyl-3alpha,17beta-dihydroxy-5alpha-androstane, 17Alpha-methyl-3beta, 17beta-dihydroxy-5alpha-androstane, 17Alpha-methyl-3beta, 17beta-dihydroxyandrost-4-ene, 17Alpha-methyl-4-hydroxynandrolone (17alpha-methyl-4-hydroxy-17beta-hydroxyestr-4-en-3-one), 17Alpha-methyl-delta1-dihydrotestosterone (17beta-17-Alpha-methyl-1-testosterone, hydroxy-17alpha-methyl-5alpha-androst-1-en-3-one), 17α-Methyl-5α-androstan-17β-ol, 17α-methyl-androst-2-ene-3,17β-diol, 17α-methyl-androsta-1,4-diene-3,17β-diol, 17α-Methyl-androstan-3-hydroxyimine-17β-ol, 17β-Hydroxy-androstano[2,3-d]isoxazole, 17β-Hydroxy-androstano[3,2-c]isoxazole, 18a-Homo-3-hydroxy-estra-2,5(10)-dien-17-one, 19-Nor-4,9(10)-androstadienedione, 19-Nor-4-androstenediol (3beta,17beta-dihydroxyestr-4-ene; 3 alpha, 17beta-dihydroxyestr-4-ene), 19-Nor-4-androstenedione (estr-4-en-3,17-dione), 19-Nor-5-androstenediol (3beta,17beta-dihydroxyestr-5-ene; 3alpha,17beta-dihydroxyestr-5-ene), 19-Nor-5-androstenedione (estr-5-en-3,17-dione), 1-Androstenediol (3beta,17beta-dihydroxy-5alpha-androst-1-ene; 3alpha,17beta-dihydroxy-5alphaandrost-1-ene), 1-Androstenedione (5alpha-androst-1-en-3,17-dione), 2α,17α-dimethyl-17β-hydroxy-5β-androstan-3-one, 2α,3α-epithio-17α-methyl-5α-androstan-17β-ol, 3Alpha,17beta-dihydroxy-5alpha-androstane, 3Beta,17beta-dihydroxy-5alpha-androstane, 3β-hydroxy-estra-4,9,11-trien-17-one, 4-Androstenediol (3beta,17beta-dihydroxy-androst-4-4-ADene), 4-Androstenedione (androst-4-en-3,17-dione), 4-chloro-17α-methyl-17β-hydroxy-androst-4-en-3-one, 4-chloro-17α-methyl-17β-hydroxy-androst-4-ene-3,11-dione, 4-chloro-17α-methyl-androst-4-ene-3β,17β-diol, 4-chloro-17α-methyl-androsta-1,4-diene-3,17β-diol, 4-Dihydrotestosterone (17beta-hydroxyandrostan-3-one), 4-Hydroxy-19-nortestosterone (4,17beta-dihydroxyestr-4-en-3-one), 4-Hydroxy-androst-4-ene-3,17-dione 4000 III N Listed as 4-Hydroxy-androst-4-ene-3,17-dione[3,2-c]pyrazole-5α-androstan-17β-ol, 4-Hydroxytestosterone (4,17beta-dihydroxyandrost-4-en-3-one), 5 -Androstenediol (3beta,17beta-dihydroxy-androst-5-ene), 5-Androstenedione (androst-5-en-3,17-dione), 5α-Androstan-3,6,17-trione, 6-bromo-androsta-1,4-diene-3,17-dione, 6-bromo-androstan-3,17-dione, 6α-Methyl-androst-4-ene-3,17-dione, Amobarbital, Amobarbital, Anabolic steroids, Androstanedione (5alpha-androstan-3,17-dione), Aprobarbital, Barbituric acid derivative, Benzphetamine, Bolasterone (7alpha,17alpha-dimethyl-17beta-hydroxyandrost-4-en-3-one), Boldenone (17beta-hydroxyandrost-1,4-diene-3-one), dehydrotestosterone, Boldione, Buprenorphine, Butabarbital (secbutabarbital), Butalbital, Butobarbital (butethal), Calusterone (7beta,17alpha-dimethyl-17beta-hydroxyandrost-4-en-3-one), Chlorhexadol, Chlorphentermine, Clortermine, Clostebol (4-chloro-17beta-hydroxyandrost-4-en-3-one), Codeine & isoquinoline alkaloid, Codeine combination product, Dehydrochloromethyltestosterone (4-chloro-17beta-hydroxy-17alpha-methylandrost-1,4-dien-3-one), Delta1-dihydrotestosterone (17beta-hydroxy-5alpha-4000 CIII N 1-Testosterone, androst-1-en-3-one), Desoxymethyltestosterone, Dihydrocodeine, Dronabinol CIII, Drostanolone (17beta-hydroxy-2alpha-methyl-5alpha-androstan-3-one), Embutramide, Estra-4,9,11-triene-3,17-dione, Ethylestrenol (17alpha-ethyl-17beta-hydroxyestr-4-ene), Ethylmorphine combination product, Fluoxymesterone (9-fluoro-17alpha-methyl-, Anadroid-F, Halotestin, Ora-Testryl 11beta,17beta-dihydroxyandrost-4-en-3-one), Formebolone (2-formyl-17alpha-methyl-, Esiclene, Hubernol 11alpha, 17beta-dihydroxyandrost-1,4-dien-3-one), Furazabol (17alpha-methyl-17beta-hydroxyandrostano[2,3-c]-furazan), Gamma Hydroxybutyric Acid preparations, Ketamine, Lysergic acid, LSD precursor, Lysergic acid amide, LSD precursor, Mestanolone (17alpha-methyl-17beta-hydroxy-5alpha), Mesterolone, Methandienone (17alpha-methyl-17betahydroxyandrost-1,4-dien-3-one), Methandriol (17alpha-methyl-3beta, 17betadihydroxyandrost-5-ene), Methasterone (2alpha,17alpha-dimethyl-5alphaandrostan-17beta-ol-3-one), 2α,17α-dimethyl-17β-hydroxy-5αandrostan-3-one, Methenolone (1-methyl-17beta-hydroxy-5alpha-androst1-en-3-one), Methyldienolone (17alpha-methyl-17beta-hydroxyestra4, 9(10)-dien-3-one), Methyltestosterone (17alpha-methyl-17betahydroxyandrost-4-en-3 -one), Methyltrienolone (17alpha-methyl-17beta-hydroxyestra4, 9,11-trien-3-one), Methyprylon, Mibolerone (7alpha,17alpha-dimethyl-17betahydroxyestr-4-en-3-one), Morphine, Nalorphine, Nalline, Nandrolone (17beta-hydroxyestr-4-en-3-one) CIII Deca-Durabolin, Durabolin, Durabolin-50, Norbolethone (13beta,17alpha-diethyl-17betahydroxygon-4-en-3-one), Norclostebol (4-chloro-17beta-hydroxyestr-4-en-3-one CIII, Anabol-4-19, Lentabol Norethandrolone (17alpha-ethyl-17beta-hydroxyestr-4-, en-3-one), Normethandrolone (17alpha-methyl-17betahydroxyestr-4-en-3-one), Opium combination product 25 mg/du Paregoric, other combination products, Oxandrolone (17alpha-methyl-17beta-hydroxy-2-oxa5alpha-androstan-3-one), Oxymesterone (17alpha-methyl-4,17betadihydroxyandrost-4-en-3-one), Oxymetholone (17alpha-methyl-2-hydroxymethylene17beta-hydroxy-5alpha-androstan-3 -one), Pentobarbital & noncontrolled active ingred., Perampanel Fycompa, [2-(2-oxo-1-phenyl5-pyridin-2-yl-1,2-dihydropyridin-3-yl) benzonitrile], Phendimetrazine, Prostanozol (17beta-hydroxy-5alpha-androstano[3,2-c]pyrazole), Prostanozol; [3,2-c]pyrazole-5α-androstan-17β-ol, Secobarbital & noncontrolled active ingred, Stanozolol (17alpha-methyl-17beta-hydroxy-5alphaandrost-2-eno[3,2-c]-pyrazole), Stenbolone (17beta-hydroxy-2-methyl--5alpha-androst-1-en-3-one), Sulfondiethylmethane, Sulfonethylmethane, Sulfonmethane, Talbutal, Testolactone (13-hydroxy-3-oxo-13,17-secoandrosta-1,4-dien-17-oic acid lactone), Testosterone (17beta-hydroxyandrost-4-en-3-one), Tetrahydrogestrinone (13beta,17alpha-diethyl-17beta-THGhydroxygon-4,9,11-trien-3-one), Thiamylal, Thiopental, Tiletamine & Zolazepam Combination Product, Trenbolone (17beta-hydroxyestr-4,9,11-trien-3-one), Vinbarbital, Alfaxalone, 5α-pregnan-3α-ol-11,20-dione, Alprazolam, Barbital, Bromazepam, Butorphanol, Camazepam, Carisoprodol, Cathine, (+)-norpseudoephedrine, Chloral betaine, Chloral hydrate, Chlordiazepoxide, Clobazam, Clonazepam, Clorazepate, Clotiazepam, Cloxazolam, Delorazepam, Dexfenfluramine, Dextropropoxyphene all dosage forms, Propoxyphene, Diazepam, Dichloralphenazone, Dichloralantipyrine, Diethylpropion, Difenoxin, Eluxadoline, Estazolam, Ethchlorvynol, Ethinamate, Ethyl loflazepate, Fencamfamin, Fenfluramine, Fenproporex, Fludiazepam, Flunitrazepam, Flurazepam, Fospropofol, Halazepam, Haloxazolam, Ketazolam, Loprazolam, Lorazepam, Lorcaserin, Lormetazepam, Mazindol, Mebutamate, Medazepam, Mefenorex, Meprobamate, Methohexital, Methylphenobarbital (mephobarbital), Midazolam, Modafinil, Nimetazepam, Nitrazepam, Nordiazepam, Oxazepam, Oxazolam, Paraldehyde, Pemoline, Pentazocine, Petrichloral, Pentaerythritol chloral, Phenobarbital, Phentermine, Pinazepam, Pipradrol, Prazepam, Quazepam, Sibutramine, SPA, 1-dimethylamino-1,2-diphenylethane, Suvorexant, MK-4305, Temazepam, Tetrazepam, Tramadol, Triazolam, Zaleplon, Zolpidem, Zopiclone, Approved Cannabidiol Drugs (as defined in 21 CFR 7367 V N A drug product in finished dosage formulation that has, 1308.15(f)) been approved by the U.S. Food and Drug Administration, that contains Cannabidiol derived from cannabis and no, more than 0.1 percent (w/w) residual, tetrahydrocannabinols., Brivaracetam, Codeine preparations, Difenoxin, Dihydrocodeine preparations, Diphenoxylate preparations 2.5 mg/25 ug AtSO4, Ethylmorphine preparations, Ezogabine, Lacosamide, Opium preparations, Pregabalin, and Pyrovalerone.

In some embodiments, the bitter active ingredient comprises Anastrozole. In some embodiments, the compositions comprise about 0.1 mg to about 2 mg of Anastrozole.

In some embodiments, the bitter active ingredient comprises Clomiphene Citrate. In some embodiments, the compositions comprise about 0.25 mg to about 15 mg of Clomiphene Citrate.

In some embodiments, the bitter active ingredient comprises Acetaminophen. In some embodiments, the compositions comprise about 25 mg to about 250 mg of Acetaminophen.

In some embodiments, the bitter active ingredient comprises Acyclovir. In some embodiments, the compositions comprise about 25 mg to about 165 mg of Acyclovir.

In some embodiments, the bitter active ingredient comprises Amphotericin B. In some embodiments, the compositions comprise about 5 mg to about 125 mg of Amphotericin B.

In some embodiments, the bitter active ingredient comprises Apomorphine. In some embodiments, the compositions comprise about 0.5 mg to about 11 mg of Apomorphine.

In some embodiments, the bitter active ingredient comprises Benzocaine. In some embodiments, the compositions comprise about 0.5% to about 6% (by weight) of Benzocaine.

In some embodiments, the bitter active ingredient comprises Amlodipine. In some embodiments, the compositions comprise about 1 mg to about 22 mg of Amlodipine.

In some embodiments, the bitter active ingredient comprises Buprenorphine. In some embodiments, the compositions comprise about 0.1 mg to about 13 mg of Buprenorphine.

In some embodiments, the bitter active ingredient comprises Carbamazepine. In some embodiments, the compositions comprise about 25 mg to about 225 mg of Carbamazepine.

In some embodiments, the bitter active ingredient comprises Clonazepam. In some embodiments, the compositions comprise about 0.1 mg to about 3 mg of Clonazepam.

In some embodiments, the bitter active ingredient comprises Chorionic Gonadotropin. In some embodiments, the compositions comprise about 25 U to about 500 U of Chorionic Gonadotropin.

In some embodiments, the bitter active ingredient comprises Clotrimazole. In some embodiments, the compositions comprise about 2 mg to about 21 mg of Clotrimazole.

In some embodiments, the bitter active ingredient comprises Codeine Phosphate. In some embodiments, the compositions comprise about 1 mg to about 35 mg of Cyclobenazaprine.

In some embodiments, the bitter active ingredient comprises Cyclobenazaprine. In some embodiments, the compositions comprise about 1 mg to about 18 mg of Cyclobenazaprine.

In some embodiments, the bitter active ingredient comprises Dextromethorphan. In some embodiments, the compositions comprise about 5 mg to about 35 mg of Dextromethorphan.

In some embodiments, the bitter active ingredient comprises Diazepam. In some embodiments, the compositions comprise about 0.5 mg to about 16 mg of Diazepam.

In some embodiments, the bitter active ingredient comprises Dehydroepiandrosterone (DHEA). In some embodiments, the compositions comprise about 2 mg to about 35 mg of DHEA.

In some embodiments, the bitter active ingredient comprises Diethylstilbestrol. In some embodiments, the compositions comprise about 0.5 mg to about 5 mg of Diethylstilbestrol.

In some embodiments, the bitter active ingredient comprises Diphenhydramine. In some embodiments, the compositions comprise about 1 mg to about 110 mg of Diphenhydramine.

In some embodiments, the bitter active ingredient comprises Metoclopramide. In some embodiments, the compositions comprise about 1 mg to about 22 mg of Metoclopramide.

In some embodiments, the bitter active ingredient comprises Dihydroergotamine. In some embodiments, the compositions comprise about 1 mg to about 15 mg of Dihydroergotamine.

In some embodiments, the bitter active ingredient comprises Doxylamine succinate. In some embodiments, the compositions comprise about 1 mg to about 22 mg of Doxylamine succinate.

In some embodiments, the bitter active ingredient comprises Epigallocatechin gallate (EGCg). In some embodiments, the compositions comprise about 25 mg to about 75 mg of EGCg.

In some embodiments, the bitter active ingredient comprises Estradiol. In some embodiments, the compositions comprise about 0.1 mg to about 2 mg of Estradiol

In some embodiments, the bitter active ingredient comprises Erythromycin. In some embodiments, the compositions comprise about 50 mg to about 250 mg of Erythromycin.

In some embodiments, the bitter active ingredient comprises Ephedrine Sulfate. In some embodiments, the compositions comprise about 1 mg to about 10 mg of Ephedrine Sulfate.

In some embodiments, the bitter active ingredient comprises Atropine Sulfate. In some embodiments, the compositions comprise about 0.1 mg to about 2 mg of Atropine Sulfate.

In some embodiments, the bitter active ingredient comprises Ergotamine Tartrate. In some embodiments, the compositions comprise about 0.5 mg to about 6 mg of Ergotamine Tartrate.

In some embodiments, the bitter active ingredient comprises Estriol. In some embodiments, the compositions comprise about 0.1 mg to about 2 mg of Estriol.

In some embodiments, the bitter active ingredient comprises Progesterone. In some embodiments, the compositions comprise about 25 mg to about 250 mg of Progesterone.

In some embodiments, the bitter active ingredient comprises Estrone. 0 In some embodiments, the compositions comprise about 0.1 mg to about 2.5 mg of Estrone.

In some embodiments, the bitter active ingredient comprises Fentanyl. In some embodiments, the compositions comprise about 1 mcg to about 325 mcg of Fentanyl.

In some embodiments, the bitter active ingredient comprises Guaifenesin. In some embodiments, the compositions comprise about 25 mg to about 200 mg of Guaifenesin.

In some embodiments, the bitter active ingredient comprises Haloperidol. In some embodiments, the compositions comprise about 0.25 mg to about 3 mg of Haloperidol.

In some embodiments, the bitter active ingredient comprises Dexamethasone. In some embodiments, the compositions comprise about 0.25 mg to about 3 mg of Dexamethasone.

In some embodiments, the bitter active ingredient comprises Hydrocodone Bitartrate. In some embodiments, the compositions comprise about 1 mg to about 25 mg of Hydrocodone Bitartrate.

In some embodiments, the bitter active ingredient comprises Hydromorphone hydrochloride. In some embodiments, the compositions comprise about 0.5 mg to about 12 mg of Hydromorphone hydrochloride.

In some embodiments, the bitter active ingredient comprises Hydroxyzine Pamoate. In some embodiments, the compositions comprise about 10 mg to about 30 mg of Hydroxyzine Pamoate.

In some embodiments, the bitter active ingredient comprises Hydroxyzine hydrochloride. In some embodiments, the compositions comprise about 5 mg to about 25 mg of Hydroxyzine hydrochloride.

In some embodiments, the bitter active ingredient comprises Hyoscyamine Sulfate. In some embodiments, the compositions comprise about 0.125 mg to about 1 mg of Hyoscyamine Sulfate.

In some embodiments, the bitter active ingredient comprises Ibuprofen. In some embodiments, the compositions comprise about 50 mg to about 300 mg of Ibuprofen.

In some embodiments, the bitter active ingredient comprises Ketamine. In some embodiments, the compositions comprise about 10 mg to about 250 mg of Ketamine.

In some embodiments, the bitter active ingredient comprises Lidocaine. In some embodiments, the compositions comprise about 1% to about 5% (by weight) of Lidocaine.

In some embodiments, the bitter active ingredient comprises Lorazepam. In some embodiments, the compositions comprise about 0.5 mg to about 4 mg of Lorazepam.

In some embodiments, the bitter active ingredient comprises Methadone. In some embodiments, the compositions comprise about 1 mg to about 30 mg of Methadone.

In some embodiments, the bitter active ingredient comprises Methocarbamol. In some embodiments, the compositions comprise about 100 mg to 300 mg of Methocarbamol.

In some embodiments, the bitter active ingredient comprises Methscopolamine Bromide. In some embodiments, the compositions comprise about 0.125 mg to about 2 mg of Methscopolamine Bromide.

In some embodiments, the bitter active ingredient comprises Methylprednisolone. In some embodiments, the compositions comprise about 1 mg to about 5 mg of Methylprednisolone.

In some embodiments, the bitter active ingredient comprises Methyltestosterone. In some embodiments, the compositions comprise about 2 mg to about 20 mg of Methyltestosterone.

In some embodiments, the bitter active ingredient comprises Miconazole Nitrate. In some embodiments, the compositions comprise about 5 mg to about 250 mg of Miconazole Nitrate.

In some embodiments, the bitter active ingredient comprises Morphine Sulfate. In some embodiments, the compositions comprise about 1 mg to about 35 mg of Morphine Sulfate.

In some embodiments, the bitter active ingredient comprises Prochlorperazine. In some embodiments, the compositions comprise about 2 mg to about 22 mg of Prochlorperazine.

In some embodiments, the bitter active ingredient comprises Nicotine. In some embodiments, the compositions comprise about 0.5 mg to about 6 mg of Nicotine.

In some embodiments, the bitter active ingredient comprises Nifedipine. In some embodiments, the compositions comprise about 5 mg to about 15 mg of Nifedipine.

In some embodiments, the bitter active ingredient comprises Oxycodone. In some embodiments, the compositions comprise about 1 mg to about 38 mg of Oxycodone.

In some embodiments, the bitter active ingredient comprises Piroxicam. In some embodiments, the compositions comprise about 5 mg to about 28 mg of Piroxicam.

### Methods of Use:

Sexual dysfunction is experienced by both men and women. The sexual response cycle has four phases: excitement, plateau, orgasm, and resolution. Sexual dysfunction can be caused by physical and emotional factors, or a combination of both. The side effects of some medications also can lead to sexual dysfunction.

Sildenafil and sildenafil citrate, sold as the brand name Viagra®, among others, is a medication used to treat erectile dysfunction and pulmonary arterial hypertension. Other pharmaceuticals that operate by the same mechanism include tadalafil (Cialis®) and vardenafil (Levitra®). Sildenafil, tadalfil, and vardenafil act by inhibiting cGMP-specific phosphodiesterase type 5 (phosphodiesterase 5, PDE5), an enzyme that promotes degradation of cGMP, which regulates blood flow.

Approximately 52% of men have experienced some form of sexual dysfunction. The prominence of female sexual dysfunction has been documented, as well. Estimates have shown that over 100 million women have never reached a successful orgasm, or have lack of interest in and motivation for sexual desire. When taken orally by mouth (e.g. tablets), sildenafil for erectile dysfunction results in an average time to onset of erections of 27 minutes (ranging from 20 to 70 minutes).

Current sexual dysfunction pharmaceutical manufacturers have been unable to successfully develop sub-lingual, or under the tongue, dosage forms due to a severe bitter taste of the compound. Additionally, many patients have difficulty swallowing—some studies show that only 40% of patients can swallow pills. Additionally, oral dosage forms are affected by gastric emptying time and the type of meal consumed. This, in turn, affects the onset of the effects of the pharmaceuticals. Because sex is a spontaneous activity, or an activity where performance is often required quickly, the delay in onset of pharmaceutical effects can have negative consequences on the sexual activity. Accordingly, there is a need for dosage forms of sildenafil and tadalafil, which do not have a bitter taste and which have a faster time of onset to desired sexual effects.

There are generally four categories of sexual dysfunction: (1) Desire disorders: The lack of sexual desire or interest in sex; (2) Arousal disorders: Unable to become physically aroused during sexual activity, including problems achieving and maintaining an erection (erectile dysfunction); (3) Orgasm disorders: The delay or absence of orgasm (climax); and (4) Pain disorders: Pain during intercourse (this mainly affects women).

Often, the main culprits contributing to sexual dysfunction are: smoking, obesity, alcohol, heart disease, diabetes, high blood pressure, and high cholesterol. An imbalance of hormones such as low testosterone and oxytocin may also play role in lack of interest and inability to maintain an erection.

Medications and over-the-counter pharmaceuticals can affect libido (desire) and others can affect the ability to become aroused or achieve orgasm. Such medications include, but are not limited to, tricyclic antidepressants, monoamine oxidase inhibitors (MAOIs), antipsychotic medications, anti-mania medications, and selective serotonin reuptake inhibitors (SSRIs).

Some medications make cause erectile dysfunction, including, but not limited to, anti-hypertensive medications (used to treat high blood pressure), diuretics, centrally acting agents, a-Adrenergic blockers, and b-adrenergic (beta) blockers.

The risk of sexual side effects is increased when an individual is taking multiple medications.

It is estimated that about 52% of men suffer from a degree of erectile dysfunction at some point in their life. The female population has been documented in a variety of articles. Estimates have shown over 100 million who never reach a successful orgasm, lack interest and motivation for sexual desire.

Viagra® (sildenafil) and Cialis® (tadalafil) tablets are the most commonly prescribed medications to reduce sexual dysfunction and enhance sexual performance. Both products are licensed for erectile dysfunction in men. There is no licensed medication to treat sexual dysfunction in women, although there have been some small trials with sildenafil.

Sildenafil and tadalafil work by causing vasodilation, which increases the blood flow to the penis to help a man get and sustain an erection. They work only when the man is sexually aroused or stimulated. Sildenafil should be taken about 60 minutes before sexual activity and tadalafil about 30 minutes before. They should not be taken more than once a day. Sildenafil and tadalafil usually start to work within about 30-60 minutes, depending on meal and gastric emptying time.

The length of time sildenafil and tadalfil will last may vary from person to person. However, most men find that the effects of sildenafil will last for 2-3 hours after taking the tablet, and tadalafil will last for 24-36 hours for multiple encounters.

Whether sildenafil/tadalafil are suitable for an individual and the respective dose will depend upon assessment by a doctor or pharmacist of the health and other medication(s) that the individual may be taking.

When a chemical comes in contact with the mucous membrane beneath the tongue, it is absorbed. Because the connective tissue beneath the epithelium contains a profusion of capillaries, the substance then diffuses into them and enters the venous circulation. In contrast, substances absorbed in the intestines are subject to first-pass metabolism in the liver before entering the general circulation.

In some embodiments, sublingual administration has certain advantages over oral administration. For example, sublingual administration is more direct, so it is often faster and it ensures that the substance will risk degradation only by salivary enzymes before entering the bloodstream, whereas orally administered pharmaceuticals must survive passage through the hostile environment of the gastrointestinal tract, which risks degrading them, by either stomach acid or bile, or by enzymes such as monoamine oxidase (MAO). Furthermore, after absorption from the gastrointestinal tract, such pharmaceuticals must pass to the liver, where they may be extensively altered; this is known as the first pass effect of pharmaceutical metabolism. Due to the digestive activity of the stomach and intestines, the oral route is unsuitable for certain substances.

In some embodiments, the present disclosure provides methods of treating erectile dysfunction comprising administering a therapeutically effective amount of a composition of the present disclosure comprising sildenafil to a patient in need thereof.

In some embodiments, the administered composition comprises about 20 mg of sildenafil or a pharmaceutically acceptable salt thereof. In some embodiments, the administered composition comprises about 40 mg of sildenafil or a pharmaceutically acceptable salt thereof.

In some embodiments, the administered composition comprises:
about 2 wt. % to about 6 wt. % of sildenafil or a pharmaceutically acceptable salt thereof,
about 1 wt. % to about 5 wt. % of a bitterness reducing agent; and
about 6 wt. % to about 10 wt. % of a sweetener or flavoring agent.

In some embodiments, the administered composition comprises:
about 3 wt. % to about 5 wt. % of sildenafil or a pharmaceutically acceptable salt thereof,
about 2 wt. % to about 4 wt. % of a bitterness reducing agent; and
about 7 wt. % to about 9 wt. % of a sweetener or flavoring agent.

In some embodiments, the administered composition comprises:
about 4 wt. % of sildenafil or a pharmaceutically acceptable salt thereof,
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, silica, acacia, chocolate, marshmallow, vanilla and spearmint.

In some embodiments, the administered composition comprises:
about 3 wt. % to about 5 wt. % of sildenafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of silica,
about 0.25 wt % to about 0.75 wt. % of acacia,
about 1 wt % to about 4 wt. % of chocolate,
about 1 wt % to about 4 wt. % of marshmallow,
about 0.4 wt % to about 0.8 wt. % of vanilla and
about 0.5 wt % to about 1.5 wt. % of spearmint.

In some embodiments, the administered composition comprises:
about 4 wt. % of sildenafil or a pharmaceutically acceptable salt thereof,
about 3 wt. % NF01,
about 0.5 wt. % acesulfame,
about 0.5 wt. % steviol,
about 0.5 wt. % silica,
about 0.5 wt. % acacia,
about 3 wt. % chocolate,
about 3 wt. % marshmallow,
about 0.6 wt. % vanilla and
about 1 wt. % spearmint.

In some embodiments, the present disclosure provides methods of treating erectile dysfunction comprising administering a therapeutically effective amount of a composition of the present disclosure comprising tadalafil to a patient in need thereof.

In some embodiments, the present disclosure provides methods of treating erectile dysfunction comprising administering a therapeutically effective amount of a composition of the present disclosure comprising tadalafil and sildenafil to a patient in need thereof.

In some embodiments, the administered composition comprises 100 mg of sildenafil or a pharmaceutically acceptable salt thereof and 20 mg of tadalafil or a pharmaceutically acceptable salt thereof. In some embodiments, the administered composition comprises 50 mg of sildenafil or a pharmaceutically acceptable salt thereof and 5 mg of tadalafil or a pharmaceutically acceptable salt thereof.

In some embodiments, the administered composition comprises:
about 0.2 wt. % to about 1.8 wt. % of sildenafil or a pharmaceutically acceptable salt thereof;
about 0.05 wt. % to about 0.35 wt. % of tadalafil or a pharmaceutically acceptable salt thereof;
about 1 wt. % to about 5 wt. % of a bitterness reducing agent; and
about 4 wt. % to about 8 wt. % of a sweetener or flavoring agent.

In some embodiments, the administered composition comprises:
about 0.5 wt. % to about 1.5 wt. % of sildenafil or a pharmaceutically acceptable salt thereof;
about 0.1 wt. % to about 0.3 wt. % of tadalafil or a pharmaceutically acceptable salt thereof;
about 2 wt. % to about 4 wt. % of a bitterness reducing agent; and
about 5 wt. % to about 7 wt. % of a sweetener or flavoring agent.

In some embodiments, the administered composition comprises:
about 1 wt. % of sildenafil or a pharmaceutically acceptable salt thereof,
about 0.2 wt. % of tadalafil or a pharmaceutically acceptable salt thereof
about 3 wt. % of a bitterness reducing agent; and
about 6 wt. % of a sweetener or flavoring agent.

In some embodiments, the bitterness reducing agent comprises NF01 and the sweetener or flavoring agent comprises acesulfame, steviol, spearmint, marshmallow, vanilla, chocolate, and acacia.

In some embodiments, the administered composition comprises:
about 0.5 wt. % to about 1.5 wt. % of sildenafil or a pharmaceutically acceptable salt thereof,
about 0.1 wt. % to about 0.3 wt. % of tadalafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.5 wt % to about 1.5 wt. % of spearmint,
about 1 wt % to about 3 wt. % of marshmallow,
about 0.1 wt % to about 0.5 wt. % of vanilla,
about 0.1 wt % to about 0.5 wt. % of chocolate, and
about 0.25 wt % to about 0.75 wt. % of acacia.

In some embodiments, the administered composition comprises:
about 1 wt. % of sildenafil or a pharmaceutically acceptable salt thereof,
about 0.2 wt. % of tadalafil or a pharmaceutically acceptable salt thereof,
about 3 wt. % of NF01,
about 0.5% of acesulfame,
about 0.5% of steviol,
about 1 wt. % of spearmint,
about 2 wt. % of marshmallow,
about 0.3 wt % of vanilla,
about 0.3 wt % of chocolate, and
about 0.5% of acacia.

In some embodiments, the present disclosure provides methods of treating erectile dysfunction comprising administering a therapeutically effective amount of a composition of the present disclosure comprising tadalafil to a patient in need thereof.

The disclosure is now described with reference to the following examples. Before describing several exemplary embodiments of the disclosure, it is to be understood that the disclosure is not limited to the details of construction or process steps set forth in the following description. The disclosure is capable of other embodiments and of being practiced or being carried out in various ways.

Sublingual Formulations of Cannabis extracts, CBD and THC and their Use:

Cannabis extracts (as well as purified constituents of these extracts) have widely recognized recreational and medicinal benefits; however, despite this potential, the illegality of cannabis has prevented its widespread use for the treatment of medical conditions. However, as more states and countries legalize cannabis for medical and recreational use, there is a need to develop cannabis extract formulations that address the shortcoming of existing technologies and allow for a fuller exploitation of the medical and recreational potential of these products.

The principal psychoactive constituent of cannabis extract is delta-9 tetrahydrocannabinol (THC). Other constituents of cannabis extract include cannabinoids, such as, for example, cannabidiol (CBD), cannabinol (CBN), tetrahydrocannabivarin (THCV) and cannabigerol (CBG). Many medically-beneficial constituents of cannabis extract (notably CBD and THC) undergo extension first-pass metabolism when administered orally, which limits their oral bioavailability. Thus, formulations that avoid first-pass metabolism (such as sublingual or buccal formulations) would be beneficial. Unfortunately, cannabis extracts (as well as purified constituents of the extracts) are frequently described as bitter tasting and, as such, it is difficult to provide palatable sublingual or buccal dosage forms of these ingredients.

The present disclosure provides palatable sublingual or buccal dosage forms of cannabis extracts as well the constituents of cannabis extracts (such as CBD and THC) that provide rapid onset of these beneficial active ingredients and avoid first-pass metabolism, which limits the bioavailability of the existing orally-administered formulations.

In one aspect, the present disclosure provides sublingual formulations comprising a cannabis extract. In some embodiments, the compositions comprise from about 1 mg to about 50 mg of a cannabis extract, including about 1 mg, about 5 mg, about 10 mg, about 15 mg, about 20 mg, about 25 mg, about 30 mg, about 35 mg, about 40 mg, about 45 mg, and about 50 mg including all ranges there between.

In some embodiments, the compositions comprise about 1 mg, about 5 mg, about 10 mg, about 15 mg, about 20 mg, about 25 mg, about 30 mg, about 35 mg, about 40 mg, about 45 mg, or about 50 mg of cannabis extract.

In some embodiments, the composition comprises:
about 0.2 wt. % to about 1.8 wt. % of a cannabis extract;
about 1 wt. % to about 5 wt. % of a bitterness reducing agent; and
about 6 wt. % to about 10 wt. % of a sweetener or flavoring agent.

In some embodiments, the composition comprises:
about 0.5 wt. % to about 1.5 wt. % of a cannabis extract;
about 2 wt. % to about 4 wt. % of a bitterness reducing agent; and
about 7 wt. % to about 9 wt. % of a sweetener or flavoring agent.

In some embodiments, the composition comprises:
about 1 wt. % of a cannabis extract
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, acacia, silica, menthol, chocolate, marshmallow, spearmint and vanilla.

In some embodiments, the composition comprises:
about 0.5 wt. % to about 1.5 wt. % of a cannabis extract,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of acacia,
about 0.25 wt % to about 0.75 wt. % of silica,
about 0.1 wt % to about 0.4 wt. % of menthol,
about 2 wt % to about 4 wt. % of chocolate,
about 2 wt % to about 4 wt. % of marshmallow,
about 0.3 wt % to about 0.5 wt. % of spearmint, and
about 0.5 wt % to about 0.7 wt. % of vanilla.

In some embodiments, the composition comprises:
about 1 wt. % of a cannabis extract,
about 3 wt. % of NF01,
about 0.5 wt. % of acesulfame,
about 0.5 wt. % of steviol,
about 0.5 wt. % of acacia,
about 0.5 wt. % of silica,
about 0.3 wt. % of menthol,
about 3 wt. % of chocolate,
about 3 wt. % of marshmallow,
about 0.4 wt % of spearmint, and
about 0.6 wt % of vanilla.

In some embodiments, the cannabis extract comprises of one or more of the following:
(i) THC;
(ii) Tetrahydrocannabinolic acid (THC-A);
(iii) CBD with a THC content less than or equal to 0.3 mg/g;
(iv) CBD in combination with THC in a 2% to 6% ratio;
(v) CBD in combination with THC-A in a 2% to 6% ratio; and
(iv) Delta-8 Tetrahydrocannabinol.

In some embodiments, the cannabis extract comprises of one or more of the following:
(i) THC in doses ranging from 0.1 mg to 240 mg (0.01% to 25.26% by weight);
(ii) THC-A in doses of 0.1 mg to 240 mg (0.01% to 25.26% by weight);
(iii) CBD in doses of 0.1 mg to 240 mg with a THC content less than or equal to 0.3 mg (making this dosage form legal in all states of the United States); and
(iv) CBD in doses of 0.1 mg to 240 mg (0.01% to 21.26% by weight) in combination with THC in a 53:1 ratio (CBD:THC), or down to a ratio of 0.001:1 (CBD:THC), of Delta-9 THC in the decarboxylated and non-decarboxylated forms at specific temperatures.

In some embodiments, the cannabis extract is obtained from one or more of C. sativa, C. indica, C. ruderalis and hybrids thereof.

In some embodiments, the cannabis extract comprises from about 24,000:1 CBD:THC (i.e., 240 mg CBD to 0.01 mg THC) to about 1:24,000 CBD:THC (i.e., 0.01 mg CBD to 240 mg THC). In some embodiments, the cannabis extract comprises from about 200,000:1 CBD:THC to about 1:200,000 CBD:THC. In some embodiments, the cannabis extract comprises at least 99 wt. % of CBD. In some embodiments, the cannabis extract comprises from about 99 wt. % to about 99.9 wt. % of CBD. In some embodiments, the cannabis extract consists essentially of one or more of the following: cannabinoids, terpenes and flavonoids. In some embodiments, the cannabis extract consists essentially of one or more of the following: cannabinoids, terpenes and flavonoids.

In some embodiments, the cannabis extract comprises a cannabinoid selected from the group consisting of delta-9-tetrahydrocannabinol, tetrahydrocannabivarin, cannabidiol, Cannabidivarin, cannabigerol, Cannabichromene, Cannabinol, tetrahydrocannabinolic acid, cannabidiolic acid, cannabigerolic acid and mixtures thereof. In some embodiments, the cannabis extract comprises a cannabinoid selected from the group consisting of cannabidiol, Cannabidivarin, and Cannabigerol and mixtures thereof.

In some embodiments, the cannabis extract comprises a terpene selected from the group consisting of myrcene, isopulegol, menthol, Nerolidol-trans, A-bisabolol, linalool, β-caryophyllene, caryophyllene oxide, guaiol, Humulene, and eucalyptol and mixtures thereof.

In one aspect, the present disclosure provides sublingual formulations comprising cannabidiol or a pharmaceutically acceptable salt, ester, prodrug, or solvate thereof (“CBD”). In some embodiments, the compositions from about 0.1 mg to about 240 mg of CBD or a pharmaceutically acceptable salt thereof, including about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, and about 240 mg including all ranges there between.

In some embodiments, the compositions comprise about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, or about 240 mg of CBD or a pharmaceutically acceptable salt thereof.

In some embodiments, the composition comprises:
about 0.2 wt. % to about 1.8 wt. % of cannabidiol or a pharmaceutically acceptable salt, ester, prodrug, or solvate thereof;
about 1 wt. % to about 5 wt. % of a bitterness reducing agent; and
about 6 wt. % to about 10 wt. % of a sweetener or flavoring agent.

In some embodiments, the composition comprises:
about 0.5 wt. % to about 1.5 wt. % of cannabidiol or a pharmaceutically acceptable salt, ester, prodrug, or solvate thereof, ester, prodrug, or solvate thereof;
about 2 wt. % to about 4 wt. % of a bitterness reducing agent; and
about 7 wt. % to about 9 wt. % of a sweetener or flavoring agent.

In some embodiments, the composition comprises:
about 1 wt. % of cannabidiol or a pharmaceutically acceptable salt, ester, prodrug, or solvate thereof,
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, acacia, silica, menthol, chocolate, marshmallow, spearmint and vanilla.

In some embodiments, the composition comprises:
about 0.5 wt. % to about 1.5 wt. % of cannabidiol or a pharmaceutically acceptable salt, ester, prodrug, or solvate thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of acacia,
about 0.25 wt % to about 0.75 wt. % of silica,
about 0.1 wt % to about 0.4 wt. % of menthol,
about 2 wt % to about 4 wt. % of chocolate,
about 2 wt % to about 4 wt. % of marshmallow,
about 0.3 wt % to about 0.5 wt. % of spearmint, and
about 0.5 wt % to about 0.7 wt. % of vanilla.

In some embodiments, the composition comprises:
about 1 wt. % of cannabidiol or a pharmaceutically acceptable salt, ester, prodrug, or solvate thereof,
about 3 wt. % of NF01,
about 0.5 wt. % of acesulfame,
about 0.5 wt. % of steviol,
about 0.5 wt. % of acacia,
about 0.5 wt. % of silica,
about 0.3 wt. % of menthol,
about 3 wt. % of chocolate,
about 3 wt. % of marshmallow,
about 0.4 wt % of spearmint, and
about 0.6 wt % of vanilla.

In one aspect, the present disclosure provides sublingual formulations comprising THC. In some embodiments, the bitter active ingredient is tetrahydrocannabinol or a pharmaceutically acceptable salt, ester, prodrug, or solvate thereof (THC).

In some embodiments, the compositions from about 0.1 mg to about 240 mg of THC or a pharmaceutically acceptable salt thereof, including about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, and about 240 mg including all ranges there between.

In some embodiments, the compositions comprise about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, or about 240 mg of THC or a pharmaceutically acceptable salt thereof.

In some embodiments, the composition comprises:
about 0.2 wt. % to about 1.8 wt. % tetrahydrocannabinol or a pharmaceutically acceptable salt, ester, prodrug, or solvate thereof;
about 1 wt. % to about 5 wt. % of a bitterness reducing agent; and
about 6 wt. % to about 10 wt. % of a sweetener or flavoring agent.

In some embodiments, the composition comprises:
about 0.5 wt. % to about 1.5 wt. % of tetrahydrocannabinol or a pharmaceutically acceptable salt, ester, prodrug, or solvate thereof;
about 2 wt. % to about 4 wt. % of a bitterness reducing agent; and
about 7 wt. % to about 9 wt. % of a sweetener or flavoring agent.

In some embodiments, the composition comprises:
about 1 wt. % of tetrahydrocannabinol or a pharmaceutically acceptable salt, ester, prodrug, or solvate thereof,
about 3 wt. % of a bitterness reducing agent; and
about 8 wt. % of a sweetener or flavoring agent.

In some embodiments, the bitterness reducing agent comprises NF01 and the sweetener or flavoring agent comprises acesulfame, steviol, acacia, silica, menthol, chocolate, marshmallow, spearmint and vanilla.

In some embodiments, the composition comprises:
about 0.5 wt. % to about 1.5 wt. % of tetrahydrocannabinol or a pharmaceutically acceptable salt, ester, prodrug, or solvate thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of acacia,
about 0.25 wt % to about 0.75 wt. % of silica,
about 0.1 wt % to about 0.4 wt. % of menthol,
about 2 wt % to about 4 wt. % of chocolate,
about 2 wt % to about 4 wt. % of marshmallow,
about 0.3 wt % to about 0.5 wt. % of spearmint, and
about 0.5 wt % to about 0.7 wt. % of vanilla.

In some embodiments, the composition comprises:
about 1 wt. % of tetrahydrocannabinol or a pharmaceutically acceptable salt, ester, prodrug, or solvate thereof,
about 3 wt. % of NF01,
about 0.5 wt. % of acesulfame,
about 0.5 wt. % of steviol,
about 0.5 wt. % of acacia,
about 0.5 wt. % of silica,
about 0.3 wt. % of menthol,
about 3 wt. % of chocolate,
about 3 wt. % of marshmallow,
about 0.4 wt % of spearmint, and
about 0.6 wt % of vanilla.

In one aspect, the present disclosure provides sublingual formulations comprising a mixture of CBD and THC. In some embodiments, the compositions comprise cannabidiol or a pharmaceutically acceptable salt, ester, prodrug, or solvate thereof and tetrahydrocannabinol or a pharmaceutically acceptable salt, ester, prodrug, or solvate thereof. In some embodiments, the ratio of CBD to THC is about 1:1.

In some embodiments, the compositions comprise from about 0.1 mg to about 240 mg of CBD, including about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, and about 240 mg including all ranges there between and from about 1 mg to about 240 mg of THC, including about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, and about 240 mg including all ranges there between.

In some embodiments, the compositions comprise about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, or about 240 mg of CBD and about 0.1 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, or about 240 mg of THC.

The cannabis extract, CBD and THC-containing compositions described herein are useful for the treatment and prevention of a wide range of disorders, including, for example, inflammatory bowel disease (IBS), Crohn's disease (CD), irritable bowel syndrome (IBS), ulcerative colitis (UC), nausea, vomiting, anorexia, cachexia, all forms of pain (i.e. acute, chronic, neuropathic, etc.), gastrointestinal tract distress (i.e. heartburn, indigestion, stomachache, etc.), migraine headaches, postmenstrual syndrome (PMS), Cancer, neurodegenerative diseases like Lou Gehrig's disease, Huntington's disease, Alzheimer's dementia, Parkinson's disease and Parkinsonian-type symptoms, spinal-cord injuries; HIV/AIDS, agitation, insomnia, depression, muscle spasms, spasticity from multiple sclerosis, glaucoma, Autism Spectrum Disorder (ASD), Attention Deficit Hyperactivity Disorder (ADHD), Post-Traumatic Stress Disorder (PTSD), and anxiety disorders.

In some embodiments, the present disclosure provides methods of treating a disorder selected from inflammation and pain comprising administering a therapeutically effective amount of a cannabis extract, CBD and/or THC containing compositions described herein to a patient in need thereof. In some embodiments, the pain disorder is selected from the group consisting of pain disorder is selected from the group consisting of acute, chronic, neuropathic or migraine headache pain.

In some embodiments, the present disclosure provides methods of treating a disorder selected from insomnia, post-traumatic stress disorder, and anxiety comprising administering a therapeutically effective amount of a cannabis extract, CBD and/or THC containing compositions described herein to a patient in need thereof.

In some embodiments, the present disclosure provides methods of treating a disorder selected from Parkinson's disease, Alzheimer's disease, Autism Spectrum Disorder, and seizures comprising administering a therapeutically effective amount of a cannabis extract, CBD and/or THC containing compositions described herein to a patient in need thereof.

### EXAMPLES

The applicants have discovered that the specific selection of bitterness reducing agents and sweetener or flavoring agents was much more effective than other, similar bitterness reducing agents and sweetener or flavoring agents. For example, it was found that the effectiveness of a particular sweetener or flavoring agent or bitterness reducing agent depended in part on the commercial source of the ingredient. Therefore, without being bound by any theory, it is hypothesized that the effectiveness of a particular sweetener or flavoring agent or bitterness reducing agent depends on the chemical composition of the ingredient, which varies from manufacturer to manufacturer.

The following examples used the bitterness reducing agent and sweetener or flavoring agents having the chemical compositions represented by the UV/Vis spectra shown in the FIGS. 1-10. Surprisingly, these ingredients were found to be particularly effective in the taste-masking dosage forms of the present disclosure.

### Example 1

Melt troche base with gentle heat while stirring using a water bath.

Using a mortar and pestle, triturate the sildenafil citrate, Bitterness reducing agent to a fine powder.

Sift the powder from Step 2 along with Oxytocin into the melted base and stir until evenly dispersed.

Add the Flavors and mix well.

Flip the mold over and spray the mold cavities with PAM, allow excess to drain on a paper towel.

Using an appropriate size syringe, distribute the melted mixture into each cavity of the mold.

Allow to solidify at room temperature

### Example 2. Sildenafil and Tadalafil

Using the process of Example 1, a sublingual formulation was prepared containing the following ingredients

| Ingredient | Amount | |
|---|---|---|
| Sildenafil citrate (Each troche contains | 0.375 | g |
| 0.0125 GM of Sildenafil Citrate) | | |
| Tadalafil (Each troche contains 0.0025 | 0.075 | g |
| GM of Tadalafil) | | |
| Bitterness reducing agent power | 0.98 | g |
| Acesulfame potassium powder | 0.175 | g |
| Steviol glycosides 95% (powder) | 0.175 | g |
| Spearmint oil | 0.35 | mL |
| Marshmallow flavor liquid | 0.7 | mL |
| Vanilla flavor liquid | 0.0875 | mL |
| Chocolate flavor liquid | 0.0875 | mL |
| Acacia NF (spray dried gum Arabic) | 0.175 | g |
| Polyethylene glycol 1450 crystal | 31.612 | g |
| Blue coloring (liquid) | 1 | gtts |

### Example 3. Sildenafil and Oxytocin

Using the process of Example 1, a sublingual formulation was prepared containing the following ingredients:

| Ingredient | Amount | |
|---|---|---|
| Sildenafil citrate (Each troche contains | 0.98 | g |
| 0.028 GM of Sildenafil Citrate) | | |
| Oxytocin (Stock Solution 1 IU/mg) | 0.075 | g |
| Bitterness reducing agent power | 0.98 | g |
| Acesulfame potassium powder | 0.175 | g |
| Steviol glycosides 95% (powder) | 0.175 | g |
| Spearmint oil | 0.35 | mL |
| Marshmallow flavor liquid | 0.7 | mL |
| Vanilla flavor liquid | 0.0875 | mL |
| Chocolate flavor liquid | 0.0875 | mL |
| Acacia NF (spray dried gum Arabic) | 0.175 | g |
| Silica gel micronized powder | 0.175 | g |
| Polyethylene glycol 1450 crystal | 31.612 | g |
| Blue coloring (liquid) | 1 | gtts |

### Example 4. Sildenafil

Troche compositions comprising sildenafil, taste-masking and/or bitterness-masking agents were prepared. The compositions were then sublingually administered to patients and, after administered, the patients responded to a questionnaire that measured how well the composition masked the bitterness of sildenafil.

### Example 4a: Using the process of Example 1, a sublingual formulation was prepared containing the following ingredients:

| | | |
|---|---|---|
| Ingredient | Amount | Dissolving time |

| | | | |
|---|---|---|---|
| PEG 1450 | 33.53 | g | 18 mins |
| Sildenafil | 1.4 | g | |
| Tutti Frutti | 0.35 | ml | |

Patient study results: 4 Subjects were administered the Troche. All Subjects described the taste as “horrible” and “very bitter”. Patients noted that it took too long to dissolve under the tongue. All refused to try it again and said it left a horrible aftertaste in their mouths. Horrible, disgusting, awful were all the main key words to describe the taste. All patients described the composition as extremely bitter.

Example 4b: Using the process of Example 1, a sublingual formulation was prepared containing the following ingredients:

| | | |
|---|---|---|
| Ingredient | Amount | Dissolving time |

| | | |
|---|---|---|
| PEG1450 | 30.38 | 18 mins |

| | | | |
|---|---|---|---|
| Sugar | 1.4 | g | Did Not Help |
| Sildenafil | 1.4 | g | 18 mins |
| PCCA Bitterness Reducing Agent | 1.75 | g | Did not help |
| Spearmint | 0.35 | ml | 18 mins |

Patient study results: 6 Subjects were administered the Troche. All subjects felt described the taste as “horrible” in taste but noted an improved sweetness compared to Example 4a. Composition was described as very bitter and patients said it took too long to dissolve under the tongue. 2 new subjects (in addition to 4 from Example 4a) described as very bitter, tasting horrible and would never want to try it again. All subjects felt bitterness remained for over 35 mins after the troche had dissolved.

Example 4c: Using the process of Example 1, a sublingual formulation was prepared containing the following ingredients:

| Ingredient | Amount | | Dissolving time |
|---|---|---|---|
| PEG1450 | 29.58 | g | 10 mins but jelly, |
| | | | gummy, slimy like |
| Sugar | 1.4 | g | Did Not Help |
| Sildenafil | 1.4 | g | No effect |
| PCCA Bitterness Reducing Agent | 1.75 | g | Did not help |
| Silica | 0.6 | g | Had an effect on |
| | | | dissolve time and |
| | | | helped solubility |
| Acacia | 0.6 | g | Had an effect on |
| | | | dissolve time |
| Winterfresh | 0.35 | ml | No effect |

Patient study results: Troche was administered to ten subjects: two who had been administered the compositions of Examples 4a and 4b and eight subjects with erectile dysfunction who had previously failed treatment with oral Sildenafil. All subjects including the 2 previous subjects felt that the formulations were too gummy/chewy and the semisolid wasn't in good formation to apply under the tongue. The subjects all reported the taste as horrible and bitter. Subjects reported that flavor was worse with Winter Fresh in masking the bitterness.

Example 4d: Using the process of Example 1, a sublingual formulation was prepared containing the following ingredients:

| Ingredient | Amount | | Dissolving time |
|---|---|---|---|
| PEG1450 | 29.84 | | 10 mins but jelly, |
| | | | gummy, slimmy like |
| Sugar | 1.4 | g | No effect |
| Sildenafil | 1.4 | g | No effect |
| Alpha Bitterness Reducing Agent | 1.6 | g | Did not help |
| Silica | 0.4 | g | No effect |
| Acacia | 0.4 | g | No effect |
| Cherry | 0.4 | ml | No effect |

Patient study results: Troche was administered to the eight subjects with erectile dysfunction who had previously failed treatment with oral Sildenafil described in Example 4c. Subjects described the composition as too gummy/chewy and the semisolid not in good formation to apply under the tongue. The subjects described taste as horrible and bitter. Subjects described the flavor as worse with Cherry when compared to spearmint in masking the bitterness.

Example 4e: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 31.44 | g | 10 mins but better |
| Splenda | 1.4 | g | No effect |
| Sildenafil | 1.4 | g | No effect |
| Silica | 0.2 | g | No effect |
| Acacia | 0.2 | g | No effect |
| Watermelon | 0.4 | ml | No effect |

Patient study results: Troche was administered to four subjects with ED. Subjects described that the formulation felt like a semisolid and disintegrated under the tongue in less than 10 minutes. Overall taste was described as very bitter and not less bitter than the formulations from Examples 4a-4d previous trials with the bitterness reducing agents. Subjects described bitter on their palate for over 35 mins after administration. Subjects described the sugar taste was artificial and could easily tell it was like taking Splenda.

Example 4f: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 32.6 | g | 10 mins same |
| Acesulfame | 0.7 | g | No effect |
| Sildenafil | 1.4 | g | No effect |
| Silica | 0.2 | g | No effect |
| Acacia | 0.2 | g | No effect |
| Spearmint | 0.4 | ml | No effect |

Patient study results: Troche was administered to 12 new Subjects. Subjects reported extreme bitterness and described the composition as having a horrible artificial sugar flavor. Subjected positively reported on the scent of spearmint, but the bitterness was too powerful. Subjects stated that they never wanted to try it again.

Example 4₂: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 33.16 | g | 10 mins same |
| Steviol | 0.3 | g | No effect |
| Sildenafil | 1.4 | g | No effect |
| Silica | 0.2 | g | No effect |
| Acacia | 0.2 | g | No effect |
| Spearmint | 0.6 | ml | No effect |

Patient study results: Troche was administered to the 12 Subjects from Example 5f. Subjects reported extreme bitterness and that the spearmint was very strong with some subjects reporting a burning sensation under the tongue. Subjects reported positively on the sugar flavor of sweetness was better compared to Example 5f but the bitterness too high to determine the level of sweetness on the palate.

Example 4h: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 27.04 | g | 10 mins same |
| Steviol | 0.3 | g | No effect |
| Sildenafil | 1.4 | g | No effect |
| KoolAid Mix | 8 | g | No effect |

Patient study results: Troche was administered to the 12 Subjects from Example 5g. Subject described same bitterness as Example 5g, and the Koolaid did not dissolve completely. Conclude that the formulation from a psychical chemistry perspective does not work.

Example 4i: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 32.17 | g | 10 mins same |
| Acesulfame | 0.1 | g | No effect |
| Steviol | 0.1 | g | |
| Regular Sugar | 0.1 | g | |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Spearmint | 0.35 | ml | No effect |

Patient study results: Troche was administered to the 12 Subjects from Example 5h. Subjects described a significant delay in the onset of bitterness. The subjects described the sweetness as artificial and still too sweet.

Example 4j: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 32.25 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Spearmint | 0.35 | ml | No effect |

Patient study results: Troche was administered to the 12 Subjects from Example 5i. All subjects experienced a greater delay in the onset of bitterness. The subjects described the sweetness as natural and not over powering or artificial.

Example 4k: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 29.61 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Bitter Stop PCCA | 1.4 | ml | No Effect |
| BitterStop Medisca | 1.4 | ml | No Effect |
| Spearmint | 0.35 | ml | No effect |

Patient study results: Troche was administered to Subjects. Subjects reported no change in bitterness compared to Example 4j when both liquid Bitter Stop products were used.

Example 4l: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 31.01 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Bitterness Suppressor Flavor | 1.4 | ml | No Effect |
| Spearmint | 0.35 | ml | No Effect |

Patient study results: Troche was administered to the 18 Subjects. Subjects reported no change in bitterness compared to Examples 4k and 4l when both liquid Bitter Suppressor Flavor products were used.

Example 4m: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 31.41 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Chocolate | 1 | ml | No Effect |
| Spearmint | 0.35 | ml | No Effect |

Patient study results: Troche was administered to Subjects. Subjects reported taste and bitter suppression; however, subjects still reported a bitterness that is felt in the palate and aftertaste of bitterness.

Example 4n: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 29.41 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Butterscotch | 1 | ml | No Effect |
| Caramel | 2 | ml | No Effect |

Patient study results: Troche was administered to 10 Subjects. Subjects reported no significant change compared to Example 4m with the addition of Butterscotch and Caramel to the composition.

Example 4o: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 31.49 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| ButterCream | 1 | ml | No Effect |
| Spearmint | 0.35 | ml | No Effect |

Patient study results: Troche was administered to the 10 Subjects from Example 4n. Subjects reported no significant change compared to Example 4n with the addition of ButterCream to the composition.

Example 4o: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 29.41 | g | 10 mins same |
| Acesulfame | 0.15 | g | No effect |
| Steviol | 0.15 | g | No effect |
| Sildenafil | 1.4 | g | No effect |
| Silica | 0.2 | g | No effect |
| Acacia | 0.2 | g | No effect |
| Grapefruit Extract (NF01) | 1 | gram | No Effect |
| Vanilla | 1 | ml | No Effect |
| Marshmallow | 2 | ml | No Effect |
| Spearmint | 0.35 | ml | No Effect |

Patient study results: Troche was administered to 30 Subject. All subjects reported no bitterness. Thirteen subjects reported that the composition was slightly too sweet. Ten subjects reported that there is a mild aftertaste.

The following table describes the taste reported by the subjects administered the composition of Example 4O:

| # of | | | | Overall | |
|---|---|---|---|---|---|
| Subjects | Taste | Bitterness | Sweetness | Experience | Aftertaste |
| 4 | PLEASANT | NO | Too Much | Good | yes |
| 2 | FRESH | NO | Could be | Delicious | yes |
| | | | less | | |
| 1 | PLEASANT | NO | Okay | Not Great | Metallic |
| 5 | GOOD | NO | Okay | Love it | Somewhat |
| 2 | NICE | NO | Too Much | Fresh and | Artificial |
| | | | | Minty | |
| 10 | PLEASANT | NO | Too much | Okay/Could | None |
| | | | | be better | |
| 6 | FRESH | NO | Too Much | Nice | Somewhat |

Example 4p: Using the process of Example 1, a sublingual formulation was prepared containing the following ingredients:

| | | |
|---|---|---|
| Ingredient | Amount | Dissolving time |

| | | | |
|---|---|---|---|
| PEG1450 | 29.92 | g | 10 mins same |
| Acesulfame | 0.175 | g | No effect |
| Steviol | 0.175 | g | No effect |
| Sildenafil | 1.4 | g | No effect |
| Silica | 0.175 | g | No effect |
| Acacia | 0.175 | g | No effect |
| Grapefruit Extract (NF01) | 1 | gram | No Effect |
| Chocolate | 1 | ml | No Effect |
| Marshmallow | 1 | ml | No Effect |
| Vanilla | 0.2 | ml | |
| Spearmint | 0.4 | ml | No Effect |

Patient study results: Troche was administered to 100 Subjects. Ninety-two subjects reported enjoying the pleasant fresh taste with no aftertaste of bitterness. Three subjects tasted some aftertaste and five subjects felt the composition could be improved with the flavor of Spearmint. Zero individuals reported Bitterness of any kind.

Example 5:

A troche compositions comprising CBD, taste-masking and/or bitterness-masking agents were prepared.

Using the process of Example 1, a sublingual formulation was prepared containing the following ingredients:

| Ingredient | Amount | |
|---|---|---|
| CBD Isolate | 0.35 | g |
| Acesulfame | 0.175 | g |
| Bitterness reducing agent powder (NF01) | 1.0 | g |
| Steviol glycosides 95% (powder) | 0.175 | g |
| Acacia | 0.175 | g |
| Silica | 0.175 | g |
| Menthol | 0.1 | g |
| Marshmallow flavor liquid | 1 | mL |
| Vanilla flavor liquid | 0.2 | mL |
| Spearmint | 0.15 | mL |
| Chocolate flavor liquid | 1 | mL |
| Polyethylene glycol 1450 crystal | 30.93 | g |

Patient study results: Troche of Example 5 was administered to Subjects. Patients reported no bitterness or unpleasant aftertaste in the throat following administration. In contrast, Subjects who were administered similar CBD-containing troches (i.e., those using different combinations of bitterness reducing agent and flavor agents) reported bitterness and an unpleasant aftertaste in the throat following administration.

Example 6: Sildenafil Troche

Troche compositions comprising sildenafil, taste-masking and/or bitterness-masking agents were prepared using the process in Example 1.

These troche compositions were prepared based on the following observations from the earlier examples:

Bitterness Reducing Agent:

Bitterness reducing agent NF01, sold by Medisca, was used for sildenafil formulations. This agent produced unexpected and surprisingly superior results compared to all other bitterness blocking agents, which failed in the research and development phase. NF01 has 95% minimum purity, and the other components are related substances from Grapefruit extract.

The sildenafil formulations were prepared using 1 gram of Bitterness Reducing Agent NF01 in the batch for up to 40 mg sildenafil in the troche. For every 40 mg increase of Sildenafil powder in the troche, the amount of NF01 in the batch was increased by about 0.5 gram.

Chocolate:

Liquid Chocolate Flavor, sold by Medisca, was used for sildenafil formulations. This liquid chocolate flavor produced unexpected and surprisingly superior results compared to all other liquid chocolate flavor agents, which did not produce as successful of a result in the research and development phase.

The sildenafil formulations were prepared using about 1mL liquid chocolate flavor in the process batch for up to 40 mg of sildenafil in the troche. For every 50 mg increase of sildenafil powder in the troche, the liquid chocolate flavor in the batch was increased by about 0.5 mL.

Spearmint:

Spearmint, sold by PCCA, was used for the sildenafil formulations. This spearmint flavor produced unexpected and surprisingly superior results compared to all other spearmint flavor agents, which did not produce as successful of a result in the research and development phase.

The sildenafil formulations used from about 0.2 mL to about 0.6 mL to mask the sildenafil bitterness.

Vanilla:

Vanilla flavor, sold by Flavor RX, was used for the sildenafil formulations. This vanilla flavor produced unexpected and surprisingly superior results compared to all other vanilla flavor agents; all others produced “horrible” taste in development paste.

Marshmallow:

Liquid marshmallow flavor, sold by Medisca, was used for the sildenafil formulations. This marshmallow flavor produced unexpected and surprisingly superior results compared to all other marshmallow flavor; all other marshmallow flavors did not produce as successful of a result in the research and development phase.

The sildenafil formulations were prepared using about 1 mL liquid marshmallow flavor in the batch for up to 40 mg sildenafil powder in the troche. For every 50 mg increase of sildenafil powder in the troche, the liquid marshmallow flavor in the batch is increased by about 0.5 mL. When chocolate flavor is present, the amount of chocolate and marshmallow flavor are increased in 1:1 Ratio

Menthol:

Menthol crystals from PCCA were used.

For formulations containing up to 60 mg sildenafil powder, 0.1 g menthol crystals were used in the batch. For formulations containing from 61 mg to 120 mg sildenafil powder, 0.2 g menthol crystals were used in the batch. For formulations containing from 121 mg to 150 mg sildenafil, 0.3 mg menthol crystals were used in the batch.

Acesulfame and Steviol:

Acesulfame and steviol were added to the batch in about a 1:1 ratio to mask the bitterness. This ratio of acesulfame to steviol is unexpected and surprising because, if the ratio altered, there is an increase in bitterness.

Example 6a.

Using the process of Example 1, a 40 mg sildenafil troche formulation was prepared containing the following ingredients:

| Ingredient | Amount | |
|---|---|---|
| Sildenafil citrate | 1.96 g (equals 1.4 g | |
| | sildenafil) | |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.10 | g |

| | |
|---|---|
| POLYETHYLENE GLYCOL 1450 CRYSTAL | Calculate |
| | Displacement (GM) |
| BLUE (LIQUID) | 1 drop (gtts) |

Example 6b.

Using the process of Example 1, a 65 mg sildenafil troche formulation was prepared containing the following ingredients:

| Ingredient | Amount | |
|---|---|---|
| Sildenafil citrate | 3.19 g (equals 2.28 g | |
| | sildenafil) | |
| BITTERNESS REDUCING AGENT POWDER | 1.2 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMELLOW LIQUID | 1.2 mL | (1.27 mg) |
| VANILLA LIQUID | 0.2 mL | (0.21 mg) |
| CHOCOLATE LIQUID | 1.1 mL | (1.14 mg) |
| ACACIA NF (SPRAY DRIED GUM ARABIC) | 0.175 | g |
| SILICIA GEL MICRONIZED POWDER | 0.175 | g |
| Menthol | 0.12 | g |

| | |
|---|---|
| POLYETHYLENE GLYCOL 1450 CRYSTAL | Calculate |
| | Displacement (GM) |
| BLUE (LIQUID) | 1 drop (gtts) |

Example 6c.

Using the process of Example 1, a 85 mg sildenafil troche formulation was prepared containing the following ingredients:

| Ingredient | Amount | |
|---|---|---|
| Sildenafil citrate | 4.12 g (equals 2.98 g | |
| | sildenafil) | |
| BITTERNESS REDUCING AGENT POWDER | 1.2 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.42 mL | (0.39 mg) |
| MARSHMELLOW LIQUID | 1.2 mL | (1.27 mg) |
| VANILLA LIQUID | 0.2 mL | (0.21) |
| CHOCOLATE LIQUID | 1.2 mL | (1.14 mg) |
| ACACIA NF (SPRAY DRIED GUM ARABIC) | 0.175 | g |
| SILICIA GEL MICRONIZED POWDER | 0.175 | g |
| Menthol | 0.16 | g |

| | |
|---|---|
| POLYETHYLENE GLYCOL 1450 CRYSTAL | Calculate |
| | Displacement (GM) |
| BLUE (LIQUID) | 1 drop (gtts) |

Example 6d.

Using the process of Example 1, a 110 mg sildenafil troche formulation was prepared containing the following ingredients:

| Ingredient | Amount | |
|---|---|---|
| Sildenafil citrate | 5.4 g (equals 3.85 g | |
| | sildenafil) | |
| BITTERNESS REDUCING AGENT POWDER | 1.5 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.45 mL | (0.42 mg) |
| MARSHMELLOW LIQUID | 1.5 mL | (1.59 mg) |
| VANILLA LIQUID | 0.4 mL | (0.47 mg) |
| CHOCOLATE LIQUID | 1.5 mL | (1.56 mg) |
| ACACIA NF (SPRAY DRIED GUM ARABIC) | 0.175 | g |
| SILICIA GEL MICRONIZED POWDER | 0.175 | g |
| Menthol | 0.2 | g |

| | |
|---|---|
| POLYETHYLENE GLYCOL 1450 CRYSTAL | Calculate |
| | Displacement (GM) |
| BLUE (LIQUID) | 1 drop (gtts) |

Example 6e.

Using the process of Example 1, a 150 mg sildenafil troche formulation was prepared containing the following ingredients:

| Ingredient | Amount | |
|---|---|---|
| Sildenafil citrate | 7.4 g (equals 5.25 g | |
| | sildenafil) | |
| BITTERNESS REDUCING AGENT POWDER | 1.5 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.45 mL | (0.42 mg) |
| MARSHMELLOW LIQUID | 1.5 mL | (1.59 mg) |
| VANILLA LIQUID | 0.4 mL | (0.43 mg) |
| CHOCOLATE LIQUID | 1.5 mL | (1.56 mg) |
| ACACIA NF (SPRAY DRIED GUM ARABIC) | 0.175 | g |
| SILICIA GEL MICRONIZED POWDER | 0.175 | g |
| Menthol | 0.2 | g |

| | |
|---|---|
| POLYETHYLENE GLYCOL 1450 CRYSTAL | Calculate |
| | Displacement (GM) |
| BLUE (LIQUID) | 1 drop (gtts) |

Example 7

Troche compositions comprising 12 mg, 22 mg, or 30 mg of tadalafil, taste-masking and/or bitterness-masking agents were prepared using the process in Example 1 and the ingredients in Example 6. Surprisingly, the combination of taste-masking and/or bitterness-masking agents used for the sildenafil troche was effective to mask the bitter taste of 12 mg, 22 mg, or 30 mg of tadalafil.

The tadalafil troche formulations were prepared containing the following ingredients:

| Ingredient | Amount | |
|---|---|---|
| Tadalafil | 0.42 g, 0.77 g, | |
| | or 1.05 mg | |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.10 | g |

| | |
|---|---|
| POLYETHYLENE GLYCOL 1450 CRYSTAL | Calculate |
| | Displacement (GM) |
| BLUE (LIQUID) | 1 drop (gtts) |

Example 8: Sildenafil+Tadalafil Troche

Troche compositions comprising sildenafil and tadalafil, taste-masking and/or bitterness-masking agents were prepared using the process in Example 1 and the ingredients in Example 6. The amount of sildenafil and tadalafil in the troche follows: 40 mg sildenafil/12 mg tadalafil, 65 mg sildenafil/22 mg tidalafil, 85 mg sildenafil/30 mg tadalafil, 110 mg sildenafil/30 mg tadalafil, and 150 mg sildenafil/30 mg tidalafil.

The observations regarding the bitterness reducing agent, chocolate flavor, spearming flavor, vanilla flavor, marshmallow flavor, menthol, and acesulfame:steviol ratio from Example 6 also applied to the sildenafil and tadalafil troche compositions in this Example.

Surprisingly, the sildenafil formulations in Example 6 were also effective to mask the additional bitterness of tadalafil. In other words, the applicant discovered that tadalafil can be added to the formulations in Example 6 and the taste of both sildenafil and tadalafil will be masked without further modifying the amounts of the taste-masking and/or bitterness-masking agents.

Example 8a.

Using the process of Example 1, a 40 mg sildenafil/12 mg tadalafil troche formulation was prepared containing the following ingredients:

| Ingredient | Amount | |
|---|---|---|
| Sildenafil citrate | 1.96 g (equals 1.4 g | |
| | sildenafil) | |
| Tadalafil | 0.42 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.10 | g |

| | |
|---|---|
| POLYETHYLENE GLYCOL 1450 CRYSTAL | Calculate |
| | Displacement (GM) |
| BLUE (LIQUID) | 1 drop (gtts) |

Example 8b.

Using the process of Example 1, a 65 mg sildenafil/22 mg tadalafil troche formulation was prepared containing the following ingredients:

| Ingredient | Amount | |
|---|---|---|
| Sildenafil citrate | 3.19 g (equals 2.28 g | |
| | sildenafil) | |
| Tadalafil | 0.77 | g |
| BITTERNESS REDUCING AGENT POWDER | 1.2 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMELLOW LIQUID | 1.2 mL | (1.27 mg) |
| VANILLA LIQUID | 0.2 mL | (0.21 mg) |
| CHOCOLATE LIQUID | 1.1 mL | (1.14 mg) |
| ACACIA NF (SPRAY DRIED GUM ARABIC) | 0.175 | g |
| SILICIA GEL MICRONIZED POWDER | 0.175 | g |
| Menthol | 0.12 | g |

| | |
|---|---|
| POLYETHYLENE GLYCOL 1450 CRYSTAL | Calculate |
| | Displacement (GM) |
| BLUE (LIQUID) | 1 drop (gtts) |

Example 8c.

Using the process of Example 1, a 85 mg sildenafil/30 mg Tadalafil troche formulation was prepared containing the following ingredients:

| Ingredient | Amount | |
|---|---|---|
| Sildenafil citrate | 4.12 g (equals 2.98 g | |
| | sildenafil) | |
| Tadalafil | 1.05 | g |
| BITTERNESS REDUCING AGENT POWDER | 1.2 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.42 mL | (0.39 mg) |
| MARSHMELLOW LIQUID | 1.2 mL | (1.27 mg) |
| VANILLA LIQUID | 0.2 mL | (0.21) |
| CHOCOLATE LIQUID | 1.2 mL | (1.14 mg) |
| ACACIA NF (SPRAY DRIED GUM ARABIC) | 0.175 | g |
| SILICIA GEL MICRONIZED POWDER | 0.175 | g |
| Menthol | 0.16 | g |

| | |
|---|---|
| POLYETHYLENE GLYCOL 1450 CRYSTAL | Calculate |
| | Displacement (GM) |
| BLUE (LIQUID) | 1 drop (gtts) |

Example 8d.

Using the process of Example 1, a 110 mg sildenafil/30 mg Tadalafil troche formulation was prepared containing the following ingredients:

| Ingredient | Amount | |
|---|---|---|
| Sildenafil citrate | 5.4 g (equals 3.85 g | |
| | sildenafil) | |
| Tadalafil | 1.05 | g |
| BITTERNESS REDUCING AGENT POWDER | 15 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.45 mL | (0.42 mg) |
| MARSHMELLOW LIQUID | 1.5 mL | (1.59 mg) |
| VANILLA LIQUID | 0.4 mL | (0.47 mg) |
| CHOCOLATE LIQUID | 1.5 mL | (1.56 mg) |
| ACACIA NF (SPRAY DRIED GUM ARABIC) | 0.175 | g |
| SILICIA GEL MICRONIZED POWDER | 0.175 | g |
| Menthol | 0.2 | g |

| | |
|---|---|
| POLYETHYLENE GLYCOL 1450 CRYSTAL | Calculate |
| | Displacement (GM) |
| BLUE (LIQUID) | 1 drop (gtts) |

Example 8e.

Using the process of Example 1, a 150 mg sildenafil/30 mg Tadalafil troche formulation was prepared containing the following ingredients:

| Ingredient | Amount | |
|---|---|---|
| Sildenafil citrate | 7.4 g (equals 5.25 g | |
| | sildenafil) | |
| Tadalafil | 1.05 | g |
| BITTERNESS REDUCING AGENT POWDER | 1.5 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.45 mL | (0.42 mg) |
| MARSHMELLOW LIQUID | 1.5 mL | (1.59 mg) |
| VANILLA LIQUID | 0.4 mL | (0.43 mg) |
| CHOCOLATE LIQUID | 1.5 mL | (1.56 mg) |
| ACACIA NF (SPRAY DRIED GUM ARABIC) | 0.175 | g |
| SILICIA GEL MICRONIZED POWDER | 0.175 | g |
| Menthol | 0.2 | g |

| | |
|---|---|
| POLYETHYLENE GLYCOL 1450 CRYSTAL | Calculate |
| | Displacement (GM) |
| BLUE (LIQUID) | 1 drop (gtts) |

Example 9. Sildenafil and Testosterone

Troche compositions comprising sildenafil and testosterone, taste-masking and/or bitterness-masking agents were prepared using the process in Example 1 and the ingredients in Example 6.

The observations regarding the bitterness reducing agent, chocolate flavor, spearming flavor, vanilla flavor, marshmallow flavor, menthol, and acesulfame: steviol ratio from Example 6 also applied to the sildenafil and tadalafil troche compositions in this Example.

Surprisingly, the sildenafil formulations in Example 6 were also effective to mask the taste of testosterone.

Example 9a. 40 mg Sildenafil and 5 mg Testosterone

Using the process of Example 1, a 40 mg sildenafil/5 mg testosterone troche formulation was prepared containing the following ingredients:

| Ingredient | Amount | |
|---|---|---|
| Sildenafil citrate | 1.96 g (equals 1.4 g | |
| | sildenafil) | |
| Testosterone | 0.18 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 9b. 40 mg Sildenafil and 10 mg Testosterone

Using the process of Example 1, a 40 mg sildenafil/10 mg testosterone troche formulation was prepared containing the following ingredients:

| Ingredient | Amount | |
|---|---|---|
| Sildenafil citrate | 1.96 g (equals 1.4 g | |
| | sildenafil) | |
| Testosterone | 0.36 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 10: Tadalafil+Testosterone Troche

Troche compositions comprising tadalafil, testosterone, taste-masking and/or bitterness-masking agents were prepared using the process in Example 1 and the ingredients in Example 6. The amount of tadalafil and testosterone in the troche follows: 14 mg tadalafil/10 mg testosterone, 14 mg tadalafil/12.5 mg testosterone, 14 mg tadalafil/20 mg testosterone, 22 mg tadalafil/30 mg testosterone, and 30 mg tadalafil/30 mg testosterone.

The observations regarding the bitterness reducing agent, chocolate flavor, spearming flavor, vanilla flavor, marshmallow flavor, menthol, and acesulfame:steviol ratio from Example 6 also applied to this Example.

Surprisingly, the tadalafil formulations in Example 7 were also effective to mask the additional bitterness of testosterome. In other words, the applicant discovered that tadalafil can be added to the formulations in Example 7 and the taste of both testosterone and tadalafil will be masked without further modifying the taste-masking and/or bitterness-masking agents.

Example 10a.

Using the process of Example 1, a 14 mg tadalafil/10 mg testosterone troche formulation was prepared containing the following ingredients:

| Ingredient | Amount | |
|---|---|---|
| Tadalafil USP | 0.49 | g |
| Testosterone | 0.42 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 10b.

Using the process of Example 1, a 14 mg tadalafil/12.5 mg testosterone troche formulation was prepared containing the following ingredients:

| Ingredient | Amount | |
|---|---|---|
| Tadalafil USP | 0.49 | g |
| Testosterone | 0.44 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 10c.

Using the process of Example 1, a 14 mg tadalafil/20 mg testosterone troche formulation was prepared containing the following ingredients:

| Ingredient | Amount | |
|---|---|---|
| Tadalafil USP | 0.49 | g |
| Testosterone | 0.70 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 10d.

Using the process of Example 1, a 22 mg tadalafil/30 mg testosterone troche formulation was prepared containing the following ingredients:

| Ingredient | Amount | |
|---|---|---|
| Tadalafil USP | 0.77 | g |
| Testosterone | 1.05 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 10e.

Using the process of Example 1, a 30 mg tadalafil/30 mg testosterone troche formulation was prepared containing the following ingredients:

| Ingredient | Amount | |
|---|---|---|
| Tadalafil USP | 1.05 | g |
| Testosterone | 1.05 | g |
| BITTERNESS REDUCING AGENT POWDER | 0.98 | g |
| (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.9 mL | (0.95 mg) |
| VANILLA LIQUID | 0.2 mL | (0.21 mg) |
| CHOCOLATE LIQUID | 0.9 mL | (0.94 mg) |
| ACACIA NF (SPRAY DRIED GUM ARABIC) | 0.175 | g |
| SILICIA GEL MICRONIZED POWDER | 0.175 | g |
| Menthol | 0.20 | g |

| | |
|---|---|
| POLYETHYLENE GLYCOL 1450 CRYSTAL | Calculate |
| | Displacement (GM) |
| BLUE (LIQUID) | 1 drop (gtts) |

Example 11: Sildenafil+Testosterone+Oxytocin Troche

Troche compositions comprising sildenafil, testosterone, oxytocin, taste-masking and/or bitterness-masking agents were prepared using the process in Example 1 and the ingredients in Example 6. The amount of tadalafil and testosterone in the troche follows: 4 mg sildenafil/2 mg testosterone/20 IU oxytocin, 8 mg sildenafil/4 mg testosterone/20 IU oxytocin, and 10 mg sildenafil/8 mg testosterone/40 IU oxytocin.

The observations regarding the bitterness reducing agent, chocolate flavor, spearming flavor, vanilla flavor, marshmallow flavor, menthol, and acesulfame:steviol ratio from Example 6 also applied to the compositions in this Example.

Surprisingly, the tadalafil formulations in Example 7 were also effective to mask the additional bitterness of testosterone. In other words, the applicant discovered that tadalafil can be added to the formulations in Example 7 and the taste of both testosterone and tadalafil will be masked without further modifying the taste-masking and/or bitterness-masking agents.

Example 11a.

Using the process of Example 1, a 4 mg sildenafil/2 mg testosterone/20 IU oxytocin testosterone troche formulation was prepared containing the following ingredients:

| Ingredient | Amount | |
|---|---|---|
| Sildenafil citrate | 0.20 | g |
| Oxytocin (stock solution = 1 IU/mg) | 0.02 | g |
| Testosterone | 0.07 | g |
| BITTERNESS REDUCING AGENT | 0.98 | g |
| POWDER (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.7 mL | (0.95 mg) |
| VANILLA LIQUID | 0.0875 mL | (0.21 mg) |
| CHOCOLATE LIQUID | 0.0875 mL | (0.94 mg) |
| ACACIA NF (SPRAY DRIED GUM | 0.175 | g |
| ARABIC) | | |
| SILICIA GEL MICRONIZED POWDER | 0.175 | g |

| | |
|---|---|
| POLYETHYLENE GLYCOL 1450 CRYSTAL | Calculate |
| | Displacement (GM) |
| BLUE (LIQUID) | 1 drop (gtts) |

Example 11b.

Using the process of Example 1, a 8 mg sildenafil/4 mg testosterone/20 IU oxytocin testosterone troche formulation was prepared containing the following ingredients:

| Ingredient | Amount | |
|---|---|---|
| Sildenafil citrate | 0.39 | g |
| Oxytocin (stock solution = 1 IU/mg) | 0.02 | g |
| Testosterone | 0.14 | g |
| BITTERNESS REDUCING AGENT | 0.98 | g |
| POWDER (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.7 mL | (0.95 mg) |
| VANILLA LIQUID | 0.0875 mL | (0.21 mg) |
| CHOCOLATE LIQUID | 0.0875 mL | (0.94 mg) |
| ACACIA NF (SPRAY DRIED GUM | 0.175 | g |
| ARABIC) | | |
| SILICIA GEL MICRONIZED POWDER | 0.175 | g |

| | |
|---|---|
| POLYETHYLENE GLYCOL 1450 CRYSTAL | Calculate |
| | Displacement (GM) |
| BLUE (LIQUID) | 1 drop (gtts) |

Example 11c.

Using the process of Example 1, a 10 mg sildenafil/8 mg testosterone/40 IU oxytocin testosterone troche formulation was prepared containing the following ingredients:

| Ingredient | Amount | |
|---|---|---|
| Sildenafil citrate | 0.49 | g |
| Oxytocin (stock solution = 1 IU/mg) | 0.04 | g |
| Testosterone | 0.14 | g |
| BITTERNESS REDUCING AGENT | 0.98 | g |
| POWDER (NF01) | | |
| ACESULFAME POTASSIUM POWDER | 0.175 | g |
| STEVIOL GLYCOSIDES 95% (POWDER) | 0.175 | g |
| SPEARMINT OIL | 0.35 mL | (0.33 mg) |
| MARSHMALLOW LIQUID | 0.7 mL | (0.95 mg) |
| VANILLA LIQUID | 0.0875 mL | (0.21 mg) |
| CHOCOLATE LIQUID | 0.0875 mL | (0.94 mg) |
| ACACIA NF (SPRAY DRIED GUM | 0.175 | g |
| ARABIC) | | |
| SILICIA GEL MICRONIZED POWDER | 0.175 | g |

| | |
|---|---|
| POLYETHYLENE GLYCOL 1450 CRYSTAL | Calculate |
| | Displacement (GM) |
| BLUE (LIQUID) | 1 drop (gtts) |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. A sublingual dosage form comprising one or more of any of the bitter active ingredients disclosed herein, and a bitterness reducing agent.

2. The sublingual dosage form of claim 1, wherein the bitter active ingredient is selected from the group consisting of sildenafil, tadalafil, oxytocin, testosterone, and mixtures thereof.

3. The sublingual dosage form of claim 1, wherein the bitter active ingredient comprises sildenafil.

4. The sublingual dosage form of claim 1, wherein the bitter active ingredient comprises sildenafil citrate.

5. The sublingual dosage form of any one of claims 3-4, wherein the dosage form comprises about 40 mg, about 65 mg, about 80 mg, about 110 mg or about 150 mg of sildenafil or a pharmaceutically acceptable salt thereof.

6. The sublingual dosage form of claim 2, wherein the bitter active ingredient comprises tadalafil.

7. The sublingual dosage form of claim 6, wherein the dosage form comprises about 14 mg, about 22 mg or about 30 mg of tadalafil or a pharmaceutically acceptable salt thereof.

8. The sublingual dosage form of claim 2, wherein the bitter active ingredient comprises testosterone.

9. The sublingual dosage form of claim 8, wherein the dosage form comprises about 2 mg, about 5 mg, about 10 mg, about 12.5 mg, about 20 mg or about 30 mg of testosterone.

10. The sublingual dosage form of claim 2, wherein the bitter active ingredient comprises sildenafil and tadalafil, wherein the tadalafil and sildenafil are optionally and independently a pharmaceutically acceptable salt thereof.

11. The sublingual dosage form of claim 10, wherein the dosage form comprises about 40 mg of sildenafil and about 14 mg of tadalafil; about 65 mg of sildenafil and about 22 mg of tadalafil; about 80 mg of sildenafil and 30 mg of tadalafil; about 110 mg of sildenafil and about 30 mg of tadalafil; about 150 mg of sildenafil and about 30 mg of tadalafil, wherein the tadalafil and sildenafil are optionally and independently a pharmaceutically acceptable salt thereof.

12. The sublingual dosage form of claim 2, wherein the bitter active ingredient comprises tadalafil or a pharmaceutically acceptable salt thereof and testosterone.

13. The sublingual dosage form of claim 12, wherein the dosage form comprises about 14 mg of Tadalafil and about 10 mg of Testosterone, about 14 mg of Tadalafil and about 12.5 mg of Testosterone, about 14 mg of Tadalafil and about 20 mg Testosterone, about 22 mg of Tadalafil and about 30 mg of Testosterone or about 30 mg of Tadalafil and about 30 mg of Testosterone, wherein the Tadafil is optionally a pharmaceutically acceptable salt thereof.

14. The sublingual dosage form of claim 2, wherein the bitter active ingredient comprises sildenafil or a pharmaceutically acceptable salt thereof and testosterone.

15. The sublingual dosage form of claim 14, wherein the dosage form comprises about 35 mg to about 45 mg of Sildenafil and from about 10 mg to about 30 mg of Testosterone, about 40 mg of Sildenafil and about 5 mg Testosterone, about 40 mg of Sildenafil and about 10 mg Testosterone, about 40 mg of Sildenafil and about 12.5 mg Testosterone, about 40 mg of Sildenafil and about 20 mg Testosterone, about 40 mg of Sildenafil and about 30 mg Testosterone, wherein the Sildenafil is optionally a pharmaceutically acceptable salt thereof.

16. The sublingual dosage form of claim 2, wherein the bitter active ingredient comprises sildenafil or a pharmaceutically acceptable salt thereof, tadalafil or a pharmaceutically acceptable salt thereof and testosterone.

17. The sublingual dosage form of claim 16, wherein the dosage form comprises about 40 mg of Sildenafil, about 14 mg of Tadalafil, and about 5 mg of Testosterone; about 40 mg of Sildenafil, about 14 mg of Tadalafil, and about 10 mg of Testosterone; about 40 mg of Sildenafil, about 14 mg of Tadalafil, and about 12.5 mg of Testosterone; about 40 mg of Sildenafil, about 14 mg of Tadalafil, and about 20 mg of Testosterone; about 40 mg of Sildenafil, about 14 mg of Tadalafil, and about 30 mg of Testosterone; about 65 mg of Sildenafil, about 22 mg of Tadalafil, and about 5 mg of Testosterone; about 65 mg of Sildenafil, about 22 mg of Tadalafil, and about 10 mg of Testosterone; about 65 mg of Sildenafil, about 22 mg of Tadalafil, and about 12.5 mg of Testosterone; about 65 mg of Sildenafil, about 22 mg of Tadalafil, and about 20 mg of Testosterone; about 65 mg of Sildenafil, about 22 mg of Tadalafil, and about 30 mg of Testosterone; about 80 mg of Sildenafil, about 30 mg of Tadalafil, and about 30 mg of Testosterone; about 110 mg of Sildenafil, about 30 mg of Tadalafil, and about 30 mg of Testosterone; or about 150 mg of Sildenafil, about 30 mg of Tadalafil, and about 30 mg of Testosterone, wherein the tadalafil and sildenafil are optionally and independently a pharmaceutically salt thereof.

18. The sublingual dosage form of claim 2, wherein the bitter active ingredient comprises oxytocin or a pharmaceutically acceptable salt thereof.

19. The sublingual dosage form of claim 2, wherein the bitter active ingredient comprises oxytocin, sildenafil and testosterone, wherein the oxytocin and sildenafil are optionally and independently a pharmaceutically salt thereof.

20. The sublingual dosage form of claim 19, wherein the dosage form comprises about 4 mg of Sildenafil, about 2 mg of Testosterone and about 20 IU of Oxytocin, about 8 mg of Sildenafil, about 4 mg of Testosterone and about 20 IU of Oxytocin, or about 10 mg of Sildenafil, about 8 mg of Testosterone, and about 40 IU of Oxytocin, wherein the sildenafil is optionally a pharmaceutically salt thereof.

21. The sublingual dosage form of any one of claims 1-20, further comprising one or more of oxytocin, tadalafil, papaverine, or testosterone.

22. The sublingual dosage form of any one of claims 1 to 21, further comprising a sweetener or flavoring agent.

23. The sublingual dosage form of claim 22, wherein the sweetener or flavoring agent is selected from one or more of spearmint oil, marshmallow liquid, vanilla flavor liquid, chocolate flavor liquid, lemon flavoring, or orange flavoring.

24. The sublingual dosage form of any one of claims 1 to 23, further comprising a colorant.

25. The sublingual dosage form of any one of claims 1 to 24, further comprising one or more of acesulfame potassium, steviol, acacia, silica, polyethylene glycol, or a pharmaceutical additive.

26. A sublingual dosage form comprising sildenafil citrate, bitterness reducing agent (NF01), acesulfame potassium, steviol, spearmint oil, marshmallow, vanilla, chocolate, spray dried gum Arabic, polyethylene glycol, and a colorant.

27. The sublingual dosage form of any one of claims 3-5, wherein the composition comprises:
about 2 wt. % to about 6 wt. % of sildenafil or a pharmaceutically acceptable salt thereof,
about 1 wt. % to about 5 wt. % of a bitterness reducing agent; and
about 6 wt. % to about 10 wt. % of a sweetener or flavoring agent.

28. The sublingual dosage form of any one of claims 3-5, wherein the composition comprises:
about 3 wt. % to about 5 wt. % of sildenafil or a pharmaceutically acceptable salt thereof,
about 2 wt. % to about 4 wt. % of a bitterness reducing agent; and
about 7 wt. % to about 9 wt. % of a sweetener or flavoring agent.

29. The sublingual dosage form of any one of claims 3-5, wherein the composition comprises:
about 4 wt. % of sildenafil or a pharmaceutically acceptable salt thereof,
about 3 wt. % of a bitterness reducing agent; and
about 8 wt. % of a sweetener or flavoring agent.

30. The sublingual dosage form of any one of claims 28-29, wherein the bitterness reducing agent comprises NF01 and the sweetener or flavoring agent comprises acesulfame, steviol, silica, acacia, chocolate, marshmallow, vanilla and spearmint.

31. The sublingual dosage form of claim 30, wherein the composition comprises:
about 3 wt. % to about 5 wt. % of sildenafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of silica,
about 0.25 wt % to about 0.75 wt. % of acacia,
about 1 wt % to about 4 wt. % of chocolate,
about 1 wt % to about 4 wt. % of marshmallow,
about 0.4 wt % to about 0.8 wt. % of vanilla and
about 0.5 wt % to about 1.5 wt. % of spearmint.

32. The sublingual dosage form of claim 30, wherein the composition comprises:
about 4 wt. % of sildenafil or a pharmaceutically acceptable salt thereof,
about 3 wt. % NF01,
about 0.5 wt. % acesulfame,
about 0.5 wt. % steviol,
about 0.5 wt. % silica,
about 0.5 wt. % acacia,
about 3 wt. % chocolate,
about 3 wt. % marshmallow,
about 0.6 wt. % vanilla and
about 1 wt. % spearmint.

33. The sublingual dosage form of any one of claims 10-11 wherein the composition comprises:
about 0.2 wt. % to about 1.8 wt. % of sildenafil or a pharmaceutically acceptable salt thereof;
about 0.05 wt. % to about 0.35 wt. % of tadalafil or a pharmaceutically acceptable salt thereof;
about 1 wt. % to about 5 wt. % of a bitterness reducing agent; and
about 4 wt. % to about 8 wt. % of a sweetener or flavoring agent.

34. The sublingual dosage form of any one of claims 10-11, wherein the composition comprises:
about 0.5 wt. % to about 1.5 wt. % of sildenafil or a pharmaceutically acceptable salt thereof;
about 0.1 wt. % to about 0.3 wt. % of tadalafil or a pharmaceutically acceptable salt thereof;
about 2 wt. % to about 4 wt. % of a bitterness reducing agent; and
about 5 wt. % to about 7 wt. % of a sweetener or flavoring agent.

35. The sublingual dosage form of any one of claims 10-11, wherein the composition comprises:
about 1 wt. % of sildenafil or a pharmaceutically acceptable salt thereof,
about 0.2 wt. % of tadalafil or a pharmaceutically acceptable salt thereof
about 3 wt. % of a bitterness reducing agent; and
about 6 wt. % of a sweetener or flavoring agent.

36. The sublingual dosage form of any one of claims 33-35, wherein the bitterness reducing agent comprises NF01 and the sweetener or flavoring agent comprises acesulfame, steviol, spearmint, marshmallow, vanilla, chocolate, and acacia.

37. The sublingual dosage form of claim 36, wherein the composition comprises:
about 0.5 wt. % to about 1.5 wt. % of sildenafil or a pharmaceutically acceptable salt thereof,
about 0.1 wt. % to about 0.3 wt. % of tadalafil or a pharmaceutically acceptable salt thereof,
about 2 wt. % to about 4 wt. % of NF01,
about 0.25 wt % to about 0.75 wt. % of acesulfame,
about 0.25 wt % to about 0.75 wt. % of steviol,
about 0.5 wt % to about 1.5 wt. % of spearmint,
about 1 wt % to about 3 wt. % of marshmallow,
about 0.1 wt % to about 0.5 wt. % of vanilla,
about 0.1 wt % to about 0.5 wt. % of chocolate, and
about 0.25 wt % to about 0.75 wt. % of acacia.

38. The sublingual dosage form of claim 36, wherein the composition comprises:
about 1 wt. % of sildenafil or a pharmaceutically acceptable salt thereof,
about 0.2 wt. % of tadalafil or a pharmaceutically acceptable salt thereof,
about 3 wt. % of NF01,
about 0.5% of acesulfame,
about 0.5% of steviol,
about 1 wt. % of spearmint,
about 2 wt. % of marshmallow,
about 0.3 wt % of vanilla,
about 0.3 wt % of chocolate, and
about 0.5% of acacia.

39. A sublingual dosage form comprising a cannabis extract and a bitterness reducing agent.

40. A sublingual dosage form comprising cannabidiol or a pharmaceutically acceptable salt, ester, prodrug, or solvate thereof and a bitterness reducing agent.

41. The sublingual dosage form of claim 35, wherein the composition comprises:
about 0.2 wt. % to about 1.8 wt. % of cannabidiol or a pharmaceutically acceptable salt, ester, prodrug, or solvate thereof;
about 1 wt. % to about 5 wt. % of a bitterness reducing agent; and
about 6 wt. % to about 10 wt. % of a sweetener or flavoring agent.

42. The sublingual dosage form of claim 35, the composition comprises:
about 0.5 wt. % to about 1.5 wt. % of cannabidiol or a pharmaceutically acceptable salt, ester, prodrug, or solvate thereof, ester, prodrug, or solvate thereof;
about 2 wt. % to about 4 wt. % of a bitterness reducing agent; and
about 7 wt. % to about 9 wt. % of a sweetener or flavoring agent.

43. The sublingual dosage form of claim 35, wherein the composition comprises:
about 1 wt. % of cannabidiol or a pharmaceutically acceptable salt, ester, prodrug, or solvate thereof,
about 3 wt. % of a bitterness reducing agent; and
about 8 wt. % of a sweetener or flavoring agent.

44. A sublingual dosage form comprising tetrahydrocannabinol or a pharmaceutically acceptable salt, ester, prodrug, or solvate thereof (THC) and a bitterness reducing agent.

45. A sublingual dosage form comprising cannabidiol or a pharmaceutically acceptable salt, ester, prodrug, or solvate thereof (CBD) and a bitterness reducing agent.

46. A sublingual dosage form comprising CBD and THC and a bitterness reducing agent.

47. The sublingual dosage form of any one of claims 44-46, wherein the bitterness reducing agent comprises NF01 and the sweetener or flavoring agent comprises acesulfame, steviol, acacia, silica, menthol, chocolate, marshmallow, spearmint and vanilla.

48. The sublingual dosage form of claim 47, wherein the composition comprises:
about 0.5 wt. % to about 1.5 wt. % of cannabidiol or a pharmaceutically acceptable salt, ester, prodrug, or solvate thereof,
about 2 wt. % to about 4 wt. % of NF01,
about 0.25 wt % to about 0.75 wt. % of acesulfame,
about 0.25 wt % to about 0.75 wt. % of steviol,
about 0.25 wt % to about 0.75 wt. % of acacia,
about 0.25 wt % to about 0.75 wt. % of silica,
about 0.1 wt % to about 0.4 wt. % of menthol,
about 2 wt % to about 4 wt. % of chocolate,
about 2 wt % to about 4 wt. % of marshmallow,
about 0.3 wt % to about 0.5 wt. % of spearmint, and
about 0.5 wt % to about 0.7 wt. % of vanilla.

49. The sublingual dosage form of claim 47, wherein the composition comprises:
about 1 wt. % of cannabidiol or a pharmaceutically acceptable salt, ester, prodrug, or solvate thereof,
about 3 wt. % of NF01,
about 0.5 wt. % of acesulfame,
about 0.5 wt. % of steviol,
about 0.5 wt. % of acacia,
about 0.5 wt. % of silica,
about 0.3 wt. % of menthol,
about 3 wt. % of chocolate,
about 3 wt. % of marshmallow,
about 0.4 wt % of spearmint, and
about 0.6 wt % of vanilla.

50. The sublingual dosage form of any one of claim 1-49, wherein the dosage form is a troche.

51. The sublingual dosage form of any one of claims 1-50, wherein the bitterness reducing agent comprises NF01.

52. The sublingual dosage form of claim 51, wherein the NF01 exhibits a UV/visible spectrum comprising an absorption band at about 288 nm.

53. The sublingual dosage form of claim 51, wherein the NF01 exhibits a UV/visible spectrum comprising a maximum absorption band at about 288 nm.

54. The sublingual dosage form of claim 51, wherein the NF01 exhibits a UV/visible spectrum that is substantially similar to FIG. 1 (solid line).

55. The sublingual dosage form of any one of claims 1-54, wherein the sweetener or flavoring agent comprises acacia.

56. The sublingual dosage form of claim 55, wherein the acacia exhibits a UV/visible spectrum comprising an absorption band at about 277 nm.

57. The sublingual dosage form of claim 55, wherein the acacia exhibits a UV/visible spectrum comprising a maximum absorption band at about 277 nm. I

58. The sublingual dosage form of claim 55, wherein the acacia exhibits a UV/visible spectrum that is substantially similar to FIG. 2 (broken line).

59. The sublingual dosage form of any one of claims 1-58, wherein the sweetener or flavoring agent comprises steviol.

60. The sublingual dosage form of claim 59, wherein the steviol exhibits a UV/visible spectrum comprising an absorption band at about 252 nm.

61. The sublingual dosage form of claim 60, wherein the steviol exhibits a UV/visible spectrum comprising a maximum absorption band at about 252 nm.

62. The sublingual dosage form of claim 60, wherein the steviol exhibits a UV/visible spectrum that is substantially similar to FIG. 3 (broken line).

63. The sublingual dosage form of any one of claims 1-62, wherein sweetener or flavoring agent comprise spearmint oil.

64. The sublingual dosage form of claim 63, wherein spearmint oil exhibits a UV/visible spectrum comprising an absorption band at about 252 nm.

65. The sublingual dosage form of claim 64, wherein the spearmint oil exhibits a UV/visible spectrum comprising a maximum absorption band at about 252 nm.

66. The sublingual dosage form of claim 64, wherein the spearmint oil exhibits a UV/visible spectrum that is substantially similar to FIG. 4 (broken line).

67. The sublingual dosage form of any one of claims 1-66, wherein the sweetener or flavoring agent comprises acesulfame potassium.

68. The sublingual dosage form of claim 67, wherein the acesulfame potassium exhibits a UV/visible spectrum comprising an absorption band at about 252 nm.

69. The sublingual dosage form of claim 67, wherein the acesulfame potassium exhibits a UV/visible spectrum comprising a maximum absorption band at about 252 nm.

70. The sublingual dosage form of claim 67, wherein the acesulfame potassium exhibits a UV/visible spectrum that is substantially similar to FIG. 5 (broken line).

71. The sublingual dosage form of any one of claims 1-70, wherein the sweetener or flavoring agent comprises menthol.

72. The sublingual dosage form of claim 71, wherein the menthol exhibits a UV/visible spectrum comprising an absorption band at about 283 nm.

73. The sublingual dosage form of claim 72, wherein the menthol exhibits a UV/visible spectrum comprising a maximum absorption band at about 283 nm.

74. The sublingual dosage form of claim 72, wherein the menthol exhibits a UV/visible spectrum that is substantially similar to FIG. 6 (broken line).

75. The sublingual dosage form of any one of claims 1-74, wherein the sweetener or flavoring agent comprises silica.

76. The sublingual dosage form of claim 75, wherein the silica exhibits a UV/visible spectrum comprising an absorption band at about 321 nm.

77. The sublingual dosage form of claim 75, wherein the silica exhibits a UV/visible spectrum comprising a maximum absorption band at about 321 nm.

78. The sublingual dosage form of claim 75, wherein the silica exhibits a UV/visible spectrum that is substantially similar to FIG. 7 (broken line).

79. The sublingual dosage form of any one of claims 1-78, wherein the sweetener or flavoring agent comprises marshmallow.

80. The sublingual dosage form of claim 79, wherein the marshmallow exhibits a UV/visible spectrum comprising an absorption band at about 277 nm.

81. The sublingual dosage form of claim 79, wherein the marshmallow exhibits a UV/visible spectrum comprising a maximum absorption band at about 277 nm.

82. The sublingual dosage form of claim 79, wherein the marshmallow exhibits a UV/visible spectrum that is substantially similar to FIG. 8 (broken line).

83. The sublingual dosage form of any one of claims 1-82, wherein the sweetener or flavoring agent comprises vanilla.

84. The sublingual dosage form of claim 77, wherein the vanilla exhibits a UV/visible spectrum comprising absorption bands at about 277 nm and about 308 nm.

85. The sublingual dosage form of claim 84, wherein the vanilla exhibits a UV/visible spectrum comprising a maximum absorption band at about 277 nm.

86. The sublingual dosage form of claim 84, wherein the vanilla exhibits a UV/visible spectrum that is substantially similar to FIG. 9 (broken line).

87. The sublingual dosage form of any one of claims 1-86, wherein the sweetener or flavoring agent comprises chocolate.

88. The sublingual dosage form of claim 87, wherein the chocolate comprises vanillin and exhibits a UV/visible spectrum comprising an absorption band at about 277 nm.

89. The sublingual dosage form of claim 87, wherein the chocolate comprises vanillin, butyraldehyde and phenol and exhibits a UV/visible spectrum comprising an absorption band at about 277 nm.

90. The sublingual dosage form of claim 87, wherein the chocolate comprises vanillin and exhibits a UV/visible spectrum that is substantially similar to FIG. 10.

91. The sublingual dosage form of claim 87, wherein the chocolate comprises vanillin, butyraldehyde and phenol and exhibits a UV/visible spectrum that is substantially similar to FIG. 10 (broken line).

92. The sublingual dosage form of any one of claims 1-91, wherein:
the bitterness reducing agent comprises:
NF01, wherein the NF01 exhibits a UV/visible spectrum that is substantially similar to FIG. 1 (solid line); and
the sweetener or flavoring agent comprises:
acacia, wherein the acacia exhibits a UV/visible spectrum that is substantially similar to FIG. 2 (broken line);
steviol, wherein the steviol exhibits a UV/visible spectrum that is substantially similar to FIG. 3 (broken line);
spearmint oil, wherein the spearmint oil exhibits a UV/visible spectrum that is substantially similar to FIG. 4 (broken line);
acesulfame potassium, wherein the acesulfame potassium exhibits a UV/visible spectrum that is substantially similar to FIG. 5 (broken line);
menthol, wherein the menthol exhibits a UV/visible spectrum that is substantially similar to FIG. 6 (broken line);
silica, wherein the silica exhibits a UV/visible spectrum that is substantially similar to FIG. 7 (broken line);
marshmallow, wherein the marshmallow exhibits a UV/visible spectrum that is substantially similar to FIG. 8 (broken line);
vanilla, wherein the vanilla exhibits a UV/visible spectrum that is substantially similar to FIG. 9 (broken line); and
chocolate, wherein the chocolate comprises vanillin and exhibits a UV/visible spectrum that is substantially similar to FIG. 10 (broken line).

93. The sublingual dosage form of any one of claims 1-92, wherein:
the bitterness reducing agent comprises:
NF01, wherein the NF01 exhibits a UV/visible spectrum comprising an absorption band at about 288 nm; and
the sweetener or flavoring agent comprises:
acacia, wherein the acacia exhibits a UV/visible spectrum comprising an absorption band at about 277 nm;
steviol, wherein the steviol exhibits a UV/visible spectrum comprising an absorption band at about 252 nm;
spearmint oil, wherein the spearmint oil exhibits a UV/visible spectrum comprising an absorption band at about 252 nm;
acesulfame potassium, wherein the acesulfame potassium exhibits a UV/visible spectrum comprising an absorption band at about 252 nm;
menthol, wherein the menthol exhibits a UV/visible spectrum comprising an absorption band at about 283 nm;
silica, wherein the silica exhibits a UV/visible spectrum comprising an absorption band at about 321 nm;
marshmallow, wherein the marshmallow exhibits a UV/visible spectrum comprising an absorption band at about 277 nm;
vanilla, wherein the vanilla exhibits a UV/visible spectrum comprising absorption bands at about 277 nm and about 308; and
chocolate, wherein the chocolate exhibits a UV/visible spectrum comprising an absorption band at about 277 nm.

94. A method of treating sexual dysfunction in a male or female patient, the method comprising administering a therapeutically effective amount of the sublingual dosage form of any one of claims 1-93 to a patient in need thereof, wherein the bitter active ingredient comprises sildenafil, tadalafil, oxytocin, or testosterone and mixtures thereof

95. The method of claim 94, wherein the administration comprises placing the dosage form under the tongue of the patient in need thereof for about 1 to about 10 minutes.

96. A method of treating a disorder selected from inflammation and pain comprising administering a therapeutically effective amount of the composition of any one of claims 44-93.

97. The method of claim 96, wherein the pain disorder is selected from the group consisting of acute, chronic, neuropathic or migraine headache pain.

98. A method of treating a disorder selected from insomnia, post-traumatic stress disorder, and anxiety comprising administering a therapeutically effective amount of the composition of any one of claims 44-93.

99. A method of treating a disorder selected from Parkinson's disease, Alzheimer's dementia, Autism Spectrum Disorder and seizures comprising administering a therapeutically effective amount of the composition of any one of claims 44-93.

100. The method of any of claims 96-99, wherein the administration comprises placing the dosage form under the tongue of the patient in need thereof for about 1 to about 10 minutes.

101. The sublingual dosage form of any of claims 1-93, comprising a troche base.

102. The sublingual dosage form of claim 101, wherein the troche base is present in an amount that causes the sublingual dosage form to dissolve within about 6 minute (+/−1 minute) after oral administration.

103. The sublingual dosage form of claim 101, wherein the troche base is present in an amount that causes the sublingual dosage form to dissolve within about 6 minute (+/−1 minute) when tested in the USP <701> Disintegration Test.
